(19) United States
(12) Patent Application Publication
(10) Pub. No.: US 2024/0140951 A1
(43) Pub. Date: May 2, 2024

(54) IMIDAZOPYRIDAZINE DERIVATIVES AS IL-17 MODULATORS

(71) Applicant: UCB Biopharma SRL, Brussels (BE)

(72) Inventors: Gareth Neil Brace, Abingdon, Oxfordshire (GB); Shuyu Chu, Slough, Berkshire (GB); Anne Marie Foley, Slough, Berkshire (GB); James Andrew Johnson, c/o IPD, UCB (GB); Timothy John Norman, Slough, Berkshire (GB); Joanna Rachel Quincey, Slough, Berkshire (GB); James Thomas Reuberson, Slough, Berkshire (GB); Robert Straker, Slough, Berkshire (GB); Robert James Townsend, Abingdon, Oxfordshire (GB)

(21) Appl. No.: 18/267,235

(22) PCT Filed: Dec. 6, 2021
(86) PCT No.: PCT/EP2021/084448

§ 371 Date: Jun. 14, 2023

(30) Foreign Application Priority Data
Dec. 14, 2020 (GB) GB 2019702.6
Jul. 2, 2021 (GB) GB 2109583.1

(51) Int. Cl. C07D 487/04; A61P 29/00; C07D 519/00

(52) U.S. Cl. CPC C07D 487/04; A61P 29/00; C07D 519/00

(57) ABSTRACT

A series of substituted imidazo[1,2-b]pyridazine derivatives as defined herein, being potent modulators of human IL-17 activity, are accordingly of benefit in the treatment and/or prevention of various human ailments, including inflammatory and autoimmune disorders.

The present invention relates to heterocyclic compounds, and to their use in therapy. More particularly, this invention is concerned with pharmacologically active substituted imidazo[1,2-b]pyridazine derivatives. These compounds act as modulators of IL-17 activity, and are accordingly of benefit as pharmaceutical agents for the treatment and/or prevention of pathological conditions, including adverse inflammatory and autoimmune disorders.

IL-17A (originally named CTLA-8 and also known as IL-17) is a pro-inflammatory cytokine and the founder member of the IL-17 family (Rouvier et al., J. Immunol., 1993, 150, 5445-5456). Subsequently, five additional members of the family (IL-17B to IL-17F) have been identified, including the most closely related, IL-17F (ML-1), which shares approximately 55% amino acid sequence homology with IL-17A (Moseley et al., Cytokine Growth Factor Rev., 2003, 14, 155-174). IL-17A and IL-17F are expressed by the recently defined autoimmune related subset of T helper cells, Th17, that also express IL-21 and IL-22 signature cytokines (Korn et al., Ann. Rev. Immunol., 2009, 27, 485-517). IL-17A and IL-17F are expressed as homodimers, but may also be expressed as the IL-17A/F heterodimer (Wright et al., J. Immunol., 2008, 181, 2799-2805). IL-17A and F signal through the receptors IL-17R, IL-17RC or an IL-17RA/RC receptor complex (Gaffen, Cytokine, 2008, 43, 402-407). Both IL-17A and IL-17F have been associated with a number of autoimmune diseases.

The compounds in accordance with the present invention, being potent modulators of human IL-17 activity, are therefore beneficial in the treatment and/or prevention of various human ailments, including inflammatory and autoimmune disorders.

Furthermore, the compounds in accordance with the present invention may be beneficial as pharmacological standards for use in the development of new biological tests and in the search for new pharmacological agents. Thus, the compounds of this invention may be useful as radioligands in assays for detecting pharmacologically active compounds.

WO 2013/116682 and WO 2014/066726 relate to separate classes of chemical compounds that are stated to modulate the activity of IL-17 and to be useful in the treatment of medical conditions, including inflammatory diseases.

WO 2018/229079 and WO 2020/011731 describe spirocyclic molecules that are stated to act as modulators of IL-17 activity, and thus to be of benefit in the treatment of pathological conditions including adverse inflammatory and autoimmune disorders.

WO 2019/138017 describes a class of fused bicyclic imidazole derivatives, including benzimidazole derivatives and analogues thereof, that are stated to act as modulators of IL-17 activity, and thus to be of benefit in the treatment of pathological conditions including adverse inflammatory and autoimmune disorders.

WO 2019/223718 describes heterocyclic compounds, including benzimidazole derivatives, that are stated to inhibit IL-17A and to be useful as immunomodulators.

Heterocyclic compounds stated to be capable of modulating IL-17 activity are also described in WO 2020/127685, WO 2020/146194 and WO 2020/182666.

WO 2020/120140 and WO 2020/120141, and co-pending international patent applications PCT/IB2020/055970, PCT/EP2020/067758 and PCT/EP2020/067759 (all published on 30 Dec. 2020, as WO 2020/261141, WO 2020/260425 and WO 2020/260426 respectively), describe discrete classes of chemical compounds that are stated to act as modulators of IL-17 activity, and thus to be of benefit in the treatment of pathological conditions including adverse inflammatory and autoimmune disorders.

None of the prior art available to date, however, discloses or suggests the precise structural class of substituted imidazo[1,2-b]pyridazine derivatives as provided by the present invention.

As well as being potent modulators of human IL-17 activity, the compounds in accordance with the present invention possess other notable advantages. In particular, the compounds of the invention display valuable metabolic stability, as determined in either microsomal or hepatocyte incubations. The compounds of the invention also display valuable permeability as determined by standard assays, e.g. the Caco-2 permeability assay.

The present invention provides a compound of formula (I) or an N-oxide thereof, or a pharmaceutically acceptable salt thereof:

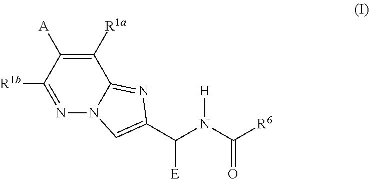

wherein
E represents a group of formula (Ea), (Eb), (Ec), (Ed) or (Ee):

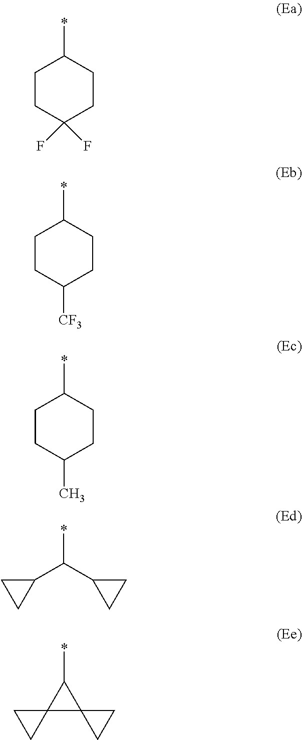

in which the asterisk (*) represents the point of attachment to the remainder of the molecule;
A represents a group of formula (Aa), (Ab), (Ac), (Ad) or (Ae):

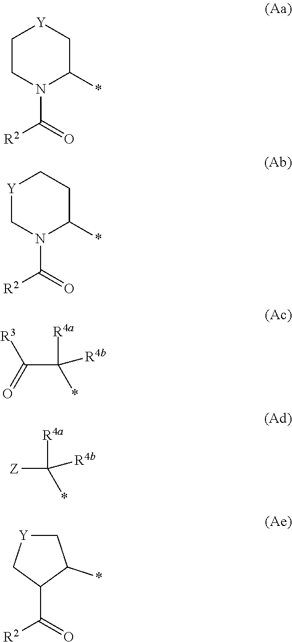

in which the asterisk (*) represents the point of attachment to the remainder of the molecule;
Y represents —O—, —N(R⁷)—, —C(R^{5a})(R^{5b})—, —S—, —S(O)—, —S(O)₂— or —S(O)(N—R⁸)—
Z represents heteroaryl, which group may be optionally substituted by one or more substituents;
R^{1a} represents hydrogen, fluoro, chloro, methyl, difluoromethyl or trifluoromethyl;
R^{1b} represents hydrogen, fluoro, chloro, methyl, difluoromethyl or trifluoromethyl;
R² represents —OR^{2a}; or R² represents C₃₋₉ cycloalkyl, C₄₋₁₂ bicycloalkyl, C₃₋₇ heterocycloalkyl or C₄₋₉ heterobicycloalkyl, any of which groups may be optionally substituted by one or more substituents;
R^{2a} represents C₁₋₆ alkyl; or R^{2a} represents C₃₋₉ cycloalkyl, which group may be optionally substituted by one or more substituents;
R³ represents —NR^{3a}R^{3b}; or R³ represents a group of formula (Wa):

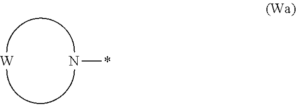

in which the asterisk (*) represents the point of attachment to the remainder of the molecule;
W represents the residue of an optionally substituted saturated monocyclic ring containing 3 to 6 carbon atoms, one nitrogen atom, and 0, 1, 2 or 3 additional heteroatoms independently selected from N, O and S, but containing no more than one O or S atom; or
W represents the residue of an optionally substituted saturated bicyclic ring system containing 4 to 10 carbon atoms, one nitrogen atom, and 0, 1, 2 or 3 additional heteroatoms independently selected from N, O and S, but containing no more than one O or S atom; or
W represents the residue of an optionally substituted saturated spirocyclic ring system containing 5 to 10 carbon atoms, one nitrogen atom, and 0, 1, 2 or 3 additional heteroatoms independently selected from N, O and S, but containing no more than one O or S atom;
R^{3a} represents hydrogen or C₁₋₆ alkyl;
R^{3b} represents C₁₋₆ alkyl, C₃₋₇ cycloalkyl, C₃₋₇ cycloalkyl(C₁₋₆)alkyl, C₄₋₁₂ bicycloalkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents;
R^{4a} represents hydrogen, fluoro or hydroxy; or R^{4a} represents C₁₋₆ alkyl, which group may be optionally substituted by one or more substituents; and
R^{4b} represents hydrogen or fluoro; or R^{4b} represents C₁₋₆ alkyl, which group may be optionally substituted by one or more substituents; or
R^{4a} and R^{4b}, when taken together with the carbon atom to which they are both attached, represent C₃₋₉ cycloalkyl or C₃₋₇ heterocycloalkyl, either of which groups may be optionally substituted by one or more substituents;
R^{5a} represents hydrogen, fluoro, methyl, difluoromethyl or trifluoromethyl; and
R^{5b} represents hydrogen, fluoro, methyl or hydroxy; or
R^{5a} and R^{5b}, when taken together with the carbon atom to which they are both attached, represent cyclopropyl;
R⁶ represents —OR^{6a} or —NR^{6b}R^{6c}; or R⁶ represents C₁₋₆ alkyl, C₃₋₉ cycloalkyl, C₃₋₉ cycloalkyl(C₁₋₆)alkyl, aryl, aryl(C₁₋₆)alkyl, C₃₋₇ heterocycloalkyl, C₃₋₇ heterocycloalkyl-(C₁₋₆)alkyl, heteroaryl or heteroaryl(C₁₋₆)alkyl, any of which groups may be optionally substituted by one or more substituents;
R^{6a} represents C₁₋₆ alkyl; or R^{6a} represents C₃₋₉ cycloalkyl or C₃₋₇ heterocycloalkyl, either of which groups may be optionally substituted by one or more substituents;
R^{6b} represents hydrogen or C₁₋₆ alkyl; and
R^{6c} represents hydrogen or C₁₋₆ alkyl; or
R^{6b} and R^{6c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents;
R⁷ represents —COR^{7a}, —CO₂R^{7a} or —SO₂R^{7b}; or R⁷ represents hydrogen; or R⁷ represents C₁₋₆ alkyl, C₃₋₉ cycloalkyl, or C₃₋₇ heterocycloalkyl, any of which groups may be optionally substituted by one or more fluorine atoms;
R^{7a} represents C₁₋₆ alkyl, optionally substituted by one or more fluorine atoms;
R^{7b} represents C₁₋₆ alkyl; and
R⁸ represents C₁₋₆ alkyl.

The present invention also provides a compound of formula (I) as defined above, or a pharmaceutically acceptable salt thereof.

The present invention also provides a compound of formula (I) as defined above or an N-oxide thereof, or a pharmaceutically acceptable salt thereof, for use in therapy.

The present invention also provides a compound of formula (I) as defined above or an N-oxide thereof, or a pharmaceutically acceptable salt thereof, for use in the treatment and/or prevention of disorders for which the administration of a modulator of IL-17 function is indicated.

The present invention also provides the use of a compound of formula (I) as defined above or an N-oxide thereof, or a pharmaceutically acceptable salt thereof, for the manufacture of a medicament for the treatment and/or prevention of disorders for which the administration of a modulator of IL-17 function is indicated.

The present invention also provides a method for the treatment and/or prevention of disorders for which the administration of a modulator of IL-17 function is indicated which comprises administering to a patient in need of such treatment an effective amount of a compound of formula (I) as defined above or an N-oxide thereof, or a pharmaceutically acceptable salt thereof.

Where any of the groups in the compounds of formula (I) above is stated to be optionally substituted, this group may be unsubstituted, or substituted by one or more substituents. Generally, such groups will be unsubstituted, or substituted by one, two, three or four substituents. Typically, such groups will be unsubstituted, or substituted by one, two or three substituents. Suitably, such groups will be unsubstituted, or substituted by one or two substituents.

For use in medicine, the salts of the compounds of formula (I) will be pharmaceutically acceptable salts. Other salts may, however, be useful in the preparation of the compounds of formula (I) or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. Suitable pharmaceutically acceptable salts of the compounds of formula (I) include acid addition salts which may, for example, be formed by mixing a solution of a compound of formula (I) with a solution of a pharmaceutically acceptable acid.

The present invention also includes within its scope co-crystals of the compounds of formula (I) above. The technical term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity (see Pharmaceutical Salts and Co-crystals, ed. J. Wouters & L. Quere, RSC Publishing, 2012).

Suitable alkyl groups which may be present on the compounds of use in the invention include straight-chained and branched C₁₋₆ alkyl groups, for example C₁₋₄ alkyl groups. Typical examples include methyl and ethyl groups, and straight-chained or branched propyl, butyl and pentyl groups. Particular alkyl groups include methyl, ethyl, n-propyl, isopropyl, n-butyl, sec-butyl, isobutyl, tert-butyl, 2,2-dimethylpropyl and 3-methylbutyl. Derived expressions such as “C₁₋₆ alkoxy”, “C₁₋₆ alkylthio”, “C₁₋₆ alkylsulphonyl” and “C₁₋₆ alkylamino” are to be construed accordingly.

The term “C₃₋₉ cycloalkyl” as used herein refers to monovalent groups of 3 to 9 carbon atoms derived from a saturated monocyclic hydrocarbon, and may comprise benzo-fused analogues thereof. Suitable C₃₋₉ cycloalkyl groups include cyclopropyl, cyclobutyl, benzocyclobutenyl, cyclopentyl, indanyl, cyclohexyl, cycloheptyl, cyclooctyl and cyclononanyl.

The term “C₄₋₁₂ bicycloalkyl” as used herein refers to monovalent groups of 4 to 12 carbon atoms derived from a saturated bicyclic hydrocarbon. Typical bicycloalkyl groups include bicyclo[1.1.1]pentanyl, bicyclo[3.1.0]hexanyl, bicyclo[4.1.0]heptanyl and bicyclo[2.2.2]octanyl.

The term “aryl” as used herein refers to monovalent carbocyclic aromatic groups derived from a single aromatic ring or multiple condensed aromatic rings. Suitable aryl groups include phenyl and naphthyl, preferably phenyl.

Suitable aryl(C₁₋₆)alkyl groups include benzyl, phenylethyl, phenylpropyl and naphthylmethyl.

The term “C₃₋₇ heterocycloalkyl” as used herein refers to saturated monocyclic rings containing 3 to 7 carbon atoms and at least one heteroatom selected from oxygen, sulphur and nitrogen, and may comprise benzo-fused analogues thereof. Suitable heterocycloalkyl groups include oxetanyl, azetidinyl, tetrahydrofuranyl, dihydrobenzo-furanyl, dihydrobenzothienyl, pyrrolidinyl, indolinyl, isoindolinyl, oxazolidinyl, thiazolidinyl, isothiazolidinyl, imidazolidinyl, tetrahydropyranyl, chromanyl, tetrahydro-thiopyranyl, piperidinyl, 1,2,3,4-tetrahydroquinolinyl, 1,2,3,4-tetrahydroisoquinolinyl, piperazinyl, 1,2,3,4-tetrahydroquinoxalinyl, hexahydro-[1,2,5]thiadiazolo[2,3-a]-pyrazinyl, homopiperazinyl, morpholinyl, benzoxazinyl, thiomorpholinyl, azepanyl, oxazepanyl, diazepanyl, thiadiazepanyl and azocanyl.

The term “C₄₋₉ heterobicycloalkyl” as used herein corresponds to C₄₋₉ bicycloalkyl wherein one or more of the carbon atoms have been replaced by one or more heteroatoms selected from oxygen, sulphur and nitrogen. Typical heterobicycloalkyl groups include 6-oxabicyclo[3.1.0]hexanyl, 3-azabicyclo[3.1.0]hexanyl, 2-oxa-5-azabicyclo[2.2.1]-heptanyl, 6-azabicyclo[3.2.0]heptanyl, 6-oxabicyclo[3.1.1]heptanyl, 3-azabicyclo[3.1.1]-heptanyl, 3-azabicyclo[4.1.0]heptanyl, 2-oxabicyclo[2.2.2]octanyl, quinuclidinyl, 2-oxa-5-azabicyclo[2.2.2]octanyl, 8-oxabicyclo[3.2.1]octanyl, 3-azabicyclo[3.2.1]octanyl, 8-azabicyclo[3.2.1]octanyl, 3-oxa-8-azabicyclo[3.2.1]octanyl, 3,8-diazabicyclo[3.2.1]-octanyl, 3,6-diazabicyclo[3.2.2]nonanyl, 3-oxa-7-azabicyclo[3.3.1]nonanyl, 3,7-dioxa-9-azabicyclo[3.3.1]nonanyl and 3,9-diazabicyclo[4.2.1]nonanyl.

The term “heteroaryl” as used herein refers to monovalent aromatic groups containing at least 5 atoms derived from a single ring or multiple condensed rings, wherein one or more carbon atoms have been replaced by one or more heteroatoms selected from oxygen, sulphur and nitrogen. Suitable heteroaryl groups include furyl, benzofuryl, dibenzofuryl, thienyl, benzothienyl, thieno[2,3-c]pyrazolyl, thieno[3,4-b]-[1,4]dioxinyl, dibenzothienyl, pyrrolyl, indolyl, pyrrolo[2,3-b]pyridinyl, pyrrolo[3,2-c]-pyridinyl, pyrrolo[3,4-b]pyridinyl, pyrazolyl, pyrazolo[1,5-a]pyridinyl, 4,5,6,7-tetrahydropyrazolo[1,5-a]pyridinyl, pyrazolo[3,4-d]pyrimidinyl, pyrazolo[1,5-a]-pyrazinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo[2,1-b]-thiazolyl, imidazo[1,2-a]pyridinyl, 5,6,7,8-tetrahydroimidazo[1,2-a]pyridinyl, imidazo-[4,5-b]pyridinyl, imidazo[1,2-b]pyridazinyl, purinyl, imidazo[1,2-a]pyrimidinyl, imidazo-[1,2-c]pyrimidinyl, imidazo[1,2-a]pyrazinyl, oxadiazolyl, thiadiazolyl, triazolyl, [1,2,4]-triazolo[1,5-a]pyridinyl, [1,2,4]triazolo[4,3-a]pyridinyl, [1,2,4]triazolo[4,3-a]pyrazinyl, 5,6,7,8-tetrahydro[1,2,4]triazolo[4,3-a]pyridinyl, [1,2,4]triazolo[1,5-a]pyrimidinyl, 6,8-dihydro-5H-[1,2,4]triazolo[4,3-a]pyrazinyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl and chromenyl groups.

The term “halogen” as used herein is intended to include fluorine, chlorine, bromine and iodine atoms, typically fluorine, chlorine or bromine.

Where the compounds of formula (I) have one or more asymmetric centres, they may accordingly exist as enantiomers. Where the compounds in accordance with the invention possess two or more asymmetric centres, they may additionally exist as diastereomers. The invention is to be understood to extend to the use of all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C═O)↔enol (CH═CHOH) tautomers or amide (NHC═O)↔hydroxyimine (N═COH) tautomers. Formula (I) and the formulae depicted hereinafter are intended to represent all individual tautomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium) or ³H (tritium) atom, preferably ¹H. Similarly, by way of example, each individual carbon atom present in formula (I), or in the formulae depicted hereinafter, may be present as a ¹²C, ¹³C or ¹⁴C atom, preferably ¹²C.

In a first embodiment, E represents a group of formula (Ea). In a second embodiment, E represents a group of formula (Eb). In a third embodiment, E represents a group of formula (Ec). In a fourth embodiment, E represents a group of formula (Ed). In a fifth embodiment, E represents a group of formula (Ee).

Suitably, E represents a group of formula (Ea) or (Ed).

Generally, the present invention provides a compound of formula (IA-1), (IA-2), (IA-3), (IA-4) or (IA-5) or an N-oxide thereof, or a pharmaceutically acceptable salt thereof:

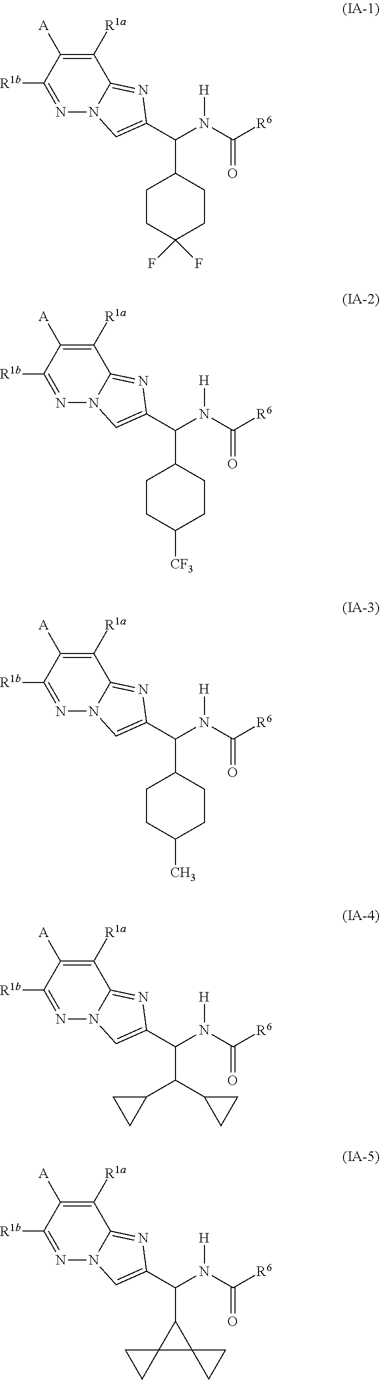

wherein A, R^{1a}, R^{1b} and R⁶ are as defined above.

Suitably, the present invention provides a compound of formula (IA-1) or (IA-4) as defined above or an N-oxide thereof, or a pharmaceutically acceptable salt thereof.

Generally, A represents a group of formula (Aa), (Ab), (Ac) or (Ad).

In a first embodiment, A represents a group of formula (Aa). In a second embodiment, A represents a group of formula (Ab). In a third embodiment, A represents a group of formula (Ac). In a fourth embodiment, A represents a group of formula (Ad).

In a fifth embodiment, A represents a group of formula (Ae).

Typically, A represents a group of formula (Aa), (Ac), (Ad) or (Ae).

Suitably, A represents a group of formula (Ac).

Generally, the present invention provides a compound of formula (IB-1), (IB-2), (IB-3), (IB-4) or (IB-5) or an N-oxide thereof, or a pharmaceutically acceptable salt thereof:

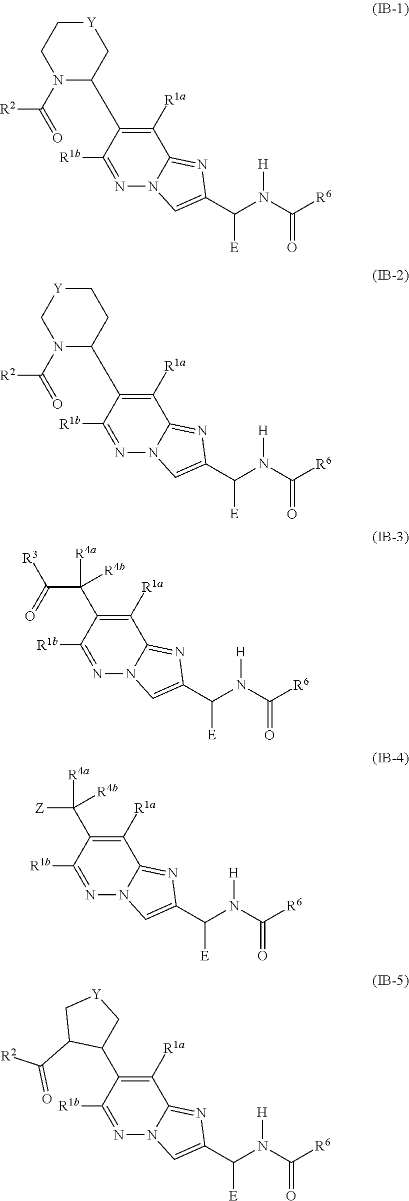

wherein E, Y, Z, R^{1a}, R^{1b}, R², R³, R^{4a}, R^{4b} and R⁶ are as defined above.

More particularly, the present invention provides a compound of formula (IB-1), (IB-2), (IB-3) or (IB-4) as defined above or an N-oxide thereof, or a pharmaceutically acceptable salt thereof:

Typically, the present invention provides a compound of formula (IB-1), (IB-3), (IB-4) or (IB-5) as defined above or an N-oxide thereof, or a pharmaceutically acceptable salt thereof:

Suitably, the present invention provides a compound of formula (IB-3) as defined above or an N-oxide thereof, or a pharmaceutically acceptable salt thereof.

In a first embodiment, Y represents —O—. In a second embodiment, Y represents —N(R⁷)—. In a third embodiment, Y represents —C(R^{5a})(R^{5b})—. In a fourth embodiment, Y represents —S—. In a fifth embodiment, Y represents —S(O)—. In a sixth embodiment, Y represents —S(O)₂—. In a seventh embodiment, Y represents —S(O)(N—R⁸)—.

Typically, Y represents —O—, —N(R⁷)—, —C(R^{5a})(R^{5b})— or —S(O)₂—, wherein R^{5a}, R^{5b} and R⁷ are as defined above.

Appositely, Y represents —N(R⁷)— or —C(R^{5a})(R^{5b})—, wherein R^{5a}, R^{5b} and R⁷ are as defined above.

Suitably, Y represents —C(R^{5a})(R^{5b})—, wherein R^{5a} and R^{5b} are as defined above.

Generally, Z represents furyl, benzofuryl, dibenzofuryl, thienyl, benzothienyl, thieno[2,3-c]pyrazolyl, thieno[3,4-b][1,4]dioxinyl, dibenzothienyl, pyrrolyl, indolyl, pyrrolo[2,3-b]pyridinyl, pyrrolo[3,2-c]pyridinyl, pyrrolo[3,4-b]pyridinyl, pyrazolyl, pyrazolo[1,5-a]pyridinyl, 4,5,6,7-tetrahydropyrazolo[1,5-a]pyridinyl, pyrazolo[3,4-d]-pyrimidinyl, pyrazolo[1,5-a]pyrazinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo[2,1-b]thiazolyl, imidazo[1,2-a]pyridinyl, 5,6,7,8-tetrahydroimidazo[1,2-a]pyridinyl, imidazo[4,5-b]pyridinyl, imidazo[1,2-b]pyridazinyl, purinyl, imidazo[1,2-a]pyrimidinyl, imidazo[1,2-c]pyrimidinyl, imidazo[1,2-a]pyrazinyl, oxadiazolyl, thiadiazolyl, triazolyl, [1,2,4]triazolo[1,5-a]pyridinyl, [1,2,4]triazolo[4,3-a]-pyridinyl, [1,2,4]triazolo[4,3-a]pyrazinyl, 5,6,7,8-tetrahydro[1,2,4]triazolo[4,3-a]-pyridinyl, [1,2,4]triazolo[1,5-a]pyrimidinyl, 6,8-dihydro-5H-[1,2,4]triazolo[4,3-a]-pyrazinyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl or chromenyl, any of which groups may be optionally substituted by one or more substituents.

Appositely, Z represents pyrazolyl, pyrazolo[1,5-a]pyridinyl, isoxazolyl, isothiazolyl, imidazolyl, imidazo[1,2-a]pyridinyl, imidazo[1,2-a]pyrazinyl, oxadiazolyl, thiadiazolyl, triazolyl, [1,2,4]triazolo[1,5-a]pyridinyl, [1,2,4]triazolo[1,5-a]pyrazinyl, [1,2,4]triazolo[4,3-a]pyridinyl, tetrazolyl, pyridinyl, pyridazinyl, pyrimidinyl or pyrazinyl, any of which groups may be optionally substituted by one or more substituents.

Typically, Z represents imidazolyl, triazolyl, [1,2,4]triazolo[4,3-a]pyridinyl or tetrazolyl, any of which groups may be optionally substituted by one or more substituents.

More particularly, Z represents [1,2,4]triazolo[4,3-a]pyridinyl or tetrazolyl, either of which groups may be optionally substituted by one or more substituents.

Suitably, Z represents triazolyl, which group may be optionally substituted by one or more substituents.

Typical examples of optional substituents on Z include one, two or (where possible) three substituents independently selected from halogen, cyano, nitro, C₁₋₆ alkyl, difluoromethyl, difluoroethyl, trifluoro(C₁₋₆)alkyl, cyclopropyl, difluorocyclopropyl, difluorocyclobutyl, cyclopropylmethyl, difluorocyclopropylmethyl, fluorobicyclo[1.1.1]-pentanyl, cyanobicyclo[1.1.1]pentanyl, spiro[2.2]pentanyl, methylspiro[2.2]pentanyl, hydroxy, hydroxy(C₁₋₆)alkyl, oxo, C₁₋₆ alkoxy, difluoromethoxy, difluoroethoxy, trifluoromethoxy, trifluoroethoxy, phenoxy, methylenedioxy, difluoromethylenedioxy, C₁₋₆ alkylthio, C₁₋₆ alkylsulfinyl, C₁₋₆ alkylsulfonyl, amino, C₁₋₆ alkylamino, di(C₁₋₆)alkylamino, amino(C₁₋₆)alkyl, di(C₁₋₆)alkylamino(C₁₋₆)alkyl, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl and di(C₁₋₆)alkylsulfoximino.

Apposite examples of optional substituents on Z include one, two or (where possible) three substituents independently selected from halogen, cyano, C₁₋₆ alkyl, difluoromethyl, difluoroethyl, trifluoro(C₁₋₆)alkyl, cyclopropyl, difluorocyclopropyl, difluorocyclobutyl, cyclopropylmethyl, difluorocyclopropylmethyl, cyanobicyclo[1.1.1]-pentanyl and C₁₋₆ alkylamino.

Illustrative examples of optional substituents on Z include one, two or (where possible) three substituents independently selected from halogen and trifluoro(C₁₋₆)alkyl.

Suitable examples of optional substituents on Z include one, two or (where possible) three substituents independently selected from trifluoro(C₁₋₆)alkyl.

Typical examples of particular substituents on Z include one, two or (where possible) three substituents independently selected from fluoro, chloro, bromo, cyano, nitro, methyl, ethyl, n-propyl, isopropyl, tert-butyl, difluoromethyl, difluoroethyl, trifluoromethyl, trifluoroethyl, trifluoropropyl, 2-methyl-3,3,3-trifluoropropyl, cyclopropyl, difluorocyclopropyl, difluorocyclobutyl, cyclopropylmethyl, difluorocyclopropylmethyl, fluorobicyclo[1.1.1]pentanyl, cyanobicyclo[1.1.1]pentanyl, spiro-[2.2]pentanyl, methylspiro[2.2]pentanyl, hydroxy, hydroxymethyl, hydroxyethyl, hydroxyisopropyl, oxo, methoxy, isopropoxy, difluoromethoxy, difluoroethoxy, trifluoromethoxy, trifluoroethoxy, phenoxy, methylenedioxy, difluoromethylenedioxy, methylthio, methylsulfinyl, methylsulfonyl, amino, methylamino, dimethylamino, aminomethyl, dimethylaminomethyl, acetylamino, methoxycarbonylamino, methylsulfonylamino, formyl, acetyl, carboxy, methoxycarbonyl, ethoxycarbonyl, aminocarbonyl, methylaminocarbonyl, dimethylaminocarbonyl, aminosulfonyl, methylaminosulfonyl, dimethylaminosulfonyl and dimethylsulfoximino.

Apposite examples of particular substituents on Z include one, two or (where possible) three substituents independently selected from fluoro, cyano, methyl, difluoro-methyl, difluoroethyl, trifluoroethyl, trifluoropropyl, 2-methyl-3,3,3-trifluoropropyl, cyclopropyl, difluorocyclopropyl, difluorocyclobutyl, cyclopropylmethyl, difluorocyclopropylmethyl, cyanobicyclo[1.1.1]pentanyl and methylamino.

Illustrative examples of particular substituents on Z include one, two or (where possible) three substituents independently selected from fluoro and trifluoroethyl.

Suitable examples of particular substituents on Z include one, two or (where possible) three substituents independently selected from trifluoroethyl.

Typical values of Z include trifluoroethylpyrazolyl, (methyl)(trifluoroethyl)-pyrazolyl, pyrazolo[1,5-a]pyridinyl, methylindazolyl, trifluoroethylisoxazolyl, (methyl)-(trifluoroethyl)isoxazolyl, trifluoroethylisothiazolyl, trifluoroethylimidazolyl, cyclopropylmethylimidazolyl, (methyl)(trifluoroethyl)imidazolyl, imidazo[1,2-a]-pyridinyl, difluoroethyltriazolyl, trifluoroethyltriazolyl, difluorocyclopropyltriazolyl, difluorocyclobutyltriazolyl, cyclopropylmethyltriazolyl, cyanobicyclo[1.1.1]pentanyltriazolyl, (fluoro)(trifluoroethyl)triazolyl, (methyl)(trifluoroethyl)triazolyl, (difluoro-methyl)(trifluoroethyl)triazolyl, (cyclopropylmethyl)(difluoromethyl)triazolyl, (methyl-amino)(trifluoroethyl)triazolyl, [1,2,4]triazolo[1,5-a]pyridinyl, fluoro[1,2,4]triazolo[4,3-a]pyridinyl, cyano[1,2,4]triazolo[4,3-a]pyridinyl, benzotriazolyl, trifluoroethyltetrazolyl, trifluoroethylpyridinyl, trifluoroethylpyridazinyl, trifluoroethylpyrimidinyl and trifluoroethylpyrazinyl.

Illustrative values of Z include cyclopropylmethylimidazolyl, difluoroethyltriazolyl, trifluoroethyltriazolyl, difluorocyclobutyltriazolyl, cyclopropylmethyltriazolyl, cyanobicyclo[1.1.1]pentanyltriazolyl, fluoro[1,2,4]triazolo[4,3-a]pyridinyl, cyano[1,2,4]-triazolo[4,3-a]pyridinyl and trifluoroethyltetrazolyl.

Apposite values of Z include fluoro[1,2,4]triazolo[4,3-a]pyridinyl and trifluoroethyltetrazolyl.

Suitable values of Z include trifluoroethyltriazolyl.

Suitably, Z represents a group of formula (Za), (Zb), (Zc), (Zd), (Ze), (Zf), (Zg), (Zh), (Zj), (Zk), (Zl), (Zm), (Zn), (Zp), (Zq), (Zr), (Zs), (Zt), (Zu), (Zv), (Zw), (Zx), (Zy), (Zz), (Zaa) or (Zab):

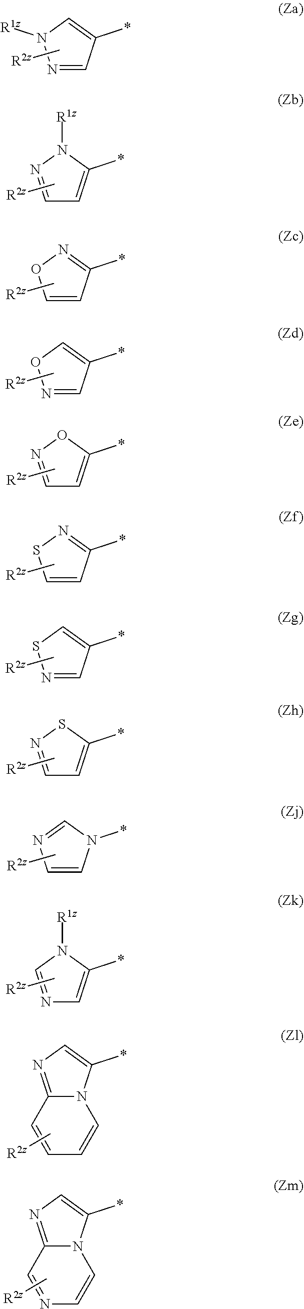

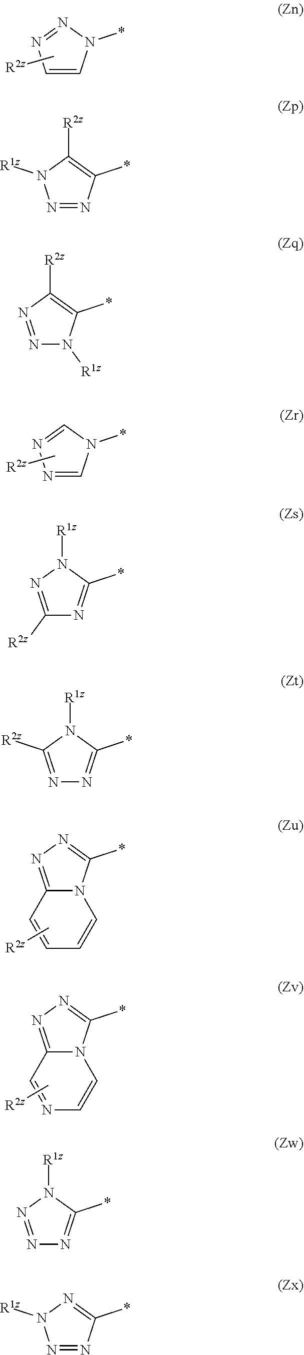

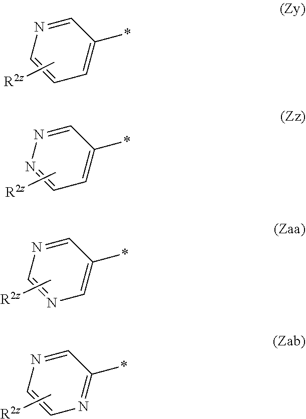

wherein
the asterisk (*) represents the point of attachment to the remainder of the molecule;
R^{1z} represents hydrogen, C₁₋₆ alkyl, difluoromethyl, difluoroethyl, trifluoro(C₁₋₆)-alkyl, cyclopropyl, difluorocyclopropyl, difluorocyclobutyl, cyclopropylmethyl, difluorocyclopropylmethyl, fluorobicyclo[1.1.1]pentanyl, cyanobicyclo[1.1.1]pentanyl, spiro[2.2]pentanyl, methylspiro[2.2]pentanyl, hydroxy(C₂₋₆)alkyl, C₁₋₆ alkylsulfonyl, amino(C₂₋₆)alkyl, di(C₁₋₆)alkylamino(C₁₋₆)alkyl, C₂₋₆ alkylcarbonyl, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl or di(C₁₋₆)alkylaminosulfonyl; and
R^{2z} represents hydrogen, halogen, cyano, nitro, C₁₋₆ alkyl, difluoromethyl, trifluoro(C₁₋₆)alkyl, cyclopropyl, difluorocyclopropyl, difluorocyclobutyl, cyclopropylmethyl, difluorocyclopropylmethyl, fluorobicyclo[1.1.1]pentanyl, cyanobicyclo[1.1.1]-pentanyl, spiro[2.2]pentanyl, methylspiro[2.2]pentanyl, hydroxy, hydroxy(C₁₋₆)alkyl, C₁₋₆ alkoxy, difluoromethoxy, difluoroethoxy, trifluoromethoxy, trifluoroethoxy, phenoxy, C₁₋₆ alkylthio, C₁₋₆ alkylsulfinyl, C₁₋₆ alkylsulfonyl, amino, C₁₋₆ alkylamino, di(C₁₋₆)alkylamino, amino(C₁₋₆)alkyl, di(C₁₋₆)alkylamino(C₁₋₆)alkyl, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl or di(C₁₋₆)alkylsulfoximino.

Particular values of Z include the groups of formula (Zk), (Zm), (Zp), (Zq), (Zt), (Zu), (Zv), (Zw) and (Zx) as defined above.

Suitable values of Z include the groups of formula (Zu) and (Zw) as defined above.

Appositely, Z represents the group of formula (Zq) as defined above.

Typically, R^{1z} represents hydrogen, C₁₋₆ alkyl, difluoroethyl, trifluoro(C₁₋₆)alkyl, difluorocyclopropyl, difluorocyclobutyl, cyclopropylmethyl, difluorocyclopropylmethyl or cyanobicyclo[1.1.1]pentanyl.

Apposite values of R^{1z} include hydrogen, methyl, ethyl, n-propyl, isopropyl, tert-butyl, difluoromethyl, trifluoromethyl, difluoroethyl, trifluoroethyl, trifluoropropyl, 2-methyl-3,3,3-trifluoropropyl, cyclopropyl, difluorocyclopropyl, difluorocyclobutyl, cyclopropylmethyl, difluorocyclopropylmethyl, fluorobicyclo[1.1.1]pentanyl, cyanobicyclo[1.1.1]pentanyl, spiro[2.2]pentanyl, methylspiro[2.2]pentanyl, hydroxyethyl, hydroxyisopropyl, methylsulfonyl, aminoethyl, dimethylaminomethyl, acetyl, methoxycarbonyl, ethoxycarbonyl, aminocarbonyl, methylaminocarbonyl, dimethylaminocarbonyl, aminosulfonyl, methylaminosulfonyl and dimethylaminosulfonyl.

Typical values of R^{1z} include hydrogen, methyl, ethyl, n-propyl, isopropyl, tert-butyl, difluoroethyl, trifluoroethyl, trifluoropropyl, 2-methyl-3,3,3-trifluoropropyl, difluorocyclopropyl, difluorocyclobutyl, cyclopropylmethyl, difluorocyclopropylmethyl and cyanobicyclo[1.1.1]pentanyl.

Illustrative values of R^{1z} include difluoroethyl, trifluoroethyl, difluorocyclobutyl, cyclopropylmethyl and cyanobicyclo[1.1.1]pentanyl.

Suitably, R^{1z} represents trifluoroethyl.

Typically, R^{2z} represents hydrogen, halogen, cyano, C₁₋₆ alkyl, trifluoro(C₁₋₆)alkyl, cyclopropylmethyl, difluorocyclopropylmethyl or C₁₋₆ alkylamino.

Suitably, R^{2z} represents hydrogen, halogen or cyano. In a first embodiment, R^{2z} represents hydrogen. In a second embodiment, R^{2z} represents halogen, especially fluoro. In a third embodiment, R^{2z} represents cyano.

Apposite values of R^{2z} include hydrogen, fluoro, chloro, bromo, cyano, nitro, methyl, ethyl, n-propyl, isopropyl, tert-butyl, difluoromethyl, trifluoromethyl, trifluoroethyl, trifluoropropyl, 2-methyl-3,3,3-trifluoropropyl, cyclopropyl, difluorocyclopropyl, difluorocyclobutyl, cyclopropylmethyl, difluorocyclopropylmethyl, fluorobicyclo[1.1.1]-pentanyl, cyanobicyclo[1.1.1]pentanyl, spiro[2.2]pentanyl, methylspiro[2.2]pentanyl, hydroxy, hydroxymethyl, hydroxyethyl, hydroxyisopropyl, methoxy, isopropoxy, difluoromethoxy, difluoroethoxy, trifluoromethoxy, trifluoroethoxy, phenoxy, methylthio, methylsulfinyl, methylsulfonyl, amino, methylamino, ethylamino, dimethylamino, aminomethyl, dimethylaminomethyl, acetylamino, methoxycarbonylamino, methylsulfonylamino, formyl, acetyl, carboxy, methoxycarbonyl, ethoxycarbonyl, aminocarbonyl, methylaminocarbonyl, dimethylaminocarbonyl, aminosulfonyl, methylaminosulfonyl, dimethylaminosulfonyl and dimethylsulfoximino.

Typical values of R^{2z} include hydrogen, fluoro, cyano, methyl, difluoromethyl, trifluoroethyl, trifluoropropyl, 2-methyl-3,3,3-trifluoropropyl, cyclopropylmethyl, difluorocyclopropylmethyl and methylamino.

Suitable values of R^{2z} include hydrogen, fluoro and cyano.

A particular value of R^{2z} is fluoro.

In a first embodiment, R^{1a} represents hydrogen. In a second embodiment, Ria represents fluoro. In a third embodiment, R^{1a} represents chloro. In a fourth embodiment, R^{1a} represents methyl. In a fifth embodiment, R^{1a} represents difluoromethyl. In a sixth embodiment, R^{1a} represents trifluoromethyl.

Typically, R^{1a} represents hydrogen, fluoro, chloro or methyl.

Generally, R^{1a} represents hydrogen or fluoro.

Suitably, R^{1a} represents hydrogen.

In a first embodiment, R^{1b} represents hydrogen. In a second embodiment, Rib represents fluoro. In a third embodiment, R^{1b} represents chloro. In a fourth embodiment, R^{1b} represents methyl. In a fifth embodiment, R^{1b} represents difluoromethyl. In a sixth embodiment, R^{1b} represents trifluoromethyl.

Typically, R^{1b} represents hydrogen, fluoro, chloro or methyl.

Generally, R^{1b} represents hydrogen or fluoro.

Suitably, R^{1b} represents hydrogen.

Suitably, R² represents C₃₋₉ cycloalkyl, C₄₋₁₂ bicycloalkyl or C₃₋₇ heterocycloalkyl, any of which groups may be optionally substituted by one or more substituents.

Appositely, R² represents C₃₋₉ cycloalkyl or C₄₋₁₂ bicycloalkyl, either of which groups may be optionally substituted by one or more substituents.

Typical examples of R² include cyclobutyl, bicyclo[1.1.1]pentanyl, azetidinyl, pyrrolidinyl, tetrahydropyranyl and morpholinyl, any of which groups may be optionally substituted by one or more substituents.

Apposite examples of R² include bicyclo[1.1.1]pentanyl and pyrrolidinyl, either of which groups may be optionally substituted by one or more substituents.

Suitable examples of R² include bicyclo[1.1.1]pentanyl, which group may be optionally substituted by one or more substituents.

Typical examples of optional substituents on R² include one, two, three or four substituents independently selected from halogen.

Typical examples of particular substituents on R² include one, two, three or four substituents independently selected from fluoro.

Typical values of R² include difluorocyclobutyl, fluorobicyclo[1.1.1]pentanyl, difluoroazetidinyl, difluoropyrrolidinyl, tetrafluoropyrrolidinyl, difluorotetrahydropyranyl and tetrafluoromorpholinyl.

Apposite values of R² include fluorobicyclo[1.1.1]pentanyl and tetrafluoropyrrolidinyl.

Suitable values of R² include fluorobicyclo[1.1.1]pentanyl.

In a first embodiment, R^{2a} represents C₁₋₆ alkyl. In a second embodiment, R^{2a} represents optionally substituted C₃₋₉ cycloalkyl.

Typically, R^{2a} represents C₁₋₆ alkyl; or R^{2a} represents cyclobutyl, which group may be optionally substituted by one or more substituents.

Typical examples of optional substituents on R^{2a} include one, two or three substituents independently selected from halogen, cyano, nitro, C₁₋₆ alkyl, trifluoromethyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, amino(C₁₋₆)alkyl, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Suitable examples of optional substituents on R^{2a} include one, two or three substituents independently selected from halogen.

Typical examples of specific substituents on R^{2a} include one, two or three substituents independently selected from fluoro, chloro, bromo, cyano, nitro, methyl, ethyl, isopropyl, tert-butyl, trifluoromethylhydroxy, hydroxymethyl, oxo, methoxy, tert-butoxy, difluoromethoxy, trifluoromethoxy, methylthio, methylsulfinyl, methylsulfonyl, amino, aminomethyl, aminoethyl, methylamino, tert-butylamino, dimethylamino, acetylamino, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl and dimethylaminosulfonyl.

Suitable examples of specific substituents on R^{2a} include one, two or three substituents independently selected from fluoro.

Illustrative examples of specific values of R^{2a} include methyl, ethyl, n-propyl, isopropyl, n-butyl, tert-butyl, cyclobutyl and difluorocyclobutyl.

In a first embodiment, R³ represents —NR^{3a}R^{3b}. In a second embodiment, R³ represents a group of formula (Wa) as defined above.

In a first embodiment, R^{3a} represents hydrogen. In a second embodiment, R^{3a} represents C₁₋₆ alkyl, especially methyl or ethyl. In a first aspect of that embodiment, R^{3a} represents methyl. In a second aspect of that embodiment, R^{3a} represents ethyl.

Typically, R^{3b} represents C₁₋₆ alkyl or C₃₋₇ cycloalkyl(C₁₋₆)alkyl, either of which groups may be optionally substituted by one or more substituents.

Suitably, R^{3b} represents C₁₋₆ alkyl, which group may be optionally substituted by one or more substituents.

In a first embodiment, R^{3b} represents optionally substituted C₁₋₆ alkyl. In a second embodiment, R^{3b} represents optionally substituted C₃₋₇ cycloalkyl. In a third embodiment, R^{3b} represents optionally substituted C₃₋₇ cycloalkyl(C₁₋₆)alkyl. In a fourth embodiment, R^{3b} represents optionally substituted C₄₋₁₂ bicycloalkyl. In a fifth embodiment, R^{3b} represents optionally substituted aryl. In a sixth embodiment, R^{3b} represents optionally substituted aryl(C₁₋₆)alkyl. In a seventh embodiment, R^{3b} represents optionally substituted C₃₋₇ heterocycloalkyl. In an eighth embodiment, R^{3b} represents optionally substituted C₃₋₇ heterocycloalkyl(C₁₋₆)alkyl. In a ninth embodiment, R^{3b} represents optionally substituted heteroaryl. In a tenth embodiment, R^{3b} represents optionally substituted heteroaryl(C₁₋₆)alkyl.

Apposite examples of R^{3b} include ethyl, propyl, isopropyl, 2-methylpropyl and cyclopropylmethyl, any of which groups may be optionally substituted by one or more substituents.

Typical examples of R^{3b} include ethyl, isopropyl, 2-methylpropyl and cyclopropylmethyl, any of which groups may be optionally substituted by one or more substituents.

Suitable examples of R^{3b} include ethyl, which group may be optionally substituted by one or more substituents.

Typical examples of optional substituents on R^{3b} include one, two or three substituents independently selected from halogen, cyano, nitro, C₁₋₆ alkyl, trifluoromethyl, hydroxy, C₁₋₆ alkoxy, difluoromethoxy, difluoroethoxy, trifluoromethoxy, trifluoroethoxy, C₁₋₆ alkylthio, C₁₋₆ alkylsulfinyl, C₁₋₆ alkylsulfonyl, amino, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl and di(C₁₋₆)alkylsulfoximino.

Apposite examples of optional substituents on R^{3b} include one, two or three substituents independently selected from halogen, trifluoromethyl and C₁₋₆ alkylaminocarbonyl.

Suitable examples of optional substituents on R^{3b} include one, two or three substituents independently selected from halogen.

Typical examples of particular substituents on R^{3b} include one, two or three substituents independently selected from fluoro, chloro, bromo, cyano, nitro, methyl, ethyl, trifluoromethyl, hydroxy, methoxy, isopropoxy, difluoromethoxy, difluoroethoxy, trifluoromethoxy, trifluoroethoxy, methylthio, methylsulfinyl, methylsulfonyl, ethylsulfonyl, amino, methylamino, dimethylamino, acetylamino, methoxycarbonylamino, methylsulfonylamino, formyl, acetyl, carboxy, methoxycarbonyl, ethoxycarbonyl, aminocarbonyl, methylaminocarbonyl, dimethylaminocarbonyl, aminosulfonyl, methylaminosulfonyl, dimethylaminosulfonyl and dimethylsulfoximino.

Apposite examples of particular substituents on R^{3b} include one, two or three substituents independently selected from fluoro, trifluoromethyl and methylaminocarbonyl.

Suitable examples of particular substituents on R^{3b} include one, two or three substituents independently selected from fluoro.

Typical values of R^{3b} include difluoroethyl, trifluoroethyl, trifluoroisopropyl, methylaminocarbonyl-2-methylpropyl, (cyclopropyl)(trifluoromethyl)methyl and difluorocyclopropylmethyl. Additional values include difluoropropyl.

Representative values of R^{3b} include trifluoroethyl, difluoropropyl, trifluoroisopropyl, methylaminocarbonyl-2-methylpropyl and (cyclopropyl)(trifluoromethyl)-methyl.

Apposite values of R^{3b} include trifluoroethyl, trifluoroisopropyl, methylaminocarbonyl-2-methylpropyl and (cyclopropyl)(trifluoromethyl)methyl.

Suitable values of R^{3b} include trifluoroethyl.

In a first embodiment, W represents the residue of an optionally substituted saturated monocyclic ring containing 3 to 6 carbon atoms, one nitrogen atom, and 0, 1, 2 or 3 additional heteroatoms independently selected from N, O and S, but containing no more than one O or S atom. In a first aspect of that embodiment, W represents the residue of an optionally substituted saturated monocyclic ring containing 3 or 4 carbon atoms, one nitrogen atom, and 0, 1, 2 or 3 additional heteroatoms independently selected from N, O and S, but containing no more than one O or S atom.

In a second embodiment, W represents the residue of an optionally substituted saturated bicyclic ring system containing 4 to 10 carbon atoms, one nitrogen atom, and 0, 1, 2 or 3 additional heteroatoms independently selected from N, O and S, but containing no more than one O or S atom. In a first aspect of that embodiment, W represents the residue of an optionally substituted saturated bicyclic ring system containing 5, 6 or 7 carbon atoms, one nitrogen atom, and 0, 1, 2 or 3 additional heteroatoms independently selected from N, O and S, but containing no more than one O or S atom.

In a third embodiment, W represents the residue of an optionally substituted saturated spirocyclic ring system containing 5 to 10 carbon atoms, one nitrogen atom, and 0, 1, 2 or 3 additional heteroatoms independently selected from N, O and S, but containing no more than one O or S atom. In a first aspect of that embodiment, W represents the residue of an optionally substituted saturated spirocyclic ring system containing 5, 6 or 7 carbon atoms, one nitrogen atom, and 0, 1, 2 or 3 additional heteroatoms independently selected from N, O and S, but containing no more than one O or S atom.

Suitably, W represents the residue of an optionally substituted saturated monocyclic ring containing 3 or 4 carbon atoms, one nitrogen atom, and 0 or 1 oxygen atom(s). In a first embodiment, W represents the residue of an optionally substituted saturated monocyclic ring containing 3 or 4 carbon atoms and one nitrogen atom. In a first aspect of that embodiment, W represents the residue of an optionally substituted saturated monocyclic ring containing 3 carbon atoms and one nitrogen atom. In a second aspect of that embodiment, W represents the residue of an optionally substituted saturated monocyclic ring containing 4 carbon atoms and one nitrogen atom. In a second embodiment, W represents the residue of an optionally substituted saturated monocyclic ring containing 4 carbon atoms, one nitrogen atom, and one oxygen atom.

In a first embodiment, the group of formula (Wa) represents a saturated monocyclic ring containing one nitrogen atom and no additional heteroatoms (i.e. it is an optionally substituted azetidin-1-yl, pyrrolidin-1-yl, piperidin-1-yl or hexahydroazepin-1-yl ring). In a second embodiment, the group of formula (Wa) represents a saturated monocyclic ring containing one nitrogen atom and one additional heteroatom selected from N, O and S. In a first aspect of that embodiment, the group of formula (Wa) is an optionally substituted morpholin-4-yl moiety. In a third embodiment, the group of formula (Wa) represents a saturated monocyclic ring containing one nitrogen atom and two additional heteroatoms selected from N, O and S, of which not more than one is O or S. In a fourth embodiment, the group of formula (Wa) represents a saturated monocyclic ring containing one nitrogen atom and three additional heteroatoms selected from N, O and S, of which not more than one is O or S.

Typical values of the group of formula (Wa) include azetidin-1-yl, pyrrolidin-1-yl, oxazolidin-3-yl, thiazolidin-3-yl, isothiazolidin-2-yl, imidazolidin-1-yl, piperidin-1-yl, piperazin-1-yl, homopiperazin-1-yl, morpholin-4-yl, thiomorpholin-4-yl, azepan-1-yl, [1,4]oxazepan-4-yl, [1,4]diazepan-1-yl, [1,4]thiadiazepan-4-yl, azocan-1-yl, 3-azabicyclo-[3.1.0]hexan-3-yl, 2-oxa-5-azabicyclo[2.2.1]heptan-5-yl, 6-azabicyclo[3.2.0]heptan-6-yl, 3-azabicyclo[3.1.1]heptan-3-yl, 6-oxa-3-azabicyclo[3.1.1]heptan-3-yl, 3-azabicyclo-[4.1.0]heptan-3-yl, 2-oxa-5-azabicyclo[2.2.2]octan-5-yl, 3-azabicyclo[3.2.1]octan-3-yl, 8-azabicyclo[3.2.1]octan-8-yl, 3-oxa-8-azabicyclo[3.2.1]octan-8-yl, 3,8-diazabicyclo-[3.2.1]octan-3-yl, 3,8-diazabicyclo[3.2.1]octan-8-yl, 3,6-diazabicyclo[3.2.2]nonan-3-yl, 3,6-diazabicyclo[3.2.2]nonan-6-yl, 3-oxa-7-azabicyclo[3.3.1]nonan-7-yl, 3,7-dioxa-9-azabicyclo[3.3.1]nonan-9-yl, 3,9-diazabicyclo[4.2.1]nonan-3-yl, 3,9-diazabicyclo[4.2.1]-nonan-9-yl, 5-azaspiro[2.3]hexan-5-yl, 5-azaspiro[2.4]heptan-5-yl, 2-azaspiro[3.3]heptan-2-yl, 2-oxa-6-azaspiro[3.3]heptan-6-yl, 3-oxa-6-azaspiro[3.3]heptan-6-yl, 6-thia-2-azaspiro[3.3]heptan-2-yl, 2-oxa-6-azaspiro[3.4]octan-6-yl, 2-oxa-6-azaspiro[3.5]nonan-6-yl, 7-oxa-2-azaspiro[3.5]nonan-2-yl, 2-oxa-7-azaspiro[3.5]nonan-7-yl, 2,4,8-triazaspiro[4.5]-decan-2-yl, 2,4,8-triazaspiro[4.5]decan-4-yl and 2,4,8-triazaspiro[4.5]decan-8-yl, any of which groups may be optionally substituted by one or more substituents.

In a first embodiment, the group of formula (Wa) is unsubstituted. In a second embodiment, the group of formula (Wa) is substituted by one or more substituents, typically by one to six substituents, suitably by two to four substituents. In a first aspect of that embodiment, the group of formula (Wa) is substituted by one substituent. In a second aspect of that embodiment, the group of formula (Wa) is substituted by two substituents. In a third aspect of that embodiment, the group of formula (Wa) is substituted by three substituents. In a fourth aspect of that embodiment, the group of formula (Wa) is substituted by four substituents. In a fifth aspect of that embodiment, the group of formula (Wa) is substituted by five substituents. In a sixth aspect of that embodiment, the group of formula (Wa) is substituted by six substituents.

Typical examples of optional substituents on the group of formula (Wa) include halogen, C₁₋₆ alkyl, trifluoromethyl, hydroxy, hydroxy(C₁₋₆)alkyl, C₁₋₆ alkoxy, difluoromethoxy, trifluoromethoxy, C₁₋₆ alkoxy(C₁₋₆)alkyl, C₁₋₆ alkylthio, C₁₋₆ alkylsulfonyl, cyano, oxo, formyl, C₂₋₆ alkylcarbonyl, carboxy, carboxy(C₁₋₆)alkyl, C₂₋₆ alkoxycarbonyl, C₂₋₆ alkoxycarbonyl(C₁₋₆)alkyl, amino, amino(C₁₋₆)alkyl, C₁₋₆ alkylamino, di(C₁₋₆)alkylamino, C₂₋₆ alkylcarbonylamino, C₂₋₆ alkoxycarbonylamino, C₁₋₆ alkylsulfonylamino, aminocarbonyl, C₁₋₆ alkylaminocarbonyl and di(C₁₋₆)alkylaminocarbonyl.

Suitable examples of optional substituents on the group of formula (Wa) include halogen.

Typical examples of particular substituents on the group of formula (Wa) include fluoro, chloro, bromo, methyl, ethyl, isopropyl, trifluoromethyl, hydroxy, hydroxymethyl, hydroxyethyl, methoxy, isopropoxy, difluoromethoxy, trifluoromethoxy, methoxymethyl, methylthio, ethylthio, methylsulfonyl, cyano, oxo, formyl, acetyl, ethylcarbonyl, tert-butylcarbonyl, carboxy, carboxymethyl, methoxycarbonyl, ethoxycarbonyl, tert-butoxycarbonyl, methoxycarbonylmethyl, ethoxycarbonylmethyl, amino, aminomethyl, methyl-amino, ethylamino, dimethylamino, acetylamino, tert-butoxycarbonylamino, methylsulfonylamino, aminocarbonyl, methylaminocarbonyl and dimethylaminocarbonyl.

Suitable examples of particular substituents on the group of formula (Wa) include fluoro.

Generally, R^{4a} represents hydrogen or fluoro; or R^{4a} represents C₁₋₆ alkyl, which group may be optionally substituted by one or more substituents.

Typically, R^{4a} represents hydrogen; or R^{4a} represents C₁₋₆ alkyl, which group may be optionally substituted by one or more substituents.

Suitably, R^{4a} represents C₁₋₆ alkyl, which group may be optionally substituted by one or more substituents.

In a first embodiment, R^{4a} represents hydrogen. In a second embodiment, R^{4a} represents fluoro. In a third embodiment, R^{4a} represents hydroxy. In a fourth embodiment, R^{4a} represents C₁₋₆ alkyl, especially methyl or ethyl, which group may be optionally substituted by one or more substituents. In a first aspect of that embodiment, R^{4a} represents optionally substituted methyl. In a second aspect of that embodiment, R^{4a} represents optionally substituted ethyl.

Typical examples of optional substituents on R^{4a} include one, two or three substituents independently selected from halogen, cyano, nitro, hydroxy, C₁₋₆ alkoxy, difluoromethoxy, difluoroethoxy, trifluoromethoxy, trifluoroethoxy, C₁₋₆ alkylthio, C₁₋₆ alkylsulfinyl, C₁₋₆ alkylsulfonyl, amino, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di-(C₁₋₆)alkylaminocarbonyl, aminosulfonyl, C₁₋₆ alkylaminosulfonyl, di(C₁₋₆)alkylaminosulfonyl and di(C₁₋₆)alkylsulfoximino.

Suitable examples of optional substituents on R^{4a} include one, two or three substituents independently selected from halogen.

Typical examples of particular substituents on R^{4a} include one, two or three substituents independently selected from fluoro, chloro, bromo, cyano, nitro, hydroxy, methoxy, isopropoxy, difluoromethoxy, difluoroethoxy, trifluoromethoxy, trifluoroethoxy, methylthio, methylsulfinyl, methylsulfonyl, ethylsulfonyl, amino, methylamino, dimethylamino, acetylamino, methoxycarbonylamino, methylsulfonylamino, formyl, acetyl, carboxy, methoxycarbonyl, ethoxycarbonyl, aminocarbonyl, methylaminocarbonyl, dimethylaminocarbonyl, aminosulfonyl, methylaminosulfonyl, dimethylaminosulfonyl and dimethylsulfoximino.

Suitable examples of particular substituents on R^{4a} include one, two or three substituents independently selected from fluoro.

Illustrative values of R^{4a} include hydrogen, fluoro, hydroxy, methyl, difluoroethyl and trifluoroethyl.

Typical values of R^{4a} include methyl, difluoroethyl and trifluoroethyl.

Suitable values of R^{4a} include difluoroethyl.

Generally, R^{4b} represents hydrogen, fluoro or C₁₋₆ alkyl.

In a first embodiment, R^{4b} represents hydrogen. In a second embodiment, R^{4b} represents fluoro. In a third embodiment, R^{4b} represents C₁₋₆ alkyl, especially methyl or ethyl, which group may be unsubstituted or substituted by one or more substituents. In a first aspect of that embodiment, R^{4b} represents unsubstituted methyl or substituted methyl. In a second aspect of that embodiment, R^{4b} represents unsubstituted ethyl or substituted ethyl.

Typical examples of optional substituents on R^{4b} include one, two or three substituents independently selected from halogen, cyano, nitro, hydroxy, C₁₋₆ alkoxy, difluoromethoxy, difluoroethoxy, trifluoromethoxy, trifluoroethoxy, C₁₋₆ alkylthio, C₁₋₆ alkylsulfinyl, C₁₋₆ alkylsulfonyl, amino, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di-(C₁₋₆)alkylaminocarbonyl, aminosulfonyl, C₁₋₆ alkylaminosulfonyl, di(C₁₋₆)alkylaminosulfonyl and di(C₁₋₆)alkylsulfoximino.

Typical examples of particular substituents on R^{4b} include one, two or three substituents independently selected from fluoro, chloro, bromo, cyano, nitro, hydroxy, methoxy, isopropoxy, difluoromethoxy, difluoroethoxy, trifluoromethoxy, trifluoroethoxy, methylthio, methylsulfinyl, methylsulfonyl, ethylsulfonyl, amino, methylamino, dimethylamino, acetylamino, methoxycarbonylamino, methylsulfonylamino, formyl, acetyl, carboxy, methoxycarbonyl, ethoxycarbonyl, aminocarbonyl, methylaminocarbonyl, dimethylaminocarbonyl, aminosulfonyl, methylaminosulfonyl, dimethylaminosulfonyl and dimethylsulfoximino.

Typical values of R^{4b} include hydrogen and fluoro.

Alternatively, R^{4a} and R^{4b} may together form an optionally substituted cyclic moiety. Thus, R^{4a} and R^{4b}, when taken together with the carbon atom to which they are both attached, may represent C₃₋₇ cycloalkyl or C₃₋₇ heterocycloalkyl, either of which groups may be unsubstituted, or substituted by one or more substituents, typically by one or two substituents.

In a first embodiment, R^{4a} and R^{4b}, when taken together with the carbon atom to which they are both attached, may suitably represent C₃₋₇ cycloalkyl, which group may be unsubstituted, or substituted by one or more substituents, typically by one or two substituents. As a general illustration of that embodiment, R^{4a} and R^{4b}, when taken together with the carbon atom to which they are both attached, may suitably represent cyclopropyl, cyclobutyl, cyclopentyl or cyclohexyl, any of which groups may be unsubstituted, or substituted by one or more substituents, typically by one or two substituents. As a particular illustration of that embodiment, R^{4a} and R^{4b}, when taken together with the carbon atom to which they are both attached, may suitably represent cyclobutyl or cyclohexyl, either of which groups may be unsubstituted, or substituted by one or more substituents, typically by one or two substituents. In a first aspect of that embodiment, R^{4a} and R^{4b}, when taken together with the carbon atom to which they are both attached, may suitably represent a cyclopropyl ring, which may be unsubstituted, or substituted by one or more substituents, typically by one or two substituents. In a second aspect of that embodiment, R^{4a} and R^{4b}, when taken together with the carbon atom to which they are both attached, may suitably represent a cyclobutyl ring, which may be unsubstituted, or substituted by one or more substituents, typically by one or two substituents. In a third aspect of that embodiment, R^{4a} and R^{4b}, when taken together with the carbon atom to which they are both attached, may suitably represent a cyclopentyl ring, which may be unsubstituted, or substituted by one or more substituents, typically by one or two substituents. In a fourth aspect of that embodiment, R^{4a} and R^{4b}, when taken together with the carbon atom to which they are both attached, may suitably represent a cyclohexyl ring, which may be unsubstituted, or substituted by one or more substituents, typically by one or two substituents.

In a second embodiment, R^{4a} and R^{4b}, when taken together with the carbon atom to which they are both attached, may suitably represent C₃₋₇ heterocycloalkyl, which group may be unsubstituted, or substituted by one or more substituents, typically by one or two substituents. As a general illustration of that embodiment, R^{4a} and R^{4b}, when taken together with the carbon atom to which they are both attached, may suitably represent oxetanyl, pyrrolidinyl, tetrahydropyranyl or piperidinyl, any of which groups may be unsubstituted, or substituted by one or more substituents, typically by one or two substituents. As a particular illustration of that embodiment, R^{4a} and R^{4b}, when taken together with the carbon atom to which they are both attached, may suitably represent pyrrolidinyl, tetrahydropyranyl or piperidinyl, any of which groups may be unsubstituted, or substituted by one or more substituents, typically by one or two substituents. In a first aspect of that embodiment, R^{4a} and R^{4b}, when taken together with the carbon atom to which they are both attached, may suitably represent an oxetanyl ring, which may be unsubstituted, or substituted by one or more substituents, typically by one or two substituents. In a second aspect of that embodiment, R^{4a} and R^{4b}, when taken together with the carbon atom to which they are both attached, may suitably represent a pyrrolidinyl ring, which may be unsubstituted, or substituted by one or more substituents, typically by one or two substituents. In a third aspect of that embodiment, R^{4a} and R^{4b}, when taken together with the carbon atom to which they are both attached, may suitably represent a tetrahydropyranyl ring, which may be unsubstituted, or substituted by one or more substituents, typically by one or two substituents. In a fourth aspect of that embodiment, R^{4a} and R^{4b}, when taken together with the carbon atom to which they are both attached, may suitably represent a piperidinyl ring, which may be unsubstituted, or substituted by one or more substituents, typically by one or two substituents.

Typically, R^{4a} and R^{4b}, when taken together with the carbon atom to which they are both attached, may represent cyclopropyl, cyclobutyl, cyclopentyl, cyclohexyl, oxetanyl, pyrrolidinyl, tetrahydropyranyl or piperidinyl, any of which groups may be unsubstituted, or substituted by one or more substituents, typically by one or two substituents.

Appositely, R^{4a} and R^{4b}, when taken together with the carbon atom to which they are both attached, may represent cyclohexyl or tetrahydropyranyl, either of which groups may be unsubstituted, or substituted by one or more substituents, typically by one or two substituents.

Suitably, R^{4a} and R^{4b}, when taken together with the carbon atom to which they are both attached, may represent cyclohexyl, which group may be unsubstituted, or substituted by one or more substituents, typically by one or two substituents.

Typical examples of optional substituents on the cyclic moiety formed by R^{4a} and R^{4b} include one, two or three substituents independently selected from C₁₋₆ alkyl, halogen, cyano, trifluoromethyl, trifluoroethyl, hydroxy, C₁₋₆ alkoxy, C₁₋₆ alkylthio, C₁₋₆ alkylsulfinyl, C₁₋₆ alkylsulfonyl, C₂₋₆ alkylcarbonyl, C₂₋₆ alkoxycarbonyl, amino, C₁₋₆ alkylamino and di(C₁₋₆)alkylamino.

Suitable examples of optional substituents on the cyclic moiety formed by R^{4a} and R^{4b} include one, two or three substituents independently selected from halogen.

Typical examples of particular substituents on the cyclic moiety formed by R^{4a} and R^{4b} include one, two or three substituents independently selected from methyl, fluoro, chloro, bromo, cyano, trifluoromethyl, trifluoroethyl, hydroxy, methoxy, methylthio, methylsulfinyl, methylsulfonyl, acetyl, methoxycarbonyl, ethoxycarbonyl, amino, methyl-amino and dimethylamino.

Suitable examples of particular substituents on the cyclic moiety formed by R^{4a} and R^{4b} include one, two or three substituents independently selected from fluoro.

Typical examples of the cyclic moiety formed by R^{4a} and R^{4b} include cyclopropyl, difluorocyclobutyl, cyclopentyl, difluorocyclohexyl, oxetanyl, methoxycarbonyl-pyrrolidinyl, tetrahydropyranyl, piperidinyl and methoxycarbonylpiperidinyl.

Representative examples of the cyclic moiety formed by R^{4a} and R^{4b} include difluorocyclohexyl and tetrahydropyranyl.

Suitable examples of the cyclic moiety formed by R^{4a} and R^{4b} include difluorocyclohexyl.

In a first embodiment, R^{5a} represents hydrogen. In a second embodiment, R^{5a} represents fluoro. In a third embodiment, R^{5a} represents methyl. In a fourth embodiment, R^{5a} represents difluoromethyl. In a fifth embodiment, R^{5a} represents trifluoromethyl.

Typically, R^{5a} represents hydrogen, fluoro or trifluoromethyl.

Suitably, R^{5a} represents fluoro or trifluoromethyl.

Appositely, R^{5a} represents fluoro.

In a first embodiment, R^{5b} represents hydrogen. In a second embodiment, R^{5b} represents fluoro. In a third embodiment, R^{5b} represents methyl. In a fourth embodiment, R^{5b} represents hydroxy.

Typically, R^{5b} represents hydrogen, fluoro or hydroxy.

Suitably, R^{5b} represents fluoro or hydroxy.

Appositely, R^{5b} represents fluoro.

Alternatively, R^{5a} and R b may together form a spiro linkage. Thus, R^{5a} and R^{5b}, when taken together with the carbon atom to which they are both attached, may represent cyclopropyl.

Typically, R⁶ represents —OR^{6a} or —NR^{6b}R^{6c}; or R⁶ represents C₁₋₆ alkyl, C₃₋₉ cycloalkyl, C₃₋₉ cycloalkyl(C₁₋₆)alkyl, aryl, aryl(C₁₋₆)alkyl, heteroaryl or heteroaryl-(C₁₋₆)alkyl, any of which groups may be optionally substituted by one or more substituents.

Appositely, R⁶ represents —OR^{6a} or —NR^{6b}R^{6c}; or R⁶ represents aryl or heteroaryl, either of which groups may be optionally substituted by one or more substituents.

Suitably, R⁶ represents —OR^{6a}; or R⁶ represents heteroaryl, which group may be optionally substituted by one or more substituents.

In a first embodiment, R⁶ represents optionally substituted C₁₋₆ alkyl. In a second embodiment, R⁶ represents optionally substituted C₃₋₉ cycloalkyl. In a third embodiment,
R⁶ represents optionally substituted C₃₋₉ cycloalkyl(C₁₋₆)alkyl. In a fourth embodiment,
R⁶ represents optionally substituted aryl. In a fifth embodiment, R⁶ represents optionally substituted aryl(C₁₋₆)alkyl. In a sixth embodiment, R⁶ represents optionally substituted C₃₋₇ heterocycloalkyl. In a seventh embodiment, R⁶ represents optionally substituted C₃₋₇ heterocycloalkyl(C₁₋₆)alkyl. In an eighth embodiment, R⁶ represents optionally substituted heteroaryl. In a ninth embodiment, R⁶ represents optionally substituted heteroaryl(C₁₋₆)alkyl. In a tenth embodiment, R⁶ represents —OR^{6a}. In an eleventh embodiment, R⁶ represents —NR^{6a}R^{6b}.

Typical examples of R⁶ include —OR^{6a} or —NR^{6a}R^{6b}; and methyl, ethyl, propyl, 2-methylpropyl, butyl, cyclopropyl, cyclobutyl, cyclohexyl, cyclohexylmethyl, phenyl, benzyl, phenylethyl, pyrazolyl, isoxazolyl, oxadiazolyl, triazolyl, pyridinyl, triazolylmethyl, benzotriazolylmethyl or pyridinylmethyl, any of which groups may be optionally substituted by one or more substituents.

Representative examples of R⁶ include —OR^{6a} or —NR^{6a}R^{6b}; and phenyl, pyrazolyl, isoxazolyl, oxadiazolyl or triazolyl, any of which groups may be optionally substituted by one or more substituents.

Illustrative examples of R⁶ include —OR^{6a}; and pyrazolyl, isoxazolyl, oxadiazolyl or triazolyl, any of which groups may be optionally substituted by one or more substituents.

Suitable examples of R⁶ include pyrazolyl, isoxazolyl, oxadiazolyl and triazolyl, any of which groups may be optionally substituted by one or more substituents.

Apposite examples of R⁶ include pyrazolyl and oxadiazolyl, either of which groups may be optionally substituted by one or more substituents.

Particular examples of R⁶ include oxadiazolyl, which group may be optionally substituted by one or more substituents.

Typical examples of optional substituents on R⁶ include one, two or three substituents independently selected from halogen, cyano, nitro, C₁₋₆ alkyl, trifluoromethyl, cyclopropyl, phenyl, fluorophenyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, amino(C₁₋₆)alkyl, C₁₋₆ alkylamino, di(C₁₋₆)alkylamino, pyrrolidinyl, tetrahydropyranyl, morpholinyl, piperazinyl, C₂₋₆ alkylcarbonylamino, C₂₋₆ alkylcarbonylamino-(C₁₋₆)alkyl, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl and di(C₁₋₆)alkylsulfoximinyl.

Apposite examples of optional substituents on R⁶ include one, two or three substituents independently selected from halogen, C₁₋₆ alkyl and cyclopropyl.

Suitable examples of optional substituents on R⁶ include one, two or three substituents independently selected from C₁₋₆ alkyl and cyclopropyl.

Typical examples of particular substituents on R⁶ include one, two or three substituents independently selected from fluoro, chloro, bromo, cyano, nitro, methyl, ethyl, isopropyl, tert-butyl, trifluoromethyl, cyclopropyl, phenyl, fluorophenyl, hydroxy, hydroxymethyl, oxo, methoxy, tert-butoxy, difluoromethoxy, trifluoromethoxy, methylthio, methylsulfinyl, methylsulfonyl, amino, aminomethyl, aminoethyl, methyl-amino, tert-butylamino, dimethylamino, pyrrolidinyl, tetrahydropyranyl, morpholinyl, piperazinyl, acetylamino, acetylaminoethyl, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl, dimethylaminosulfonyl and dimethylsulfoximinyl.

Apposite examples of particular substituents on R⁶ include one, two or three substituents independently selected from fluoro, methyl, ethyl, isopropyl and cyclopropyl.

Suitable examples of particular substituents on R⁶ include one, two or three substituents independently selected from methyl, ethyl, isopropyl and cyclopropyl.

Illustrative examples of particular values of R⁶ include methyl, difluoromethyl, methylsulfonylmethyl, aminomethyl, methylaminomethyl, difluoroethyl, carboxyethyl, difluoropropyl, 2-methylpropyl, butyl, cyanocyclopropyl, methylcyclopropyl, ethyl-cyclopropyl, dimethylcyclopropyl, trifluoromethylcyclopropyl, phenylcyclopropyl, fluorophenylcyclopropyl, hydroxycyclopropyl, aminocyclopropyl, cyclobutyl, trifluoromethylcyclobutyl, cyclohexyl, cyclohexylmethyl, phenyl, fluorophenyl, chloro-phenyl, cyanophenyl, methylphenyl, hydroxyphenyl, methylsulfonylphenyl, dimethyl-sulfoximinylphenyl, benzyl, fluorobenzyl, difluorobenzyl, chlorobenzyl, (chloro)(fluoro)-benzyl, dichlorobenzyl, (chloro)(difluoro)benzyl, bromobenzyl, cyanobenzyl, methyl-benzyl, dimethylbenzyl, trifluoromethylbenzyl, phenylbenzyl, hydroxybenzyl, hydroxymethylbenzyl, benzoyl, methoxybenzyl, dimethoxybenzyl, trifluoromethoxy-benzyl, methylsulfonylbenzyl, aminomethylbenzyl, aminoethylbenzyl, dimethylamino-benzyl, pyrrolidinylbenzyl, (dimethyl)(pyrrolidinyl)benzyl, morpholinylbenzyl, (dimethyl)(morpholinyl)benzyl, piperazinylbenzyl, acetylaminoethylbenzyl, phenylethyl, chlorophenylethyl, methylpyrazolyl, ethylpyrazolyl, isopropylpyrazolyl, (methyl)-(tetrahydropyranyl)pyrazolyl, methylisoxazolyl, ethylisoxazolyl, methyloxadiazolyl, ethyloxadiazolyl, cyclopropyloxadiazolyl, isopropyltriazolyl, pyridinyl, triazolylmethyl, benzotriazolylmethyl, pyridinylmethyl and aminopyridinylmethyl.

Favoured values of R⁶ include methylpyrazolyl, ethylpyrazolyl, isopropylpyrazolyl, methylisoxazolyl, ethylisoxazolyl, methyloxadiazolyl, ethyloxadiazolyl, cyclopropyloxadiazolyl and isopropyltriazolyl.

Typical values of R⁶ include isopropylpyrazolyl, ethylisoxazolyl, methyloxadiazolyl, ethyloxadiazolyl, cyclopropyloxadiazolyl and isopropyltriazolyl.

Selected values of R⁶ include methylpyrazolyl, ethylpyrazolyl, methyloxadiazolyl and ethyloxadiazolyl.

Particular examples of selected values of R⁶ include methyloxadiazolyl and ethyloxadiazolyl. In a first embodiment, R⁶ represents methyloxadiazolyl. In a second embodiment, R⁶ represents optionally substituted ethyloxadiazolyl.

Generally, R^{6a} represents C₁₋₆ alkyl; or R^{6a} represents C₃₋₉ cycloalkyl, which group may be optionally substituted by one or more substituents.

In a first embodiment, R^{6a} represents C₁₋₆ alkyl. In a second embodiment, R^{6a} represents optionally substituted C₃₋₉ cycloalkyl. In a third embodiment, R^{6a} represents optionally substituted C₃₋₇ heterocycloalkyl.

Appositely, R^{6a} represents C₁₋₆ alkyl; or R^{6a} represents cyclobutyl or oxetanyl, either of which groups may be optionally substituted by one or more substituents.

Typically, R^{6a} represents C₁₋₆ alkyl; or R^{6a} represents cyclobutyl, which group may be optionally substituted by one or more substituents.

Typical examples of optional substituents on R^{6a} include one, two or three substituents independently selected from halogen, cyano, nitro, C₁₋₆ alkyl, trifluoromethyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, amino(C₁₋₆)alkyl, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Suitable examples of optional substituents on R^{6a} include one, two or three substituents independently selected from halogen.

Typical examples of specific substituents on R^{6a} include one, two or three substituents independently selected from fluoro, chloro, bromo, cyano, nitro, methyl, ethyl, isopropyl, tert-butyl, trifluoromethylhydroxy, hydroxymethyl, oxo, methoxy, tert-butoxy, difluoromethoxy, trifluoromethoxy, methylthio, methylsulfinyl, methylsulfonyl, amino, aminomethyl, aminoethyl, methylamino, tert-butylamino, dimethylamino, acetylamino, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl and dimethylaminosulfonyl.

Suitable examples of specific substituents on R^{6a} include one, two or three substituents independently selected from fluoro.

Representative examples of specific values of R^{a} include methyl, ethyl, n-propyl, isopropyl, n-butyl, tert-butyl, cyclobutyl, difluorocyclobutyl and oxetanyl.

Illustrative examples of specific values of R⁶ include methyl, ethyl, n-propyl, isopropyl, n-butyl, tert-butyl, cyclobutyl and difluorocyclobutyl.

Typically, R^{6a} represents cyclobutyl.

Typically, R^{b} represents hydrogen or methyl.

In a first embodiment, R^{6b} represents hydrogen. In a second embodiment, R^{6b} represents C₁₋₆ alkyl, especially methyl.

Typically, R^{6c} represents hydrogen or methyl.

In a first embodiment, R^{6c} represents hydrogen. In a second embodiment, R^{6c} represents C₁₋₆ alkyl, especially methyl.

Alternatively, the moiety —NR^{6b}R^{6c} may suitably represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents.

Selected examples of suitable substituents on the heterocyclic moiety —NR^{6b}R^{6c} include C₁₋₆ alkyl, C₁₋₆ alkylsulfonyl, hydroxy, hydroxy(C₁₋₆)alkyl, amino(C₁₋₆)alkyl, cyano, oxo, C₂₋₆ alkylcarbonyl, carboxy, C₂₋₆ alkoxycarbonyl, amino, C₂₋₆ alkylcarbonylamino, C₂₋₆ alkylcarbonylamino(C₁₋₆)alkyl, C₂₋₆ alkoxycarbonylamino, C₁₋₆ alkylsulfonylamino and aminocarbonyl.

Selected examples of specific substituents on the heterocyclic moiety —NR^{6b}R⁶, include methyl, methylsulfonyl, hydroxy, hydroxymethyl, aminomethyl, cyano, oxo, acetyl, carboxy, ethoxycarbonyl, amino, acetylamino, acetylaminomethyl, tert-butoxycarbonylamino, methylsulfonylamino and aminocarbonyl.

Generally, R⁷ represents —COR^{7a}, —CO₂R^{7a} or —SO₂R^{7b}; or R⁷ represents hydrogen; or R⁷ represents C₁₋₆ alkyl or C₃₋₉ cycloalkyl, either of which groups may be optionally substituted by one or more fluorine atoms.

More particularly, R⁷ represents —COR^{7a}, —CO₂R^{7a} or —SO₂R^{7b}; or R⁷ represents hydrogen; or R⁷ represents C₁₋₆ alkyl, which group may be optionally substituted by one or more fluorine atoms, generally by one, two or three fluorine atoms, typically by two fluorine atoms.

Typically, R⁷ represents C₃₋₇ heterocycloalkyl, which group may be optionally substituted by one or more fluorine atoms.

Suitably, R⁷ represents —CO₂R^{7a}.

In a first embodiment, R⁷ represents —COR^{7a}. In a second embodiment, R⁷ represents —CO₂R^{7a}. In a third embodiment, R⁷ represents —CO₂R^{7a}. In a fourth embodiment, R⁷ represents hydrogen. In a fifth embodiment, R⁷ represents C₁₋₆ alkyl, optionally substituted by one or more fluorine atoms, typically by one, two or three fluorine atoms. In one aspect of that embodiment, R⁷ represents unsubstituted C₁₋₆ alkyl, especially methyl or ethyl. In another aspect of that embodiment, R⁷ represents C₁₋₆ alkyl substituted by one, two or three fluorine atoms, typically by two fluorine atoms. Examples of that aspect include difluoroethyl. In a sixth embodiment, R⁷ represents C₃₋₉ cycloalkyl, optionally substituted by one or more fluorine atoms, typically by one, two or three fluorine atoms. In one aspect of that embodiment, R⁷ represents unsubstituted C₃₋₉ cycloalkyl, especially cyclopropyl, cyclobutyl, cyclopentyl or cyclohexyl. In another aspect of that embodiment, R⁷ represents C₃₋₉ cycloalkyl substituted by one, two or three fluorine atoms, typically by two fluorine atoms. Examples of that aspect include difluorocyclobutyl. In a seventh embodiment, R⁷ represents C₃₋₇ heterocycloalkyl, optionally substituted by one or more fluorine atoms, typically by one, two or three fluorine atoms. In one aspect of that embodiment, R⁷ represents unsubstituted C₃₋₇ heterocycloalkyl, especially oxetanyl. In another aspect of that embodiment, R⁷ represents C₃₋₇ heterocycloalkyl substituted by one, two or three fluorine atoms, typically by two fluorine atoms.

Particular values of R⁷ include oxetanyl.

Typically, R^{7a} represents C₁₋₆ alkyl, optionally substituted by one, two or three fluorine atoms.

Suitably, R^{7a} represents C₁₋₆ alkyl or difluoro(C₁₋₆)alkyl.

In a first embodiment, R^{7a} represents C₁₋₆ alkyl, especially methyl or ethyl. In a first aspect of that embodiment, R^{7a} represents methyl. In a second aspect of that embodiment, R^{7a} represents ethyl. In a second embodiment, R^{7a} represents difluoro(C₁₋₆)-alkyl, especially difluoroethyl.

Particular values of R^{7a} include methyl and difluoroethyl.

Suitably, R^{7b} represents methyl or ethyl. In a first embodiment, R^{7b} represents methyl. In a second embodiment, R^{7b} represents ethyl.

Suitably, R⁸ represents methyl or ethyl. In a first embodiment, R⁸ represents methyl. In a second embodiment, R⁸ represents ethyl.

Various sub-classes of compounds according to the invention are represented by the compounds of formula (IIA-1), (IIA-2), (IIB-1) and (IIB-2) and N-oxides thereof, and pharmaceutically acceptable salts thereof:

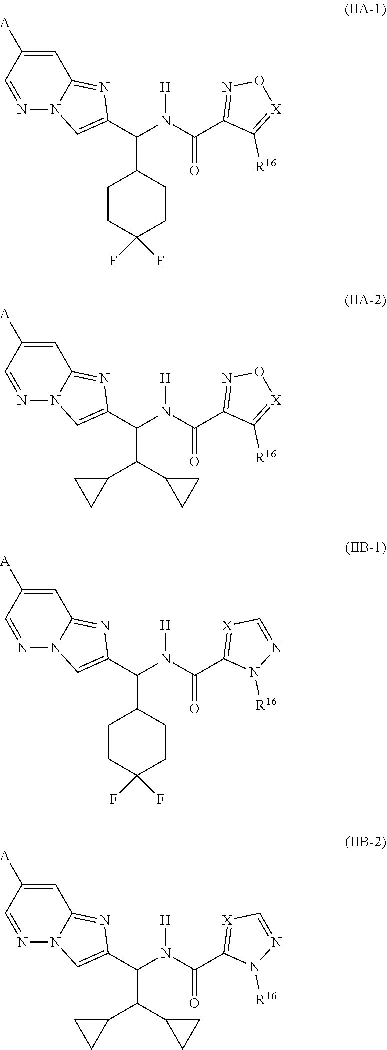

wherein
X represents CH or N;
R¹⁶ represents methyl, ethyl, isopropyl or cyclopropyl; and
A is as defined above.

In a first embodiment, X represents CH. In a second embodiment, X represents N.

In a first embodiment, R¹⁶ represents methyl. In a second embodiment, R¹⁶ represents ethyl. In a third embodiment, R¹⁶ represents isopropyl. In a fourth embodiment, R¹⁶ represents cyclopropyl.

Typically, R¹⁶ represents methyl, ethyl or cyclopropyl.

Suitably, R¹⁶ represents methyl or ethyl.

Specific novel compounds in accordance with the present invention include each of the compounds whose preparation is described in the accompanying Examples, and pharmaceutically acceptable salts and solvates thereof.

The compounds in accordance with the present invention are beneficial in the treatment and/or prevention of various human ailments, including inflammatory and autoimmune disorders.

The compounds according to the present invention are useful in the treatment and/or prophylaxis of a pathological disorder that is mediated by a pro-inflammatory IL-17 cytokine or is associated with an increased level of a pro-inflammatory IL-17 cytokine. Generally, the pathological condition is selected from the group consisting of infections (viral, bacterial, fungal and parasitic), endotoxic shock associated with infection, arthritis, rheumatoid arthritis, psoriatic arthritis, systemic onset juvenile idiopathic arthritis (JIA), systemic lupus erythematosus (SLE), asthma, chronic obstructive airways disease (COAD), chronic obstructive pulmonary disease (COPD), acute lung injury, pelvic inflammatory disease, Alzheimer's Disease, Crohn's disease, inflammatory bowel disease, irritable bowel syndrome, ulcerative colitis, Castleman's disease, axial spondyloarthritis, ankylosing spondylitis and other spondyloarthropathies, dermatomyositis, myocarditis, uveitis, exophthalmos, autoimmune thyroiditis, Peyronie's Disease, coeliac disease, gall bladder disease, Pilonidal disease, peritonitis, psoriasis, atopic dermatitis, hidradenitis suppurativa, vasculitis, surgical adhesions, stroke, autoimmune diabetes, Type I Diabetes, lyme arthritis, meningoencephalitis, immune mediated inflammatory disorders of the central and peripheral nervous system such as multiple sclerosis and Guillain-Barr syndrome, other autoimmune disorders, pancreatitis, trauma (surgery), graft-versus-host disease, transplant rejection, fibrosing disorders including pulmonary fibrosis, liver fibrosis, renal fibrosis, scleroderma or systemic sclerosis, cancer (both solid tumours such as melanomas, hepatoblastomas, sarcomas, squamous cell carcinomas, transitional cell cancers, ovarian cancers and hematologic malignancies and in particular acute myelogenous leukaemia, chronic myelogenous leukemia, chronic lymphatic leukemia, gastric cancer and colon cancer), heart disease including ischaemic diseases such as myocardial infarction as well as atherosclerosis, intravascular coagulation, bone resorption, osteoporosis, periodontitis, hypochlorhydia and pain (particularly pain associated with inflammation).

WO 2009/089036 reveals that modulators of IL-17 activity may be administered to inhibit or reduce the severity of ocular inflammatory disorders, in particular ocular surface inflammatory disorders including Dry Eye Syndrome (DES). Consequently, the compounds in accordance with the present invention are useful in the treatment and/or prevention of an IL-17-mediated ocular inflammatory disorder, in particular an IL-17-mediated ocular surface inflammatory disorder including Dry Eye Syndrome. Ocular surface inflammatory disorders include Dry Eye Syndrome, penetrating keratoplasty, corneal transplantation, lamellar or partial thickness transplantation, selective endothelial transplantation, corneal neovascularization, keratoprosthesis surgery, corneal ocular surface inflammatory conditions, conjunctival scarring disorders, ocular autoimmune conditions, Pemphigoid syndrome, Stevens-Johnson syndrome, ocular allergy, severe allergic (atopic) eye disease, conjunctivitis and microbial keratitis. Particular categories of Dry Eye Syndrome include keratoconjunctivitis sicca (KCS), Sjögren syndrome, Sjögren syndrome-associated keratoconjunctivitis sicca, non-Sjögren syndrome-associated keratoconjunctivitis sicca, keratitis sicca, sicca syndrome, xerophthalmia, tear film disorder, decreased tear production, aqueous tear deficiency (ATD), meibomian gland dysfunction and evaporative loss.

Illustratively, the compounds of the present invention may be useful in the treatment and/or prophylaxis of a pathological disorder selected from the group consisting of arthritis, rheumatoid arthritis, psoriasis, psoriatic arthritis, systemic onset juvenile idiopathic arthritis (JIA), systemic lupus erythematosus (SLE), asthma, chronic obstructive airway disease, chronic obstructive pulmonary disease, atopic dermatitis, hidradenitis suppurativa, scleroderma, systemic sclerosis, lung fibrosis, inflammatory bowel diseases (including Crohn's disease and ulcerative colitis), axial spondyloarthritis, ankylosing spondylitis and other spondyloarthropathies, cancer and pain (particularly pain associated with inflammation).

Suitably, the compounds of the present invention are useful in the treatment and/or prophylaxis of psoriasis, psoriatic arthritis, hidradenitis suppurativa, axial spondyloarthritis or ankylosing spondylitis.

The present invention also provides a pharmaceutical composition which comprises a compound in accordance with the invention as described above, or a pharmaceutically acceptable salt thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds according to the present invention may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents.

Alternatively, the active ingredient may be in powder form for constitution with a suitable vehicle, e.g. sterile pyrogen-free water, before use.

In addition to the formulations described above, the compounds according to the present invention may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds according to the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds according to the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds according to the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration the compounds according to the present invention may be formulated in an ointment such as petrolatum.

For rectal administration the compounds according to the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound according to the present invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

If desired, a compound in accordance with the present invention may be co-administered with another pharmaceutically active agent, e.g. an anti-inflammatory molecule.

The compounds of formula (I) above may be prepared by a process which comprises reacting a carboxylic acid of formula R⁶—CO₂H with a compound of formula (III):

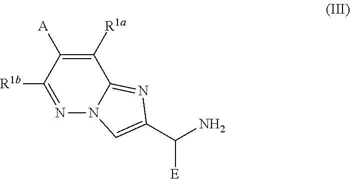

wherein A, E, R^{1a}, R^{1b} and R⁶ are as defined above.

The reaction is conveniently accomplished in the presence of a coupling agent and a base. Suitable coupling agents include 1-[bis(dimethylamino)methylene]-1H-1,2,3-triazolo[4,5-b]pyridinium 3-oxid hexafluorophosphate (HATU); and 2,4,6-tripropyl-1,3,5,2,4,6-trioxatriphosphorinane-2,4,6-trioxide. Suitable bases include organic amines, e.g. a trialkylamine such as N,N-diisopropylethylamine; or pyridine. The reaction is conveniently performed at ambient or elevated temperature in a suitable solvent, e.g. a cyclic ether such as tetrahydrofuran; or a dipolar aprotic solvent such as N,N-dimethylformamide or N,N-dimethylacetamide; or a chlorinated solvent such as dichloromethane; or an organic ester solvent such as ethyl acetate.

Alternatively, the reaction may conveniently be accomplished in the presence of a coupling agent such as N-(3-dimethylaminopropyl)-N′-ethylcarbodiimide (EDC). The reaction is suitably performed at an appropriate temperature, e.g. a temperature in the region of 0° C., in a suitable solvent, e.g. an organic nitrile solvent such as acetonitrile.

Where R⁶ represents C₁₋₆ alkyl, e.g. methyl, the compounds of formula (I) above may be prepared by a process which comprises reacting a compound of formula R⁶—COCl, e.g. acetyl chloride, with a compound of formula (III) as defined above. The reaction is conveniently accomplished in the presence of a base. Suitable bases include organic amines, e.g. a trialkylamine such as N,N-diisopropylethylamine. The reaction is conveniently performed at ambient temperature in a suitable solvent, e.g. a cyclic ether such as tetrahydrofuran.

Where R⁶ represents —OR^{6a}, the compounds of formula (I) above may be prepared by a two-step process which comprises: (i) reacting a compound of formula R^{6a}—OH with N,N′-disuccinimidyl carbonate, ideally in the presence of a base, e.g. an organic amine such as triethylamine; and (ii) reacting the resulting material with a compound of formula (III) as defined above. Steps (i) and (ii) are conveniently performed at ambient temperature in a suitable solvent, e.g. a chlorinated solvent such as dichloromethane, or an organic nitrile solvent such as acetonitrile.

The intermediates of formula (III) above may be prepared by removal of the N-protecting group R^{p} from a compound of formula (IV):

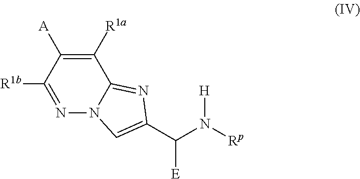

wherein A, E, R^{1a} and R^{1b} are as defined above, and R^{p} represents a N-protecting group.

The N-protecting group R^{p} will suitably be tert-butoxycarbonyl (BOC), in which case the removal thereof may conveniently be effected by treatment with an acid, e.g. a mineral acid such as hydrochloric acid, or an organic acid such as trifluoroacetic acid.

Alternatively, the N-protecting group R^{p} may be benzyloxycarbonyl, in which case the removal thereof may conveniently be effected by catalytic hydrogenation, typically by treatment with hydrogen gas or ammonium formate in the presence of a hydrogenation catalyst, e.g. palladium on charcoal, or palladium hydroxide on charcoal. In a variant method, where the N-protecting group R^{p} is benzyloxycarbonyl, the removal thereof may be effected by treatment with boron tribromide.

In an alternative procedure, the compounds of formula (I) above wherein A represents a group of formula (Ac) may be prepared by a two-step process which comprises:
(i) saponifying a compound of formula (V):

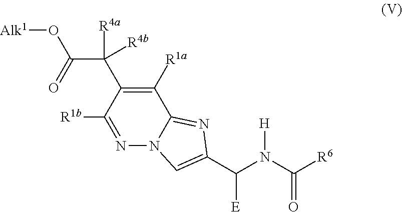

wherein E, R^{1a}, R^{1b}, R^{4a}, R^{4b} and R⁶ are as defined above, and Alk¹ represents C₁₋₄ alkyl, e.g. methyl, ethyl or tert-butyl; and
(ii) reaction of the carboxylic acid derivative thereby obtained with a compound of formula R³—H; under conditions analogous to those described above for the reaction between compound (III) and a carboxylic acid of formula R⁶—CO₂H.

Similarly, the intermediates of formula (IV) above wherein A represents a group of formula (Ac) may be prepared by a two-step process which comprises:
(i) saponifying a compound of formula (VI):

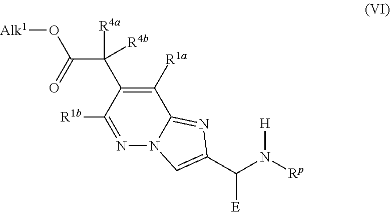

wherein E, R^{1a}, R^{1b}, R^{4a}, R^{4b}, R^{p} and Alk¹ are as defined above; and
(ii) reaction of the carboxylic acid derivative thereby obtained with a compound of formula R³—H; under conditions analogous to those described above for the reaction between compound (III) and a carboxylic acid of formula R⁶—CO₂H.

Where Alk¹ represents methyl or ethyl, the saponification reaction in step (i) will generally be effected by treatment with a base. Suitable bases include inorganic hydroxides, e.g. an alkali metal hydroxide such as lithium hydroxide or sodium hydroxide. The reaction is conveniently performed at ambient or elevated temperature in water and a suitable organic solvent, e.g. a cyclic ether such as tetrahydrofuran, or a C₁₋₄ alkanol such as methanol.

Alternatively, where Alk¹ represents tert-butyl, the saponification reaction in step (i) may generally be effected by treatment with an acid, e.g. an organic acid such as trifluoroacetic acid. The reaction is conveniently performed at ambient temperature in a suitable organic solvent, e.g. a chlorinated solvent such as dichloromethane.

Alternative coupling agents that may usefully be employed in step (ii) include 2-chloro-1-methylpyridinium iodide.

The intermediates of formula (V) above may be prepared by reacting a carboxylic acid of formula R⁶—CO₂H with a compound of formula (VII):

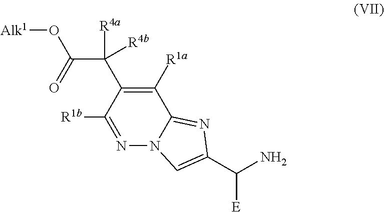

wherein E, R^{1a}, R^{1b}, R^{4a}, R^{4b}, R⁶ and Alk¹ are as defined above; under conditions analogous to those described above for the reaction between compound (III) and a carboxylic acid of formula R⁶—CO₂H.

The intermediates of formula (VII) above may be prepared by removal of the N-protecting group R^{p} from a compound of formula (VI) as defined above; under conditions analogous to those described above for the removal of the N-protecting group R^{p} from a compound of formula (IV).

The intermediates of formula (VI) above may be prepared by reacting a compound of formula (VIII) with a compound of formula (IX):

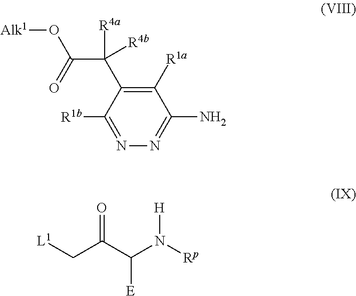

wherein E, R^{1a}, R^{1b}, R^{4a}, R^{4b}, Alk¹ and R^{p} are as defined above, and L¹ represents a suitable leaving group.

The leaving group L¹ is typically a halogen atom, e.g. bromo.

The reaction is typically accomplished in the presence of a base. Suitably, the base may be an inorganic base, e.g. a bicarbonate salt such as sodium bicarbonate; or an organic base such as pyridine. The reaction is conveniently effected at an elevated temperature in a suitable solvent, e.g. a C₁₋₄ alkanol such as isopropanol, or a cyclic ether such as 1,4-dioxane.

In an alternative procedure, the compounds of formula (I) above wherein A represents a group of formula (Aa) or (Ab) may be prepared by a process which comprises reacting a carboxylic acid of formula R²—CO₂H with a compound of formula (X):

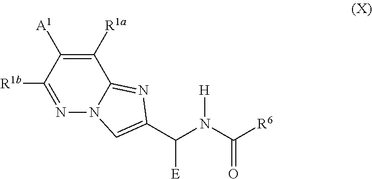

wherein
A¹ represents a group of formula (Aa-1) or (Ab-1):

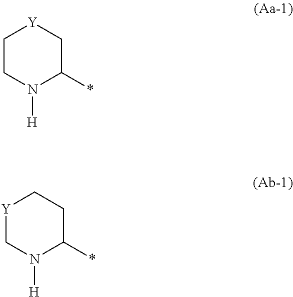

in which the asterisk (*) represents the point of attachment to the remainder of the molecule; and
E, Y, R^{1a}, R^{1b}, R² and R⁶ are as defined above;
   under conditions analogous to those described above for the reaction between compound (III) and a carboxylic acid of formula R⁶—CO₂H.

The intermediates of formula (X) above may be prepared by removal of the N-protecting group R^{z} from a compound of formula (XI):

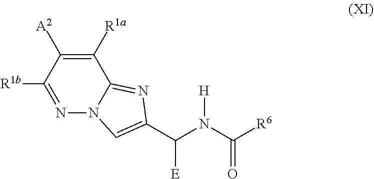

wherein
A² represents a group of formula (Aa-2) or (Ab-2):

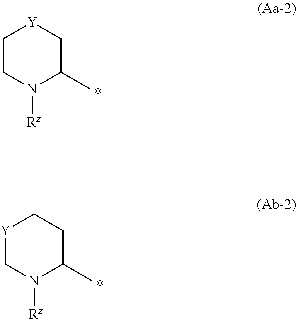

in which the asterisk (*) represents the point of attachment to the remainder of the molecule;
R^{z} represents a N-protecting group; and
E, Y, R^{1a}, R^{1b} and R⁶ are as defined above.

The N-protecting group R^{z} will suitably be tert-butoxycarbonyl (BOC), in which case the removal thereof may conveniently be effected by treatment with an acid, e.g. a mineral acid such as hydrochloric acid, or an organic acid such as trifluoroacetic acid.

The intermediates of formula (XI) above may be prepared by a two-step procedure which comprises the following steps:
(i) removal of the N-protecting group R^{p} from a compound of formula (XII):

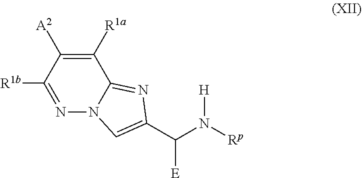

wherein E, A², R^{1a}, R^{1b} and R^{p} are as defined above; under conditions analogous to those described above; and
(ii) reaction of the material thereby obtained with a carboxylic acid of formula R⁶—CO₂H, under conditions analogous to those described above for the reaction between compound (III) and a carboxylic acid of formula R⁶—CO₂H.

The intermediates of formula (XII) above may be prepared by reacting a compound of formula A²-CO₂H with a compound of formula (XIII):

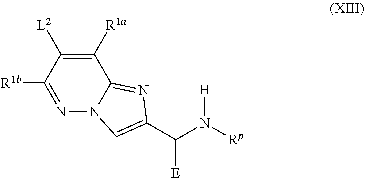

wherein E, A², R^{1a}, R^{1b} and R^{p} are as defined above, and L² represents a suitable leaving group; in the presence of a transition metal catalyst.

The leaving group L² is suitably a halogen atom, e.g. chloro or bromo.

Suitable transition metal catalysts of use in the reaction include [4,4′-bis(1,1-dimethylethyl)-2,2′-bipyridine-N1,N1′]bis-{3,5-difluoro-2-[5-(trifluoromethyl)-2-pyridinyl-N]phenyl-C}iridium(III) hexafluorophosphate. The reaction will generally be performed in the presence of nickel(II) chloride ethylene glycol dimethyl ether complex and 4,4′-di-tert-butyl-2,2′-dipyridyl. The reaction will suitably be effected in the presence of a base, e.g. an organic base such as 1,8-diazabicyclo[5.4.0]undec-7-ene, or an inorganic base such as cesium carbonate; and the reactants will typically be exposed to a bright light source. A suitable bright light source will typically comprise the ‘integrated photoreactor’ described in ACS Cent. Sci., 2017, 3, 647-653. The reaction will conveniently be carried out at ambient temperature in a suitable solvent, e.g. a dipolar aprotic solvent such as N,N-dimethylformamide.

The intermediates of formula (XIII) above may be prepared by reacting a compound of formula (IX) as defined above with a compound of formula (XIV):

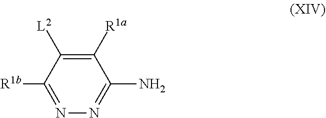

wherein R^{1a}, R^{1b} and L² are as defined above; under conditions analogous to those described above for the reaction between compounds (VIII) and (IX).

Alternatively, the intermediates of formula (XII) above may be prepared by reacting a compound of formula (IX) as defined above with a compound of formula (VIIIA):

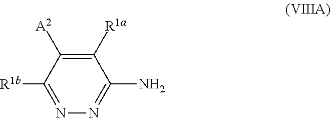

wherein A², R^{1a} and R^{1b} are as defined above; under conditions analogous to those described above for the reaction between compounds (VIII) and (IX).

The intermediates of formula (VIIIA) above may be prepared by reacting a compound of formula A²-CO₂H with a compound of formula (XIV) as defined above; under conditions analogous to those described above for the reaction between compound (XIII) and a compound of formula A²-CO₂H.

In an alternative procedure, the compounds of formula (I) wherein A represents a group of formula (Ad) and Z represents a group of formula (Zt) as defined above, in which R^{2z} is hydrogen, may be prepared by a process which comprises reacting a compound of formula R^{1z}—NH₂ and a trialkyl orthoformate HC(O-Alk¹)₃ with a compound of formula (XV):

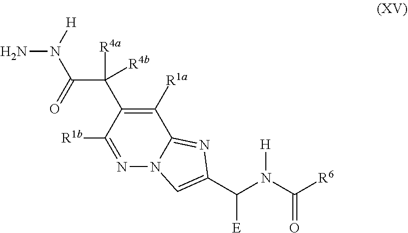

wherein E, R^{1a}, R^{1b}, R^{4a}, R^{4b}, R⁶, R^{1z} and Alk¹ are as defined above.

The reaction is conveniently performed at an elevated temperature in the presence of acetic acid. The reaction may typically be carried out in a suitable solvent, e.g. a cyclic ether such as 1,4-dioxane.

The intermediates of formula (XV) above may be prepared by reacting a compound of formula (V) as defined above with hydrazine hydrate.

The reaction is conveniently performed at an elevated temperature in a suitable solvent, e.g. a C₁₋₄ alkanol such as ethanol.

The intermediates of formula (IV) above wherein A represents a group of formula (Ad) and Z represents a group of formula (Zu) as defined above may be prepared by a three-step procedure which comprises the following steps:
(i) saponification of a compound of formula (VI) as defined above by treatment with a base;
(ii) reaction of the carboxylic acid derivative thereby obtained with a compound of formula (XVI):

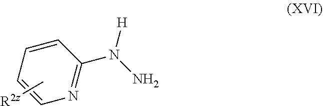

wherein R^{2z} is as defined above; under conditions analogous to those described above for the reaction between compound (III) and a carboxylic acid of formula R⁶—CO₂H; and (iii) cyclisation of the resulting material by treatment with triphenylphosphine in the presence of a base.

The saponification reaction in step (i) will generally be effected by treatment with a base. Suitable bases include inorganic hydroxides, e.g. an alkali metal hydroxide such as lithium hydroxide.

Suitable bases of use in step (iii) include organic amines, e.g. a trialkylamine such as triethylamine. The reaction is conveniently performed at ambient temperature in the presence of hexachloroethane and a suitable solvent, e.g. a cyclic ether such as tetrahydrofuran.

In another procedure, the compounds of formula (I) wherein A represents a group of formula (Ad) and Z represents a group of formula (Zw) or (Zx) as defined above, in which R^{1z} is other than hydrogen, may be prepared by a two-step procedure which comprises the following steps:
(i) reacting an alkali metal azide with a compound of formula (XVII):

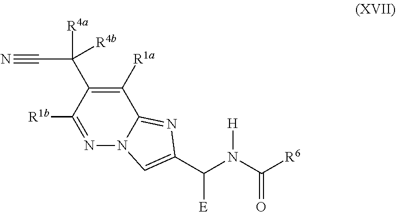

wherein E, R^{1a}, R^{1b}, R^{4a}, R^{4b} and R⁶ are as defined above; and
(ii) reacting the resulting material with a compound of formula R^{1z}-L³, wherein R^{1z} is as defined above (and is other than hydrogen), and L³ represents a suitable leaving group.

In step (i), the alkali metal azide is suitably sodium azide. The reaction is conveniently performed at an elevated temperature in the presence of ammonium chloride and a suitable solvent, e.g. a dipolar aprotic solvent such as N,N-dimethylformamide.

The leaving group L³ may suitably be a sulfonyloxy derivative, e.g. trifluoromethanesulfonyloxy.

Step (ii) will generally be accomplished in the presence of a base. Suitable bases include alkali metal carbonates, e.g. potassium carbonate. The reaction is conveniently effected at an elevated temperature in a suitable solvent, e.g. a carbonyl-containing solvent such as acetone.

The intermediates of formula (XVII) above may be prepared by a two-step procedure which comprises the following steps:
(i) reacting a compound of formula (XIII) as defined above with ammonia; and
(ii) reacting the material thereby obtained with trifluoroacetic anhydride in the presence of pyridine.

Step (i) is conveniently performed at an elevated temperature in a suitable solvent, e.g. a C₁₋₄ alkanol such as methanol.

Step (ii) is conveniently carried out at ambient temperature in a suitable solvent, e.g. a cyclic ether such as 1,4-dioxane.

The intermediates of formula (IV) above wherein A represents a group of formula (Ad) and Z represents a group of formula (Zq) as defined above, in which R^{2z} is hydrogen, may be prepared by reacting an azide derivative of formula R^{1z}—N₃ with a compound of formula (XVIII):

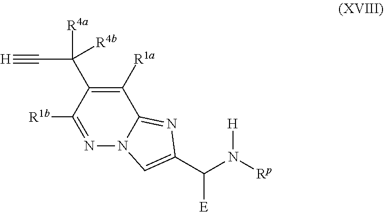

wherein E, R^{1a}, R^{1b}, R^{4a}, R^{4b}, R^{1z} and R^{p} are as defined above; in the presence of a transition metal catalyst.

Suitable transition metal catalysts of use in the above reaction include chloro-(pentamethylcyclopentadienyl)(cyclooctadiene)ruthenium(II).

The reaction is conveniently carried out at an elevated temperature in a suitable solvent or mixture of solvents. Typical solvents include alkyl ethers, e.g. tert-butyl methyl ether, or 1,2-dimethoxyethane; and cyclic ethers, e.g. tetrahydrofuran.

The intermediates of formula (XVIII) above may be prepared by reacting a compound of formula (XIX):

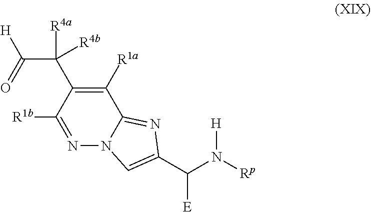

wherein E, R^{1a}, R^{1b}, R^{4a}, R^{4b} and R^{p} are as defined above; with dimethyl (1-diazo-2-oxopropyl)phosphonate.

The reaction is generally performed in the presence of a base. Suitably, the base may be an alkali metal carbonate, e.g. potassium carbonate. The reaction is conveniently effected at ambient temperature in a suitable solvent or mixture of solvents. Typical solvents include C₁₋₄ alkanols, e.g. methanol; and chlorinated solvents, e.g. dichloromethane.

The intermediates of formula (XIX) above may be prepared by a two-step procedure which comprises the following steps:
(i) removal of the 0-protecting group R^{s} from a compound of formula (XX):

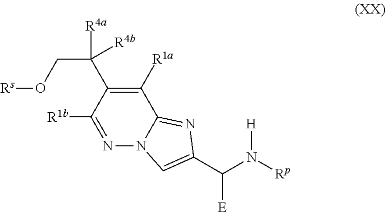

wherein E, R^{1a}, R^{1b}, R^{4a}, R^{4b} and R^{p} are as defined above, and R^{s} represents an 0-protecting group; and
(ii) treatment of the compound thereby obtained with an oxidising agent.

The O-protecting group R^{s} will suitably be acetyl.

Where R^{s} represents acetyl, the removal thereof in step (i) above may conveniently be effected by treatment with a base. Suitably, the base may be an alkali metal carbonate, e.g. potassium carbonate. The reaction is conveniently effected at ambient temperature in a suitable solvent, e.g. a C₁₋₄ alkanol such as methanol.

Suitable oxidising agents of use in step (ii) above include 1,1,1-tris(acetyloxy)-1,1-dihydro-1,2-benziodoxol-3-(1H)-one (Dess-Martin periodinane). The reaction is conveniently effected at ambient temperature in a suitable solvent, e.g. a chlorinated solvent such as dichloromethane.

Alternatively, the oxidising agent of use in step (ii) above may comprise sulfur trioxide pyridine complex, in which case the reaction may conveniently be accomplished in the presence of a base. Suitably, the base may be an organic amine, e.g. NN-diisopropylethylamine.

The intermediates of formula (XX) above may be prepared by reacting a compound of formula (IX) as defined above with a compound of formula (XXI):

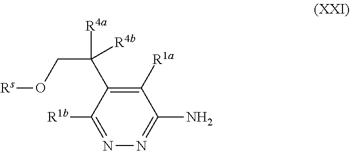

wherein R^{1a}, R^{1b}, R^{4a}, R^{4b} and R^{s} are as defined above; under conditions analogous to those described above for the reaction between compounds (VIII) and (IX).

The intermediates of formula (IV) above wherein A represents a group of formula (Ad) and Z represents a group of formula (Zw) as defined above may be prepared by reacting azidotrimethylsilane with a compound of formula (XXII):

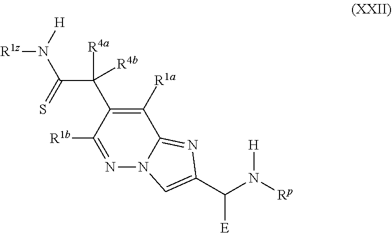

wherein E, R^{1a}, R^{1b}, R^{4a}, R^{4b}, R^{1z} and R^{p} are as defined above.

The reaction is generally accomplished in the presence of triphenylphosphine and an azodicarboxylate ester, e.g. diisopropyl azodicarboxylate (DIAD). The reaction is conveniently effected at ambient temperature in a suitable solvent, e.g. a cyclic ether such as tetrahydrofuran.

The intermediates of formula (XXII) above may be prepared by a three-step process which comprises:
(i) saponification of a compound of formula (VI) as defined above by treatment with a base;
(ii) reaction of the carboxylic acid derivative thereby obtained with a compound of formula R^{1z}—NH₂; under conditions analogous to those described above for the reaction between compound (III) and a carboxylic acid of formula R⁶—CO₂H; and
(iii) treatment of the carboxamide derivative thereby obtained with Lawesson's Reagent (2,4-bis(4-methoxyphenyl)-1,3-dithia-2,4-diphosphetane 2,4-disulfide).

The saponification reaction in step (i) will generally be effected by treatment with a base. Suitable bases include inorganic hydroxides, e.g. an alkali metal hydroxide such as lithium hydroxide.

Step (iii) is conveniently performed at an elevated temperature in a suitable solvent, e.g. a cyclic ether such as 1,4-dioxane.

In another procedure, the compounds of formula (I) above wherein A represents a group of formula (Ae), in which Y represents N—R⁷ and R⁷ represents —COR^{7a}, may be prepared by a process which comprises reacting a carboxylic acid of formula R^{7a}—CO₂H with a compound of formula (XXIII):

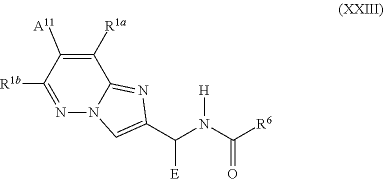

wherein
A¹¹ represents a group of formula (Ae-1):

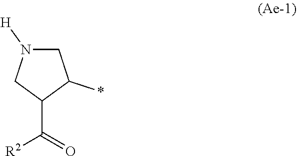

in which the asterisk (*) represents the point of attachment to the remainder of the molecule; and
E, R^{1a}, R^{1b}, R² and R⁶ are as defined above;
   under conditions analogous to those described above for the reaction between compound (III) and a carboxylic acid of formula R⁶—CO₂H.

Likewise, the intermediates of formula (IV) above wherein A represents a group of formula (Ae), in which Y represents N—R⁷ and R⁷ represents —COR^{7a}, may be prepared by reacting a carboxylic acid of formula R^{7a}—CO₂H with a compound of formula (XXIV):

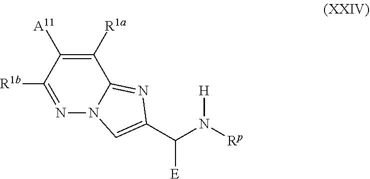

wherein E, A¹¹, R^{1a}, R^{1b} and R^{p} are as defined above; under conditions analogous to those described above for the reaction between compound (III) and a carboxylic acid of formula R⁶—CO₂H.

The compounds of formula (I) above wherein A represents a group of formula (Ae), in which Y represents N—R⁷ and R⁷ represents —CO₂R^{7a}, may be prepared by a process which comprises reacting a compound of formula (XXIII) as defined above with a compound of formula L^{4a}-CO₂R^{7a}, wherein L^{4a} represents a suitable leaving group, and R^{7a} is as defined above.

Likewise, the intermediates of formula (IV) above wherein A represents a group of formula (Ae), in which Y represents N—R⁷ and R⁷ represents —CO₂R^{7a}, may be prepared by reacting a compound of formula (XXIV) as defined above with a compound of formula L^{4a}-CO₂R^{7a}, wherein L^{4a} and R^{7a} are as defined above.

The leaving group L^{4a} is suitably a halogen atom, e.g. chloro. Alternatively, the leaving group L^{4a} may suitably be 2,5-dioxopyrrolidin-1-yloxy.

The reaction is conveniently accomplished in the presence of a base. Suitable bases include organic amines, e.g. a trialkylamine such as N,N-diisopropylethylamine or triethylamine. The reaction is conveniently performed at ambient temperature in a suitable solvent, e.g. a chlorinated solvent such as dichloromethane.

The compounds of formula (I) above wherein A represents a group of formula (Ae), in which Y represents N—R⁷ and R⁷ represents —SO₂R^{7b}, may be prepared by a process which comprises reacting a compound of formula (XXIII) as defined above with a compound of formula L^{4b}-SO₂R^{7b}, wherein L^{4b} represents a suitable leaving group, and R^{7b} is as defined above.

Likewise, the intermediates of formula (IV) above wherein A represents a group of formula (Ae), in which Y represents N—R⁷ and R⁷ represents —SO₂R^{7b}, may be prepared by reacting a compound of formula (XXIV) as defined above with a compound of formula L^{4b}-SO₂R^{7b}, wherein L^{4b} and R^{7b} are as defined above.

The leaving group L^{4b} is suitably a halogen atom, e.g. chloro.

The reaction is conveniently accomplished in the presence of a base. Suitable bases include organic amines, e.g. a trialkylamine such as N,N-diisopropylethylamine or triethylamine. The reaction is conveniently performed at ambient temperature in a suitable solvent, e.g. a chlorinated solvent such as dichloromethane.

The compounds of formula (I) above wherein A represents a group of formula (Ae), in which Y represents N—R⁷ and R⁷ represents C₁₋₆ alkyl, optionally substituted by one or more fluorine atoms, may be prepared by a process which comprises reacting a compound of formula (XXIII) as defined above with a compound of formula L⁵-R^{7c}, wherein L⁵ represents a suitable leaving group, and R^{7c} represents C₁₋₆ alkyl, optionally substituted by one or more fluorine atoms.

Likewise, the intermediates of formula (IV) above wherein A represents a group of formula (Ae), in which Y represents N—R⁷ and R⁷ represents C₁₋₆ alkyl, optionally substituted by one or more fluorine atoms, may be prepared by reacting a compound of formula (XXIV) as defined above with a compound of formula L⁵-R^{7c}, wherein L⁵ and R^{7c} are as defined above.

The leaving group L⁵ may suitably be a sulfonyloxy derivative, e.g. trifluoromethanesulfonyloxy.

The reaction is conveniently accomplished in the presence of a base. Suitable bases include organic amines, e.g. a trialkylamine such as triethylamine. The reaction is conveniently performed at ambient temperature in a suitable solvent, e.g. a chlorinated solvent such as dichloromethane.

The compounds of formula (I) above wherein A represents a group of formula (Ae), in which Y represents N—R⁷ and R⁷ represents C₃₋₉ cycloalkyl or C-linked C₃₋₇ heterocycloalkyl, optionally substituted by one or more fluorine atoms (e.g. 3,3-difluorocyclobutyl or oxetan-3-yl), may be prepared by a process which comprises reacting a compound of formula (XXIII) as defined above with the appropriate cycloalkanone or heterocycloalkanone, optionally substituted by one or more fluorine atoms (e.g. 3,3-difluorocyclobutanone or oxetan-3-one), in the presence of a reducing agent.

Likewise, the intermediates of formula (IV) above wherein A represents a group of formula (Ae), in which Y represents N—R⁷ and R⁷ represents C₃₋₉ cycloalkyl or C-linked C₃₋₇ heterocycloalkyl, optionally substituted by one or more fluorine atoms (e.g. 3,3-difluorocyclobutyl or oxetan-3-yl), may be prepared by reacting a compound of formula (XXIV) as defined above with the appropriate cycloalkanone or heterocycloalkanone, optionally substituted by one or more fluorine atoms (e.g. 3,3-difluorocyclobutanone or oxetan-3-one), in the presence of a reducing agent.

The reducing agent is suitably sodium triacetoxyborohydride. The reaction is conveniently performed in the presence of acetic acid.

As will be appreciated, the compounds of formula (XXIII) above correspond in their own right to compounds of formula (I) as defined above wherein A represents a group of formula (Ae), in which Y represents N—R⁷ and R⁷ represents hydrogen.

The compounds of formula (XXIII) above may conveniently be prepared by reacting the corresponding compound of formula (I) wherein A represents a group of formula (Ae) and Y represents N—R⁷, in which R⁷ represents —CO₂R^{7a} and R^{7a} represents tert-butyl, with an acid, e.g. a mineral acid such as hydrochloric acid, or an organic acid such as trifluoroacetic acid.

Likewise, the intermediates of formula (XXIV) above may conveniently be prepared by reacting the corresponding compound of formula (IV) wherein A represents a group of formula (Ae) and Y represents N—R⁷, in which R⁷ represents —CO₂R^{7a} and R^{7a} represents tert-butyl, with an acid, e.g. a mineral acid such as hydrochloric acid, or an organic acid such as trifluoroacetic acid.

The intermediates of formula (IV) above wherein A represents a group of formula (Ae) may be prepared by a three-step procedure which comprises the following steps:
(i) saponifying a compound of formula (XXV):

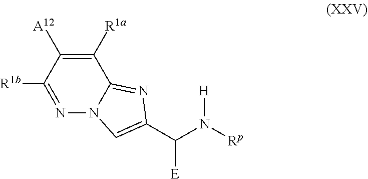

wherein
A¹² represents a group of formula (Ae-2):

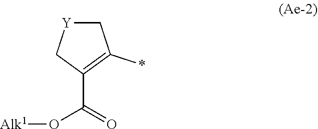

in which the asterisk (*) represents the point of attachment to the remainder of the molecule; and
E, Y, R^{1a}, R^{1b}, R^{p} and Alk¹ are as defined above;
(ii) reaction of the carboxylic acid derivative thereby obtained with a compound of formula R²—H; under conditions analogous to those described above for the reaction between compound (III) and a carboxylic acid of formula R⁶—CO₂H; and
(iii) reducing the C═C double bond in the Y-containing ring.

The saponification reaction in step (i) will generally be effected by treatment with a base. Suitable bases include inorganic hydroxides, e.g. an alkali metal hydroxide such as lithium hydroxide or sodium hydroxide. Step (i) is conveniently effected at ambient or elevated temperature in water and/or a suitable organic solvent, e.g. a cyclic ether such as tetrahydrofuran, or a C₁₋₄ alkanol such as methanol or ethanol.

Alternative coupling agents that may usefully be employed in step (ii) include 2-chloro-1-methylpyridinium iodide.

Reduction of the C═C double bond in step (iii) may conveniently be effected by catalytic hydrogenation, typically by treatment with hydrogen gas or ammonium formate in the presence of a hydrogenation catalyst, e.g. palladium on charcoal, or palladium hydroxide on charcoal.

The intermediates of formula (XXV) above may be prepared by reacting a compound of formula A¹²-L⁶ with a compound of formula (XXVI):

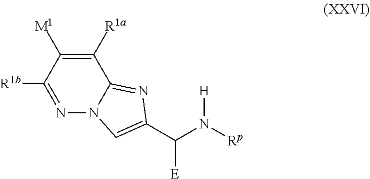

wherein M¹ represents —B(OH)₂ or a cyclic ester thereof formed with an organic diol, e.g. pinacol, 1,3-propanediol or neopentyl glycol, L⁶ represents a suitable leaving group, and E, A¹², R^{1a}, R^{1b} and R^{p} are as defined above; in the presence of a transition metal catalyst.

The leaving group L⁶ is suitably a halogen atom, e.g. chloro or bromo. Alternatively, the leaving group L⁶ may suitably be a sulfonyloxy derivative, e.g. methanesulfonyloxy or trifluoromethanesulfonyloxy.

The transition metal catalyst may suitably be tris(dibenzylideneacetone)-palladium(0), which may typically be employed in conjunction with 2-dicyclohexyl-phosphino-2′,4′,6′-triisopropylbiphenyl (XPhos). Typically, the reaction will be performed at an elevated temperature in the presence of potassium phosphate.

Alternatively, the transition metal catalyst may be [1,1′-bis(diphenylphosphino)-ferrocene]dichloropalladium(II). The reaction may conveniently be performed at an elevated temperature in the presence of potassium carbonate.

The intermediates of formula (XXV) above may alternatively be prepared by reacting a compound of formula A¹²-M¹ with a compound of formula (XIII) as defined above; in the presence of a transition metal catalyst; under conditions analogous to those described above for the reaction between compound (XXVI) and a compound of formula A¹²-L⁶.

By way of illustration, the intermediates of formula (XXVI) above wherein M¹ represents a cyclic ester of —B(OH)₂ formed with pinacol may be prepared by reacting bis(pinacolato)diboron with a compound of formula (XIII) as defined above; in the presence of a transition metal catalyst.

Likewise, the intermediates of formula A¹²-M¹ wherein M¹ represents a cyclic ester of —B(OH)₂ formed with pinacol may be prepared by reacting bis(pinacolato)diboron with a compound of formula A¹²-L⁶ as defined above; in the presence of a transition metal catalyst.

The transition metal catalyst may suitably be tris(dibenzylideneacetone)-palladium(0), which may typically be employed in conjunction with 2-dicyclohexyl-phosphino-2′,4′,6′-triisopropylbiphenyl (XPhos). Typically, the reaction will be performed at an elevated temperature in the presence of potassium acetate.

Where they are not commercially available, the starting materials of formula (VIII), (IX), (XIV), (XVI) and (XXI) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art. By way of example, a compound comprising a N-BOC moiety (wherein BOC is an abbreviation for tert-butoxycarbonyl) may be converted into the corresponding compound comprising a N—H moiety by treatment with an acid, e.g. a mineral acid such as hydrochloric acid, or an organic acid such as trifluoroacetic acid.

A compound comprising a N—H functionality may be alkylated, e.g. methylated, by treatment with a suitable alkyl halide, e.g. iodomethane, typically in the presence of a base, e.g. an inorganic carbonate such as sodium carbonate.

A compound comprising a N—H functionality may be acylated, e.g. acetylated, by treatment with a suitable acyl halide, e.g. acetyl chloride, typically in the presence of a base, e.g. an organic base such as N,N-diisopropylethylamine or triethylamine. Similarly, a compound comprising a N—H functionality may be acylated, e.g. acetylated, by treatment with a suitable acyl anhydride, e.g. acetic anhydride, typically in the presence of a base, e.g. an organic base such as triethylamine.

Simlarly, a compound comprising a N—H functionality may be converted into the corresponding compound comprising a N—S(O)₂Alk¹ functionality (wherein Alk¹ is as defined above) by treatment with the appropriate C₁₋₄ alkylsulfonyl chloride reagent, e.g. methylsulfonyl chloride, typically in the presence of a base, e.g. an organic base such as triethylamine.

Simlarly, a compound comprising a N—H functionality may be converted into the corresponding compound comprising a carbamate or urea moiety respectively by treatment with the appropriate chloroformate or carbamoyl chloride reagent, typically in the presence of a base, e.g. an organic base such as triethylamine or N,N-diisopropylethylamine. Alternatively, a compound comprising a N—H functionality may be converted into the corresponding compound comprising a urea moiety by treatment with the appropriate amine-substituted (3-methylimidazol-3-ium-1-yl)methanone iodide derivative, typically in the presence of a base, e.g. an organic base such as triethylamine. Alternatively, a compound comprising a N—H functionality may be converted into the corresponding compound comprising a urea moiety N—C(O)N(H)Alk¹ (wherein Alk¹ is as defined above) by treatment with the appropriate isocyanate derivative Alk¹-N═C═O, typically in the presence of a base, e.g. an organic base such as triethylamine.

A compound comprising a N—H functionality may be converted into the corresponding compound comprising a N—C(H) functionality by treatment with the appropriate aldehyde or ketone in the presence of a reducing agent such as sodium triacetoxyborohydride.

A compound comprising a C₁₋₄ alkoxycarbonyl moiety —CO₂Alk¹ (wherein Alk¹ is as defined above) may be converted into the corresponding compound comprising a carboxylic acid (—CO₂H) moiety by treatment with a base, e.g. an alkali metal hydroxide salt such as lithium hydroxide. Alternatively, a compound comprising a tert-butoxycarbonyl moiety may be converted into the corresponding compound comprising a carboxylic acid (—CO₂H) moiety by treatment with trifluoroacetic acid.

A compound comprising a carboxylic acid (—CO₂H) moiety may be converted into the corresponding compound comprising an amide moiety by treatment with the appropriate amine, under conditions analogous to those described above for the reaction between compound (III) and a carboxylic acid of formula R⁶—CO₂H.

A compound comprising a C₁₋₄ alkoxycarbonyl moiety —CO₂Alk¹ (wherein Alk¹ is as defined above) may be converted into the corresponding compound comprising a hydroxymethyl (—CH₂OH) moiety by treatment with a reducing agent such as lithium aluminium hydride.

A compound comprising a C₁₋₄ alkylcarbonyloxy moiety —OC(O)Alk¹ (wherein Alk¹ is as defined above), e.g. acetoxy, may be converted into the corresponding compound comprising a hydroxy (—OH) moiety by treatment with a base, e.g. an alkali metal hydroxide salt such as sodium hydroxide.

A compound comprising a halogen atom, e.g. bromo, may be converted into the corresponding compound comprising an optionally substituted aryl, heterocycloalkenyl or heteroaryl moiety by treatment with the appropriately substituted aryl, heterocycloalkenyl or heteroaryl boronic acid or a cyclic ester thereof formed with an organic diol, e.g. pinacol, 1,3-propanediol or neopentyl glycol. The reaction is typically effected in the presence of a transition metal catalyst, and a base. The transition metal catalyst may be [1,1′-bis(diphenylphosphino)ferrocene]dichloropalladium(II). In the alternative, the transition metal catalyst may be tris(dibenzylideneacetone)dipalladium(0), which may advantageously be employed in conjunction with 2-dicyclohexylphosphino-2′,4′,6′-triisopropylbiphenyl (XPhos). Suitably, the base may be an inorganic base such as sodium carbonate or potassium carbonate.

A compound comprising a halogen atom, e.g. bromo, may be converted into the corresponding compound comprising an optionally substituted aryl or heteroaryl moiety via a two-step procedure which comprises: (i) reaction with bis(pinacolato)diboron; and (ii) reaction of the compound thereby obtained with an appropriately substituted bromoaryl or bromoheteroaryl derivative. Step (i) is conveniently effected in the presence of a transition metal catalyst such as [1,1′-bis(diphenylphosphino)ferrocene]-dichloropalladium(II), and potassium acetate. Step (ii) is conveniently effected in the presence of a transition metal catalyst such as [1,1′-bis(diphenylphosphino)ferrocene]-dichloropalladium(II), and a base, e.g. an inorganic base such as sodium carbonate or potassium carbonate.

A compound comprising a cyano (—CN) moiety may be converted into the corresponding compound comprising a 1-aminoethyl moiety by a two-step process which comprises: (i) reaction with methylmagnesium chloride, ideally in the presence of titanium(IV) isopropoxide; and (ii) treatment of the resulting material with a reducing agent such as sodium borohydride. If an excess of methylmagnesium chloride is employed in step (i), the corresponding compound comprising a 1-amino-1-methylethyl moiety may be obtained.

A compound comprising the moiety —S— may be converted into the corresponding compound comprising the moiety —S(O)(NH)— by treatment with (diacetoxyiodo)benzene and ammonium carbamate.

A compound comprising a C═C double bond may be converted into the corresponding compound comprising a CH—CH single bond by treatment with gaseous hydrogen in the presence of a hydrogenation catalyst, e.g. palladium on charcoal.

A compound comprising an aromatic nitrogen atom may be converted into the corresponding compound comprising an N-oxide moiety by treatment with a suitable oxidising agent, e.g. 3-chloroperbenzoic acid.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt.

In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode.

Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Greene's Protective Groups in Organic Synthesis, ed. P. G. M. Wuts, John Wiley & Sons, 5^{th} edition, 2014. The protecting groups may be removed at any convenient subsequent stage utilising methods known from the art.

The compounds in accordance with this invention potently inhibit IL-17 induced IL-6 release from human dermal fibroblasts. Thus, when tested in the HDF cell line assay described below, compounds of the present invention exhibit a pIC₅₀ value of 5.0 or more, generally of 6.0 or more, usually of 7.0 or more, typically of 7.2 or more, suitably of 7.5 or more, ideally of 7.8 or more, and preferably of 8.0 or more (pIC₅₀ equals −log₁₀[IC₅₀], in which IC₅₀ is expressed as a molar concentration, so the skilled person will appreciate that a higher pIC₅₀ figure denotes a more active compound).

Inhibition of IL-17A Induced IL-6 Release from Dermal Fibroblast Cell Line

The purpose of this assay is to test the neutralising ability to IL-17 proteins, in a human primary cell system. Stimulation of normal human dermal fibroblasts (HDF) with IL-17 alone produces only a very weak signal but in combination with certain other cytokines, such as TNFα, a synergistic effect can be seen in the production of inflammatory cytokines, i.e. IL-6.

HDFs were stimulated with IL-17A (50 pM) in combination with TNF-α (25 pM). The resultant IL-6 response was then measured using a homogenous time-resolved FRET kit from Cisbio. The kit utilises two monoclonal antibodies, one labelled with Eu-Cryptate (Donor) and the second with d2 or XL665 (Acceptor). The intensity of the signal is proportional to the concentration of IL-6 present in the sample (Ratio is calculated by 665/620×104).

The ability of a compound to inhibit IL-17 induced IL-6 release from human dermal fibroblasts is measured in this assay.

HDF cells (Sigma #106-05n) were cultured in complete media (DMEM+10% FCS+2 mM L-glutamine) and maintained in a tissue culture flask using standard techniques. Cells were harvested from the tissue culture flask on the morning of the assay using TrypLE (Invitrogen #12605036). The TrypLE was neutralised using complete medium (45 mL) and the cells were centrifuged at 300×g for 3 minutes. The cells were re-suspended in complete media (5 mL) counted and adjusted to a concentration of 3.125×10⁴ cells/mL before being added to the 384 well assay plate (Corning #3701) at 40 μL per well. The cells were left for a minimum of three hours, at 37° C./5% CO₂, to adhere to the plate.

Compounds were serially diluted in DMSO before receiving an aqueous dilution into a 384 well dilution plate (Greiner #781281), where 5 μL from the titration plate was transferred to 45 μL of complete media and mixed to give a solution containing 10% DMSO.

Mixtures of TNFα and IL-17 cytokine were prepared in complete media to final concentrations of TNFα 25 pM/IL-17A 50 pM, then 30 μL of the solution was added to a 384 well reagent plate (Greiner #781281).

L from the aqueous dilution plate was transferred to the reagent plate containing 30 μL of the diluted cytokines, to give a 2.5% DMSO solution. The compounds were incubated with the cytokine mixtures for 5 h at 37° C. After the incubation, 10 μL was transferred to the assay plate, to give a 0.5% DMSO solution, then incubated for 18-20 h at 37° C./5% CO₂.

From the Cisbio IL-6 FRET kit (Cisbio #62IL6PEB) europium cryptate and Alexa 665 were diluted in reconstitution buffer and mixed 1:1, as per kit insert. To a white low volume 384 well plate (Greiner #784075) were added FRET reagents (10 μL), then supernatant (10 μL) was transferred from the assay plate to Greiner reagent plate. The mixture was incubated at room temperature for 3 h with gentle shaking (<400 rpm) before being read on a Synergy Neo 2 plate reader (Excitation: 330 nm; Emission: 615/645 nm).

When tested in the HDF cell line assay as described above, the compounds of the accompanying Examples were found to exhibit the following pIC₅₀ values.

| Example | pIC50 |
|---|---|
| 1 | 7.4 |
| 2 | 8.0 |
| 3A/Peak 1 | 7.1 |
| 3B/Peak 2 | 6.1 |
| 4A/Peak 1 | 8.4 |
| 4B/Peak 2 | 6.8 |
| 5 | 7.7 |
| 6A/Peak 1 | 8.1 |
| 6B/Peak 2 | 6.6 |
| 7 | 7.6 |
| 8 | 6.8 |
| 9 | 7.2 |
| 10A/Peak 1 | 7.3 |
| 10B/Peak 2 | 6.6 |
| 11 | 6.5 |
| 12 | 7.5 |
| 13 | 7.6 |
| 14 | 7.2 |
| 15 | 7.7 |
| 16 | 7.6 |
| 17 | 8.2 |

The following Examples illustrate the preparation of compounds according to the invention.

### EXAMPLES

### Abbreviations

| | |
|---|---|
| DCM: dichloromethane | THF: tetrahydrofuran |
| MeOH: methanol | EtOH: ethanol |
| DMSO: dimethyl sulfoxide | DIPEA: N,N-diisopropylethylamine |
| DMF: N,N-dimethylformamide | DMA: N,N-dimethylacetamide |
| EtOAc: ethyl acetate | TFA: trifluoroacetic acid |
| IPA: isopropyl alcohol | DMAP: 4-(dimethylamino)pyridine |
| TBME: tert-butyl methyl ether | LDA: lithium diisopropylamide |
| NBS: N-bromosuccinimide | DIAD: diisopropyl azodicarboxylate |
| Xantphos: 4,5-bis(diphenylphosphino)-9,9- | |
| dimethylxanthene | |
| T3P ®: propylphosphonic anhydride solution | |
| HATU: 1-[bis(dimethylamino)methylene]-1H- | |
| 1,2,3-triazolo[4,5-b]pyridinium 3-oxid | |
| hexafluorophosphate | |
| {Ir[dF(CF₃)ppy]₂(dtbpy)}PF₆: [4,4′-bis(1,1- | |
| dimethylethyl)-2,2′-bipyridine-N1,N1′]bis- | |
| {3,5-difluoro-2-[5-(trifluoromethyl)-2-pyridinyl- | |
| N]phenyl-C}iridium(III) hexafluoro-phosphate | |
| Lawesson's Reagent: 2,4-bis(4-methoxyphenyl)- | |
| 1,3-dithia-2,4-diphosphetane 2,4-disulfide | |
| h: hour | r.t.: room temperature |
| M: mass; molar | RT: retention time |
| HPLC: High Performance Liquid Chromatography | |
| LCMS: Liquid Chromatography Mass Spectrometry | |
| SFC: Supercritical Fluid Chromatography | |
| FCC: Flash Column Chromatography | |

### Analytical and Separation Methods

### Method 1

X-Bridge C18 Waters 2.1×20 mm, 2.5 μm column

### Column Temperature: 40° C.

Mobile Phase A: 10 mM ammonium formate in water+0.1% ammonia solution
Mobile Phase B: acetonitrile+5% water+0.1% ammonia solution
Gradient program: Flow rate 1 mL/minute

| Time | A % | B % |
|---|---|---|
| 0.00 | 95.00 | 5.00 |
| 4.00 | 5.00 | 95.00 |
| 5.00 | 5.00 | 95.00 |
| 5.10 | 95.00 | 5.00 |

### Method 2

Waters UPLC® BEH™ C18, Part No. 186002352, 2.1×100 mm, 1.7 μm column

### Column Temperature: 40° C.

Mobile Phase A: 2 mM ammonium bicarbonate, buffered to pH 10
Mobile Phase B: acetonitrile
Gradient program Flow rate 0.6 mL/minute

| Time | A % | B % |
|---|---|---|
| 0.00 | 95.00 | 5.00 |
| 5.30 | 0 | 100 |
| 5.80 | 0 | 100 |
| 5.82 | 95.00 | 5.00 |
| 7.00 | 95.00 | 5.00 |

### Method 3

Phenomenex, Kinetex-XB C18, 2.1 mm×100 mm, 1.7 μm column

### Column Temperature: 40° C.

Mobile Phase A: 0.1% formic acid in water
Mobile Phase B: 0.1% formic acid in acetonitrile
Gradient program: Flow rate 0.6 mL/minute; Injection volume 1 μL

| Time | A % | B % |
|---|---|---|
| 0.00 | 95 | 5 |
| 5.30 | 0 | 100 |
| 5.80 | 0 | 100 |
| 5.82 | 95 | 5 |
| 7.00 | 95 | 5 |

UV 215 nm, PDA spectrum 200-400 nm, step 1 nm

### MSD Scan Positive 150-850

### Method 4

X-Bridge C18 Waters 2.1×20 mm, 2.5 μm column
Mobile Phase A: 10 mM ammonium formate in water+0.1% ammonia solution
Mobile Phase B: acetonitrile+5% water+0.1% ammonia solution
Gradient program: Flow rate 1 mL/minute

| Time | A % | B % |
|---|---|---|
| 0.00 | 95.00 | 5.00 |
| 1.50 | 5.00 | 95.00 |
| 2.25 | 5.00 | 95.00 |
| 2.50 | 95.00 | 5.00 |

### Method 5

X-Bridge C18 Waters 2.1×20 mm, 2.5 μm column

### Column Temperature: 40° C.

Mobile Phase A: 10 mM ammonium formate in water+0.1% formic acid
Mobile Phase B: acetonitrile+5% water+0.1% formic acid
Gradient program: Flow rate 1 mL/minute

| Time | A % | B % |
|---|---|---|
| 0.00 | 95.00 | 5.00 |
| 1.50 | 5.00 | 95.00 |
| 2.25 | 5.00 | 95.00 |
| 2.50 | 95.00 | 5.00 |

### Method 6

X-Bridge C18 Waters 2.1×20 mm, 2.5 μm column

### Column Temperature: 40° C.

Mobile Phase A: 10 mM ammonium formate in water+0.1% formic acid
Mobile Phase B: acetonitrile+5% water+0.1% formic acid
Gradient program: Flow rate 1 mL/minute

| Time | A % | B % |
|---|---|---|
| 0.00 | 95.00 | 5.00 |
| 4.00 | 5.00 | 95.00 |
| 5.00 | 5.00 | 95.00 |
| 5.10 | 95.00 | 5.00 |

### Method 7

Phenomenex Gemini NX-C18 2×20 mm, 3 μm column
Mobile Phase A: 10 mM ammonium formate in water+0.1% ammonia solution
Mobile Phase B: acetonitrile+5% water+0.1% ammonia solution
Gradient program: Flow rate 1 mL/minute

| Time | A % | B % |
|---|---|---|
| 0.00 | 95.00 | 5.00 |
| 4.00 | 5.00 | 95.00 |
| 5.00 | 5.00 | 95.00 |
| 5.10 | 95.00 | 5.00 |

### Method 8

Waters Acquity UPLC BEH C18 2.1×50 mm, 1.7 μm column
Mobile Phase A: 10 mM ammonium formate in water+0.1% ammonia solution
Mobile Phase B: acetonitrile+5% water+0.1% ammonia solution
Gradient program: Flow rate 1.5 mL/minute

| Time | A % | B % |
|---|---|---|
| 0.00 | 95.00 | 5.00 |
| 0.10 | 95.00 | 5.00 |
| 3.50 | 5.00 | 95.00 |
| 4.00 | 5.00 | 95.00 |
| 4.05 | 95.00 | 5.00 |

### Method 9

Waters Sunfire C18 30×100 mm, 10 μm column
Column Temperature: room temperature
Mobile Phase A: water+0.1% formic acid
Mobile Phase B: acetonitrile+0.1% formic acid
Gradient program: Flow rate 40 mL/minute

| Time | A % | B % |
|---|---|---|
| 0.00 | 70 | 30 |
| 0.55 | 70 | 30 |
| 11.0 | 5 | 95 |
| 13.1 | 5 | 95 |
| 13.31 | 70 | 30 |

### Method 10

Waters Acquity UPLC BEH C18 2.1×50 mm, 1.7 μm column
Mobile Phase A: 10 mM ammonium formate in water+0.1% formic acid
Mobile Phase B: acetonitrile+5% water+0.1% formic acid
Gradient program: Flow rate 1.5 mL/minute

| Time | A % | B % |
|---|---|---|
| 0.00 | 95.00 | 5.00 |
| 0.10 | 95.00 | 5.00 |
| 3.50 | 5.00 | 95.00 |
| 4.00 | 5.00 | 95.00 |
| 4.05 | 95.00 | 5.00 |

### Method 11

Phenomenex Gemini NX-C18 2×20 mm, 3 μm column
Mobile Phase A: 10 mM ammonium formate in water+0.1% ammonia solution
Mobile Phase B: acetonitrile+5% water+0.1% ammonia solution
Gradient program: Flow rate 1 mL/minute

| Time | A % | B % |
|---|---|---|
| 0.00 | 95.00 | 5.00 |
| 1.50 | 5.00 | 95.00 |
| 2.25 | 5.00 | 95.00 |
| 2.50 | 95.00 | 5.00 |

### Intermediate 1

### tert-Butyl N-(6-chloro-5-methylpyridazin-3-yl)carbamate

To a stirred solution of 6-chloro-5-methylpyridazin-3-amine (4.21 g, 29.3 mmol) and DIPEA (20 mL, 0.117 mol) in DMF (45 mL) were added di-tert-butyl dicarbonate (14.55 g, 66.7 mmol) and DMAP (358 mg, 2.93 mmol) sequentially. The reaction mixture was stirred at r.t. for 3 h, then concentrated to dryness under high vacuum. The resulting brown solid was dissolved in methanol (45 mL), and dipotassium carbonate (4.05 g, 29.3 mmol) was added. The reaction mixture was stirred at r.t. for 16 h, then concentrated under vacuum, redissolved in EtOAc (250 mL) and washed with water (200 mL). The resulting suspension was filtered through a pad of Celite®, washing with EtOAc (500 mL). The layers were separated, and the aqueous layer was re-extracted with EtOAc (2×50 mL). The combined organic phases were washed with brine (40 mL), then dried over sodium sulfate and concentrated under vacuum. The resulting brown solid was purified by silica column chromatography, eluting with 0-50% EtOAc in heptane, to afford the title compound (6.87 g, 79%) as a white solid. LCMS (Method 4): [M+H]⁺ m/z 244.0, RT 1.79 minutes.

### Intermediate 2

### 2-[6-(tert-Butoxvcarbonylamino)-3-chloropyridazin-4-yl]acetic acid

To a solution of Intermediate 1 (5.02 g, 20.6 mmol) in THE (60 mL) at −50° C. was added 2M LDA in THF/n-heptane/ethylbenzene (31 mL, 61.8 mmol) dropwise. The reaction mixture was stirred at −50° C. for 5 minutes, then allowed to warm slowly to −30° C. and stirred for 20 minutes. Gaseous CO₂ was bubbled into the reaction mixture (the internal temperature reached −10° C. on addition). The reaction mixture was re-cooled to −50° C. and stirred for a further 40 minutes, then diluted with saturated aqueous NH₄Cl solution (20 mL) and EtOAc (20 mL). The organic layer was discarded. The pH of the aqueous layer was adjusted to pH 3-4 using 2M aqueous HCl solution. The resulting aqueous layer was extracted with ethyl acetate (3×100 mL). The combined organic layers were dried over sodium sulfate and concentrated under vacuum to afford the title compound (4.7 g, 62%) as an orange oil. δ_{H} (500 MHz, DMSO-d₆) 10.66 (s, 1H), 8.21 (s, 1H), 3.83 (s, 2H), 1.50-1.47 (m, 9H). LCMS (Method 5): [M-^{t}Bu]⁺ m/z 232.0, RT 1.55 minutes.

### Intermediate 3

### Methyl 2-[6-(tert-butoxycarbonylamino)-3-chloropyridazin-4-yl]acetate

To a solution of Intermediate 2 (79%, 4.70 g, 12.9 mmol) in methanol (40 mL) was added 4M HCl in 1,4-dioxane (10 mL, 40.0 mmol). The reaction mixture was stirred for 4 h. The pH of the reaction was adjusted to pH 7 using saturated aqueous NaHCO₃ solution, then water (100 mL) was added. The resulting solid precipitate was collected by vacuum filtration and washed with minimal water, then dried in a vacuum oven at 40° C. for 16 h, to afford the title compound (3.83 g, 93%) as a white solid. LCMS (Method 3): [M+H]⁺ m/z 302.2, RT 2.84 minutes.

### Intermediate 4

### Methyl 2-[6-(tert-butoxycarbonylamino)-3-chloropyridazin-4-yl]-4,4-difluorobut-2-enoate

To a solution of Intermediate 3 (1.22 g, 4.04 mmol) in 1,4-dioxane (24 mL) were added triethylamine (1127 μL, 8.09 mmol) and 1-ethoxy-2,2-difluoroethanol (637 mg, 5.05 mmol). The reaction mixture was heated at 102° C. for 3 h. Additional portions of triethylamine (0.56 mL, 4.04 mmol) and 1-ethoxy-2,2-difluoroethanol (0.56 mL, 5.05 mmol) were added. The reaction mixture was heated at 102° C. for a further 3 h, then concentrated under vacuum. The resulting brown oil was purified by silica column chromatography, eluting with 0-40% EtOAc in heptane, to afford the title compound (965 mg, 66%) as a white solid. LCMS (Method 5): [M+H]⁺ m/z 364.0, RT 1.92 minutes.

### Intermediate 5

### Methyl 2-(6-amino-3-chloropyridazin-4-yl)-4,4-difluorobut-2-enoate

Intermediate 4 (90%, 528 mg, 1.31 mmol) was stirred in DCM (2.5 mL) and TFA (1.5 mL, 19.6 mmol) for 2 h at r.t. The reaction mixture was diluted with DCM (30 mL), washed with saturated aqueous NaHCO₃ solution (2×10 mL) and brine (10 mL), then dried over sodium sulfate and concentrated under vacuum, to afford the title compound (90% purity) (357 mg, 93%) as a yellow oil. LCMS (Method 5): [M+H]⁺ m/z 264.0, RT 1.44 minutes.

### Intermediate 6

### Methyl 2-{2-[(S)-benzyloxycarbonylamino(4,4-difluorocyclohexyl)methyl]-6-chloroimidazo[1,2-b]pyridazin-7-yl}-4,4-difluorobut-2-enoate

Intermediate 5 (90%, 357 mg, 1.22 mmol) and benzyl N-[(1S)-3-bromo-1-(4,4-difluorocyclohexyl)-2-oxopropyl]carbamate (640 mg, 1.58 mmol) were stirred in dry 1,4-dioxane (5 mL), and 2,6-dimethylpyridine (369 μL, 3.17 mmol) was added. The reaction mixture was sealed and heated at 80° C. for 3.5 h. The residue was diluted with ethyl acetate (50 mL), washed with water (20 mL), saturated aqueous NH₄Cl solution (20 mL), water (20 mL) and brine (20 mL), then dried over sodium sulfate and concentrated under vacuum. The material was purified by silica column chromatography, eluting with a gradient of 0-100% ethyl acetate in heptane, to afford the title compound (90% purity) (673 mg, 87%) as a yellow/orange foam. LCMS (Method 5): [M+H]⁺ m/z 569.2, RT 2.09 minutes.

### Intermediate 7

### Methyl 2-{2-[(S)-amino(4,4-difluorocyclohexyl)methyl]imidazo[1,2-b]pyridazin-7-yl}-4,4-difluorobutanoate

Intermediate 6 (90%, 600 mg, 0.949 mmol) was dissolved in methanol (20 mL) and 1M aqueous HCl (5.0 mL, 5.00 mmol) was added, followed by 10% Pd/C (50% wet) (5.0%, 202 mg, 0.0949 mmol). The reaction mixture was stirred under 1 atmosphere of H₂ for 2.5 h, then filtered through Celite® and concentrated under vacuum to remove the majority of the methanol. The residue was diluted with ethyl acetate (50 mL) and washed with saturated aqueous NaHCO₃ solution (30 mL). The aqueous layer was extracted further with ethyl acetate (20 mL). The organic extracts were combined and washed with brine (20 mL), then dried over sodium sulfate and concentrated under vacuum. The residue was purified by silica column chromatography, eluting with a gradient of 0-90% MeOH in DCM, to afford the title compound (95% purity) (260 mg, 65%) as an orange oil. LCMS (Method 6): [M+H]⁺ m/z 403.2, RT 1.98 minutes.

### Intermediate 8

### Methyl 2-{2-[(S)-(tert-butoxycarbonylamino)(4,4-difluorocyclohexyl)methyl]imidazo-[1,2-b]pyridazin-7-yl}-4,4-difluorobutanoate

Intermediate 7 (95%, 500 mg, 1.18 mmol) was dissolved in DCM (20 mL) and di-tert-butyl dicarbonate (309 mg, 1.42 mmol) was added. The reaction mixture was stirred at r.t. for 2.5 h, then diluted with DCM (20 mL), washed with water (2×20 mL), dried over sodium sulfate and concentrated under vacuum, to afford the title compound (90% purity) (660 mg, 100%) as a brown foam. LCMS (Method 5): [M+H]⁺ m/z 503.2, RT 1.89 minutes.

### Intermediate 9

### 2-{2-[(S-(tert-Butoxycarbonylamino)(4,4-difluorocyclohexyl)methyl]imidazo[1,2-b]-pyridazin-7-yl}-4,4-difluorobutanoic acid

Intermediate 8 (90%, 660 mg, 1.18 mmol) was dissolved in water (5 mL), methanol (5 mL) and THE (5 mL). Lithium hydroxide hydrate (1:1:1) (198 mg, 4.73 mmol) was added, and the reaction mixture was stirred for 45 minutes, then concentrated under vacuum to remove the majority of the organic solvent. The remaining mixture was diluted with ethyl acetate (30 mL) and water (30 mL), then the aqueous portion was adjusted to pH 5 using 1M aqueous HCl (5 mL). The mixture was separated and the aqueous layer was extracted with further ethyl acetate (30 mL). The combined organic extracts were dried over sodium sulfate, and concentrated under vacuum, to afford the title compound (95% purity) (610 mg, 100%) as a yellow foam. LCMS (Method 5): [M+H]⁺ m/z 489.2, RT 1.85 minutes.

### Intermediate 10

### tert-Butyl N-[(S)-(4,4-difluorocyclohexyl){7-[3,3-difluoro-1-(2,2,2-trifluoroethyl-carbamoyl)propyl]imidazo[1,2-b]pyridazin-2-yl}methyl]carbamate

Intermediate 9 (95%, 610 mg, 1.19 mmol) was dissolved in DMF (8 mL). 2,2,2-Trifluoroethanamine (0.10 mL, 1.30 mmol) and DIPEA (0.41 mL, 2.37 mmol) were added, followed by HATU (541 mg, 1.42 mmol). The reaction mixture was stirred at r.t. for 70 minutes, then further 2,2,2-trifluoroethanamine (0.10 mL, 1.30 mmol) and HATU (150 mg, 0.39 mmol) were added. The reaction mixture was stirred for 20 minutes, then diluted with water (20 mL) and sonicated. The mixture was filtered, and the sticky solid was washed with a small amount of water. The solid was dissolved in ethyl acetate, then dried over sodium sulfate and concentrated under vacuum, to afford the title compound (90% purity) (753 mg, 100%) as an orange foam. LCMS (Method 5): [M+H]⁺ m/z 570.2, RT 1.98 minutes.

### Intermediate 11

### 2-{2-[(S)-Amino(4,4-difluorocyclohexyl)methyl]imidazo[1,2-b]pyridazin-7-yl}-4,4-difluoro-N-(2,2,2-trifluoroethyl)butanamide

Intermediate 10 (90%, 753 mg, 1.19 mmol) was stirred in DCM (7.5 mL) and TFA (2.5 mL) for 45 minutes, then concentrated under vacuum. The residue was dissolved in DCM (50 mL) and washed with saturated aqueous NaHCO₃ solution (2×20 mL) and brine (20 mL), then dried over sodium sulfate and concentrated under vacuum. The material was purified by silica column chromatography, eluting with a gradient of 0-10% MeOH in DCM, to afford the title compound (364 mg, 65%) as a pale brown solid. LCMS (Method 6): [M+H]⁺ m/z 470.2, RT 2.02 minutes.

### Intermediate 12

### Ethyl 2-(2-{(S)-(4,4-difluorocyclohexyl)[(4-methyl-1,2,5-oxadiazole-3-carbonyl)amino]-methyl}imidazo[1,2-b]pyridazin-7-yl)-4,4-difluorobutanoate

HATU (161 mg, 0.423 mmol) was added to a mixture of 4-methyl-1,2,5-oxadiazole-3-carboxylic acid (54 mg, 0.423 mmol) and DIPEA (123 μL, 0.705 mmol) in DMF (3 mL) at r.t. The reaction mixture was stirred at r.t. for 5 minutes, then a solution of Intermediate 21 (128 mg, 0.282 mmol) and DIPEA (123 μL, 0.705 mmol) in DMF (3 mL) was added. The reaction mixture was stirred at r.t. for 16 h, then diluted with EtOAc (50 mL) and washed with water (3×60 mL). The organic phase was dried over sodium sulfate and concentrated to dryness under vacuum. The crude residue was purified by silica column chromatography, eluting with 10-60% EtOAc in heptane, to afford the title compound (112 mg, 55%) as a tan gum. LCMS (Method 5): [M+H]⁺ m/z 527, RT 1.99 minutes.

### Intermediate 13

### 2-(2-{(S)-(4,4-Difluorocyclohexyl)[(4-methyl-1,2,5-oxadiazole-3-carbonyl)amino]-methyl}imidazo[1,2-b]pyridazin-7-yl)-4,4-difluorobutanoic acid

A mixture of lithium hydroxide monohydrate (20 mg, 0.468 mmol) and Intermediate 12 (112 mg, 0.213 mmol) in THE (2.5 mL), water (0.4 mL) and methanol (1 mL) was stirred at r.t. for 16 h. The reaction mixture was concentrated to low volume under vacuum and diluted with EtOAc (50 mL), then 0.5M aqueous HCl was added until pH 3 was achieved. The layers were separated, and the aqueous layer was extracted with EtOAc (2×30 mL). The organic phase was washed with brine and dried over sodium sulfate, then concentrated to dryness under vacuum to afford the title compound (90 mg, 82%) as a yellow solid. LCMS (Method 5): [M+H]⁺ m/z 500, RT 1.82 minutes.

### Intermediate 14

### tert-Butyl N-[(1R)-2-methyl-1-(methylcarbamoyl)propyl]carbamate

To a solution of N-(tert-butoxycarbonyl)-D-valine (1200 mg, 5.52 mmol) in DCM (25 mL) were added DIPEA (2.2 mL, 12.2 mmol) and (1H-benzotriazol-1-yloxy)[tris-(dimethylamino)]phosphonium hexafluorophosphate (2443 mg, 5.52 mmol). Methyl-amine (2M, 4.4 mL, 8.84 mmol) was added, and the solution was stirred for 18 h. DCM (25 mL) was added, and the reaction mixture was washed with saturated aqueous NH₄Cl solution (10 mL) and saturated aqueous NaHCO₃ solution (10 mL), then dried over magnesium sulfate. The solvent was removed. Purification of the resulting solid by silica column chromatography, eluting with 0-50% EtOAc in heptane, afforded the title compound (1.32 g, 98%) as a white solid. δ_{H} (400 MHz, DMSO-d₆) 7.76 (s, 1H), 6.58 (d, J 8.5 Hz, 1H), 3.67-3.76 (m, 1H), 2.58 (d, J 4.6 Hz, 3H), 1.83-1.92 (m, 1H), 1.38 (s, 9H), 0.81 (dd, J 6.7, 2.9 Hz, 6H). LCMS (Method 5): did not ionise, RT 1.47 minutes.

### Intermediate 15

### (2R)-2-Amino-N,3-dimethylbutanamide trifluoroacetic acid salt

To a solution of Intermediate 14 (300 mg, 1.30 mmol) in DCM (3 mL) was added TFA (3 mL). The reaction mixture was stirred for 1 h. The solvent was removed, azetroping excess TFA with 1:1 DCM/heptane, to give the title compound (containing 50% TFA) (474 mg, 149%) as a pale yellow oil. LCMS (Method 5): did not ionise, RT 0.12 minutes.

### Intermediate 16

### Ethyl 2-[6-(tert-butoxycarbonylamino)-3-chloropyridazin-4-yl]acetate

HCl in 1,4-dioxane (4M, 4.6 mL, 18.6 mmol) was added to a solution of Intermediate 2 (3.56 g, 12.4 mmol) in ethanol (44.5 mL) at r.t. The reaction mixture was stirred at r.t. for 22 h, then diluted with EtOAc (100 mL) and washed with saturated aqueous NaHCO₃ solution (30 mL). The aqueous phase was extracted with EtOAc (3×50 mL). The combined organic phases were dried over sodium sulfate, then filtered and concentrated to dryness. The residue was purified by silica column chromatography, eluting with 10-30% EtOAc in heptane, to afford the title compound (3.17 g, 81%) as an off-white solid. LCMS (Method 5): [M-tBu+H]⁺ m/z 260, RT 1.84 minutes.

### Intermediate 17

### Ethyl 2-[6-(tert-butoxycarbonylamino)-3-chloropyridazin-4-yl]-4,4-difluorobut-2-enoate

1-Ethoxy-2,2-difluoroethanol (90%, 3.0 mL, 24.5 mmol) was added to a solution of Intermediate 16 (1.55 g, 4.91 mmol) and triethylamine (2.7 mL, 19.6 mmol) in 1,4-dioxane (10 mL) at r.t. The reaction mixture was stirred at 100° C. for 16 h in a pressure round-bottomed flask, then concentrated to dryness. The residue was purified by silica column chromatography, eluting with 10-20% EtOAc in heptane, to afford the title compound (1.39 g, 62%) as a yellow gum. LCMS (Method 6): [M-tBu+H]⁺ m/z 322, RT 3.07 minutes.

### Intermediate 18

### Ethyl 2-[6-(tert-butoxycarbonylamino)-3-chloropyridazin-4-yl]-4,4-difluorobutanoate

Sodium borohydride (27 mg, 0.719 mmol) was added to a solution of Intermediate 17 (255 mg, 0.675 mmol) in DCM (3.5 mL) at −4° C., then ethanol (3.5 mL) was added. The reaction mixture was maintained at −4° C. with stirring for 15 minutes. Saturated aqueous NH₄Cl solution (30 mL) was added and the reaction mixture was stirred at −4° C. for 15 minutes, then diluted with water (30 mL) and DCM (60 mL). The layers were separated and the aqueous layer was extracted with DCM (2×30 mL). The combined organic phases were filtered through a phase separator filter, then concentrated to dryness under vacuum, to afford the title compound (261 mg, 83%) as a pale yellow gum. LCMS (Method 5): [M-tBu+H]⁺ m/z 324, RT 1.95 minutes.

### Intermediate 19

### Ethyl 2-(6-amino-3-chloropyridazin-4-yl)-4,4-difluorobutanoate

TFA (1.1 mL, 14.4 mmol) was added to a solution of Intermediate 18 (281 mg, 0.741 mmol) in DCM (1.1 mL) at r.t. The reaction mixture was stirred at r.t. for 3 h, then diluted with DCM (10 mL), and saturated aqueous NaHCO₃ solution (10 mL) was added. The layers were separated, and the aqueous phase was re-extracted with DCM (2×10 mL). The organic phase was dried over sodium sulfate, then concentrated to dryness under vacuum, to give the title compound (207 mg, 86%) as a tan gum. LCMS (Method 5): [M+H]⁺ m/z 280, RT 1.54 minutes.

### Intermediate 20

### Ethyl 2-{2-[(S)-benzyloxycarbonylamino(4,4-difluorocyclohexyl)methyl]-6-chloroimidazo[1,2-b]pyridazin-7-yl}-4,4-difluorobutanoate

2,6-Dimethylpyridine (220 μL, 1.89 mmol) was added to a stirred mixture of Intermediate 19 (203 mg, 0.726 mmol) and benzyl N-[(1S)-3-bromo-1-(4,4-difluoro-cyclohexyl)-2-oxopropyl]carbamate (381 mg, 0.944 mmol) in 1,4-dioxane (2.9 mL). The reaction mixture was stirred at 80° C. for 4 h in a pressure vial. The residue was diluted with EtOAc (50 mL), washed with water (20 mL) and brine (10 mL), then dried over sodium sulfate, and concentrated to dryness under vacuum. The crude residue was purified by silica column chromatography, eluting with 10-100% EtOAc in heptane, to afford the title compound (355 mg, 84%) as a tan gum. LCMS (Method 5): [M+H]⁺ m/z 585, RT 2.11 minutes.

### Intermediate 21

### Ethyl 2-{2-[(S)-amino(4,4-difluorocyclohexyl)methyl]imidazo[1,2-b]pyridazin-7-yl}-4,4-difluorobutanoate hydrochloride

Intermediate 20 (165 mg, 0.282 mmol) was dissolved in ethanol (7 mL) and 1M aqueous HCl (1.4 mL, 1.41 mmol). The reaction mixture was cycled between vacuum and nitrogen three times, then 10% Pd/C (50% wet, 120 mg, 0.0564 mmol) was added. The reaction mixture was stirred under a hydrogen atmosphere for 3 h at r.t., then filtered through a pad of Celite®. The solids were washed with EtOH (50 mL) and EtOAc (50 mL). The combined filtrates were concentrated to dryness, and azeotroped with toluene (3×3 mL) under vacuum, to afford the title compound (16 mg, 100%) as a tan solid. LCMS (Method 5): [M+H]⁺ m/z 417, RT 1.60 minutes.

### Intermediate 22

### Ethyl 1-(3,6-dichloropyridazin-4-yl)-4,4-difluorocyclohexanecarboxylate

n-Butyllithium in hexane (2.5M, 7.2 mL, 18.0 mmol) was added dropwise to a stirred solution of dicyclohexylamine (3.6 mL, 18.1 mmol) in anhydrous toluene (24 mL) at 0° C. under nitrogen. The resultant white suspension was stirred at 0° C. for 20 minutes, then at 20° C. for 30 minutes, then cooled to 0° C. Ethyl 4,4-difluorocyclohexanecarboxylate (2.19 mL, 12.53 mmol) was added dropwise. The resultant yellow-orange hazy suspension was stirred at 0° C. for 15 minutes, then at 20° C. for 15 minutes, then added to a dried flask charged with 4-bromo-3,6-dichloropyridazine (95%, 2.50 g, 10.4 mmol) and methanesulfonato(tri-t-butylphosphino)(2′-methylamino-1,1′-biphenyl-2-yl)-palladium(II) [P(t-Bu)3 Palladacycle Gen. 4] (CAS no. 1621274-11-0) (620 mg, 1.06 mmol) under nitrogen. The mixture was stirred at 20° C. under nitrogen for 18 h, then diluted with DCM (50 mL) and quenched with 0.5M aqueous hydrochloric acid (50 mL). The suspension was filtered through a Celite® pad, and washed with DCM (50 mL) and water (30 mL). The organic phase was separated, and the aqueous layer was extracted with DCM (2×100 mL). The combined organic extracts were washed with 0.5M aqueous hydrochloric acid (50 mL) and brine (50 mL), then dried over magnesium sulfate and concentrated in vacuo. The residue was purified by silica column chromatography, eluting with a gradient of 0-30% EtOAc in heptane, to afford the title compound (76% purity) (859 mg, 19%) as an orange waxy solid. LCMS (Method 1): [M+H]⁺ m/z 339/341/343, RT 2.93 minutes.

### Intermediate 23

### Ethyl 1-[6-(tert-butoxycarbonylamino)-3-chloropyridazin-4-yl]-4,4-difluorocyclohexanecarboxylate

Intermediate 22 (79%, 900 mg, 2.10 mmol), tert-butyl carbamate (296.5 mg, 2.53 mmol) and cesium carbonate (972 mg, 1.59 mmol) were suspended in anhydrous 1,4-dioxane (10.6 mL) and purged with nitrogen whilst sonicating for 5 minutes. Tris-(dibenzylideneacetone)dipalladium(0) (77.4 mg, 84.5 μmol) and Xantphos (97.2 mg, 0.17 mmol) were added. The mixture was purged with nitrogen whilst sonicating for 5 minutes, then sealed under nitrogen, heated at 90° C. for 18 h and diluted with EtOAc (20 mL). The solids were removed by filtration through Celite®, washing with EtOAc (3×30 mL). The filtrate was concentrated in vacuo. The residue was purified by silica column chromatography, eluting with a gradient of 0-40% EtOAc in heptane, to afford the title compound (91% purity) (597 mg, 62%) as a yellow-brown powder. LCMS (Method 4): [M+H]⁺ m/z 420/422, RT 2.06 minutes.

### Intermediate 24

### Ethyl 1-(6-amino-3-chloropyridazin-4-yl)-4,4-difluorocyclohexanecarboxylate

TFA (2 mL, 26.9 mmol) was added to a stirred solution of Intermediate 23 (91%, 615 mg, 1.33 mmol) in DCM (14 mL). The reaction mixture was stirred at 20° C. for 4 h, then quenched with saturated aqueous sodium hydrogen carbonate solution (30 mL) and diluted with DCM (30 mL). The biphasic mixture was stirred at 20° C. for 5 minutes, then the organic phase was separated and washed with saturated aqueous sodium hydrogen carbonate solution (20 mL). The combined aqueous washings were extracted with DCM (2×30 mL). The combined organic extracts were washed with brine (30 mL) and dried over magnesium sulfate, then concentrated in vacuo, to afford the title compound (90% purity) (474 mg, 100%) as an orange-yellow powder. LCMS (Method 4): [M+H]⁺ m/z 320/322, RT 1.73 minutes.

### Intermediate 25

### Ethyl 1-{2-[(S)-benzyloxycarbonylamino(4,4-difluorocyclohexyl)methyl]-6-chloroimidazo[1,2-b]pyridazin-7-yl}-4,4-difluorocyclohexanecarboxylate

A suspension of Intermediate 24 (90%, 474 mg, 1.33 mmol), benzyl N-[(1S)-3-bromo-1-(4,4-difluorocyclohexyl)-2-oxopropyl]carbamate (639 mg, 1.58 mmol) and sodium hydrogen carbonate (247 mg, 2.94 mmol) in IPA (7.3 mL) was sealed under nitrogen and heated at 65° C. for 18 h. After cooling, the mixture was diluted with water (30 mL) and saturated aqueous sodium hydrogen carbonate solution (10 mL), then extracted with EtOAc (3×30 mL). The combined organic extracts were washed with brine (30 mL), dried over magnesium sulfate and concentrated in vacuo. The residue was purified by silica column chromatography, eluting with a gradient of 5-60% EtOAc in heptane, to afford the title compound (90% purity) (788 mg, 85%) as an orange viscous oil. LCMS (Method 4): [M+H]⁺ m/z 625/627, RT 2.17 minutes.

### Intermediate 26

### Ethyl 1-{2-[(S)-amino(4,4-difluorocyclohexyl)methyl]imidazo[1,2-b]pyridazin-7-yl}-4,4-difluorocyclohexanecarboxylate hydrochloride

A suspension of Intermediate 25 (90%, 788 mg, 1.13 mmol) and 10% palladium on carbon (50% water wet, 188 mg, 0.09 mmol) in 4:1 EtOH/water (17.5 mL) was placed under a hydrogen atmosphere and stirred at 20° C. for 18 h. The reaction mixture was filtered through Celite® and washed with EtOH (5×50 mL), then concentrated in vacuo, to afford the title compound (95% purity) (536 mg, 91%) as a tan powder. LCMS (Method 1): [M+H]⁺ m/z 457, RT 2.92 minutes.

### Intermediate 27

### Ethyl 1-{2-(S)-(4,4-difluorocyclohexyl)[(4-methyl-1,2,5-oxadiazole-3-carbonyl)amino]-methyl}imidazo[1,2-b]pyridazin-7-yl)-4,4-difluorocyclohexanecarboxylate

HATU (400 mg, 1.05 mmol) was added to a stirred solution of Intermediate 26 (95%, 400 mg, 0.77 mmol), 4-methyl-1,2,5-oxadiazole-3-carboxylic acid (124 mg, 0.97 mmol) and DIPEA (0.35 mL, 2.00 mmol) in anhydrous DMF (4 mL). The reaction mixture was stirred at 20° C. under nitrogen for 18 h, then quenched with saturated aqueous sodium hydrogen carbonate solution (10 mL) and diluted with water (10 mL). The material was extracted with EtOAc (3×30 mL). The combined organic extracts were washed successively with 0.5M aqueous hydrochloric acid/brine (1:1, 20 mL) and water/brine (1:1, 3×20 mL). The organic phase was dried over magnesium sulfate, then filtered and concentrated in vacuo. The residue was purified by silica column chromatography, eluting with a gradient of 0-60% EtOAc in heptane, to afford the title compound (93% purity) (321 mg, 68%) as an off-white powder. LCMS (Method 1): [M+H]⁺ m/z 567, RT 3.41 minutes.

### Intermediate 28

### 1-(2-{(S)-(4,4-Difluorocyclohexyl)[(4-methyl-1,2,5-oxadiazole-3-carbonyl)amino]-methyl}imidazo[1,2-b]pyridazin-7-yl)-4,4-difluorocyclohexanecarboxylic acid

Aqueous lithium hydroxide (1M, 2.8 mL, 2.8 mmol) was added to a stirred solution of Intermediate 27 (93%, 320 mg, 0.53 mmol) in MeOH (11 mL). The mixture was heated at 50° C. for 24 h. After cooling, the volatiles were removed in vacuo, and the aqueous residue was diluted with water (30 mL). The pH was adjusted to pH 1 with 1M aqueous hydrochloric acid, and the material was extracted with EtOAc (3×30 mL). The combined organic extracts were washed with brine (30 mL) and dried over magnesium sulfate, then filtered and concentrated in vacuo, to afford the title compound (92% purity) (330 mg, quantitative) as an off-white powder. LCMS (Method 5): [M+H]⁺ m/z 539, RT 1.91 minutes.

### Intermediate 29

### Methyl 2-[6-(tert-butoxvcarbonylamino)pyridazin-4-yl]-4,4-difluorobutanoate

To a solution of Intermediate 4 (2.20 g, 6.05 mmol) in ethanol (60 mL) under N₂ was added 10% palladium on carbon (220 mg, 0.207 mmol). The reaction flask was placed under an atmosphere of H₂ and the reaction mixture was stirred for 20 h at r.t., then filtered through a pad of Celite® and concentrated in vacuo. The crude material was purified by flash column chromatography, eluting with a gradient of 0-50% EtOAc in isohexane, to afford the title compound (278 mg, 14%) as a colourless amorphous solid. LCMS (Method 7): [M+H]⁺ m/z 332.0, RT 2.03 minutes.

### Intermediate 30 Methyl 2-(6-aminopyridazin-4-yl)-4,4-difluorobutanoate

To a solution of Intermediate 29 (345 mg, 1.04 mmol) in DCM (4.2 mL) was added TFA (4.2 mL). The reaction mixture was heated at 40° C. for 30 minutes, then concentrated in vacuo. The residue was dissolved in DCM (20 mL) and saturated aqueous sodium bicarbonate solution (20 mL). The aqueous layer was extracted with DCM (2×20 mL). The combined organic extracts were passed through a phase separator, then concentrated in vacuo, to give the title compound (157 mg, 65%) as an off-white amorphous solid. δ_{H} (300 MHz, DMSO-d₆) 8.41 (1H, d, J 2.0 Hz), 6.68 (1H, d, J 2.0 Hz), 6.42 (2H, br s), 6.10 (1H, tt, J 56.0, 4.5 Hz), 3.86 (1H, t, J 7.5 Hz), 3.65 (3H, s), 2.72-2.54 (1H, m), 2.41-2.21 (1H, m). LCMS (Method 7): [M+H]⁺ m/z 232.0, RT 0.73 minutes.

### Intermediate 31

### Methyl 2-{2-[(S)-benzyloxycarbonylamino(4,4-difluorocyclohexyl)methyl]imidazo[1,2-b]pyridazin-7-yl}-4,4-difluorobutanoate

To a solution of Intermediate 30 (150 mg, 0.649 mmol) and benzyl N-[(1S)-3-bromo-1-(4,4-difluorocyclohexyl)-2-oxopropyl]carbamate (315 mg, 0.779 mmol) in IPA (3.2 mL) was added sodium bicarbonate (82 mg, 0.98 mmol). The reaction mixture was stirred at 80° C. overnight, then concentrated in vacuo. The residue was dissolved in EtOAc (20 mL). The organic layer was washed with water (20 mL), and the aqueous layer was extracted with EtOAc (2×20 mL). The combined organic extracts were passed through a phase separator and concentrated in vacuo. The crude material was purified by silica column chromatography, eluting with a gradient of 0-80% EtOAc in isohexane, to give the title compound (151 mg, 43%) as an off-white amorphous solid. LCMS (Method 7): [M+H]⁺ m/z 537.0, RT 2.70 minutes.

### Intermediate 32

### 2-{2-[(S-Benzyloxycarbonylamino(4,4-difluorocyclohexyl)methyl]imidazo[1,2-b]-pyridazin-7-yl}-4,4-difluorobutanoic acid

To a solution of Intermediate 31 (151 mg, 0.281 mmol) in THE (1.2 mL) and water (0.3 mL) was added lithium hydroxide monohydrate (24 mg, 0.56 mmol). The reaction mixture was stirred at 40° C. for 1 h, then neutralised with 2M aqueous hydrochloric acid (0.28 mL) and concentrated in vacuo, to give the title compound (146 mg, 99%) as a yellow amorphous solid. LCMS (Method 7): [M+H]⁺ m/z 523.0, RT 1.76 minutes.

### Intermediate 33

### Benzyl N-{(S)-(4,4-difluorocyclohexyl)[7-(3,3-difluoro-1-{[(1S)-2,2,2-trifluoro-1-methylethyl]carbamoyl}propyl)imidazo[1,2-b]pyridazin-2-yl]methyl}carbamate

To a solution of Intermediate 32 (146 mg, 0.279 mmol), (S)-2-amino-1,1,1-trifluoropropane hydrochloride (53 mg, 0.34 mmol) and DIPEA (0.15 mL, 0.84 mmol) in DMF (2.8 mL) was added HATU (131 mg, 0.335 mmol). The reaction mixture was stirred at r.t. for 10 minutes, then concentrated in vacuo. The residue was dissolved in DCM (20 mL) and washed with water (20 mL). The aqueous layer was extracted with DCM (2×20 mL), and the combined organic extracts were passed through a phase separator and concentrated in vacuo. The crude material was purified by silica column chromatography, eluting with a gradient of 0-60% EtOAc in isohexane, to give the title compound (176 mg, 98%) as an off-white amorphous solid. LCMS (Method 7): [M+H]⁺ m/z 618.0, RT 2.76 minutes.

### Intermediate 34

### 2-{2-[(S)-amino(4,4-difluorocyclohexyl)methyl]imidazo[1,2-b]pyridazin-7-yl}-4,4-difluoro-N-[(1S)-2,2,2-trifluoro-1-methylethyl]butanamide

To a solution of Intermediate 33 (176 mg, 0.274 mmol) in ethanol (2.7 mL) under N₂ was added 10% palladium on carbon (88 mg, 0.08 mmol). The reaction flask was placed under an atmosphere of H₂ and the reaction mixture was stirred at r.t. for 3 h, then filtered through a pad of Celite® and concentrated in vacuo, to give the title compound (109 mg, 82%) as a green oil. LCMS (Method 7): [M+H]⁺ m/z 484.0, RT 2.12 minutes.

### Intermediate 35

### 5-(Dicyclopropylmethyl)imidazolidine-2,4-dione

A stirred mixture of diammonium carbonate (47.10 g, 0.501 mol), 2,2-dicyclopropylacetaldehyde (95%, 26.10 g, 0.200 mol) and potassium cyanide (13.09 g, 0.201 mol) in a mixture of ethanol (140 mL) and water (140 mL) was heated at 60° C. for 18 h. To the cooled reaction mixture were added 2N HCl (200 mL), then 6N HCl (100 mL) partwise. Additional 2N HCl (60 mL) was added, and the mixture was stirred at r.t. for 1 h. Additional 2N HCl (50 mL) was added to the mixture and the solid was filtered off, then washed with water (2×200 mL) and dried, to afford the title compound (95% purity) (32.81 g, 80%) as a white solid. δ_{H} (500 MHz, DMSO-d₆) 10.58 (s, 1H), 8.04 (s, 1H), 4.05 (d, J 1.5 Hz, 1H), 0.91-0.81 (m, 1H), 0.82-0.73 (m, 1H), 0.52-0.36 (m, 3H), 0.36-0.23 (m, 3H), 0.23-0.17 (m, 1H), 0.13-0.07 (m, 1H), 0.06-0.00 (m, 1H). LCMS (Method 3): [M+H]⁺ m/z 195, RT 1.72 minutes.

### Intermediate 36

### 2-(Benzyloxycarbonylamino)-3,3-dicyclopropylpropanoic acid

To a stirred solution of Intermediate 35 (95%, 1.00 g, 4.89 mmol) in 1,4-dioxane (6 mL) was added 5M aqueous sodium hydroxide solution (6.0 mL, 30.0 mmol). The mixture was heated at 100° C. for 18 h, then 1,4-dioxane (6 mL) and water (6 mL) were added, and the mixture was heated at 120° C. for two days. To the cooled reaction mixture were added TBME (10 mL) and water (10 mL). The two-phase mixture was filtered. To the filtrate were added 6N HCl (6 mL) and TBME (10 mL). The undissolved solid was filtered off. To the filtrate was added TBME (10 mL). The layers were separated, and the aqueous layer was washed with TBME (3×10 mL). To the aqueous layer was added 5N aqueous NaOH solution (0.5 mL), and the pH of the solution was adjusted to pH 7 using 6N HCl/5N aqueous NaOH solution. To the aqueous solution (˜40 mL) were added THE (20 mL), then NaHCO₃ (1.01 g), then disodium carbonate (1.01 g, 9.53 mmol), then 1-(benzyloxycarbonyloxy)pyrrolidine-2,5-dione (0.90 g, 3.61 mmol) at r.t. The mixture was stirred at r.t. for 2.5 days, then TBME (20 mL) was added, followed by water (30 mL). The organic layer was separated off. To the aqueous layer was added water (10 mL). The aqueous layer was washed with TBME (10 mL), then filtered and washed with TBME (10 mL). The pH of the aqueous layer was adjusted to pH 3 using 6N HCl (˜4 mL). Crystals from a previous batch were seeded, then the flask was externally cooled and left to stand for 2 h. The contents were filtered off, then washed with water (2×10 mL) and dried, to afford the title compound (719 mg, 49%) as a white solid. δ_{H} (500 MHz, DMSO-d₆) 12.53 (s, 1H), 7.47 (d, J 8.9 Hz, 1H), 7.41-7.28 (m, 5H), 5.11-5.01 (m, 2H), 4.19 (dd, J 8.9, 4.4 Hz, 1H), 1.03-0.92 (m, 1H), 0.85-0.74 (m, 1H), 0.57-0.49 (m, 1H), 0.49-0.43 (m, 1H), 0.41-0.20 (m, 4H), 0.19-0.01 (m, 3H). LCMS (Method 3): [M+H]⁺ m/z 304, RT 3.13 minutes.

### Intermediate 37

tert-Butyl 4-(benzyloxycarbonylamino)-5,5-dicyclopropyl-3-oxopentanoate Intermediate 36 (1.00 g, 3.30 mmol) was dissolved in anhydrous THE (12 mL) under nitrogen and DIPEA (650 μL, 3.72 mmol) was added, followed by HATU (1.40 g, 3.68 mmol). The mixture was stirred under nitrogen for 1.5 h. In a separate flask, a solution of LDA in THF/heptanes/ethylbenzene (2M, 7.0 mL, 14.0 mmol) and anhydrous THE (10 mL) were placed under nitrogen and cooled to −78° C. tert-Butyl acetate (1.9 mL, 14.2 mmol) was added dropwise over approximately 5 minutes. The resulting solution was stirred at −78° C. under nitrogen for 1 h, then the separate solution of activated acid was added dropwise, maintaining the internal temperature below −60° C. The resulting mixture was stirred at −78° C. under nitrogen for 1 h, then quenched at −78° C. by the addition of saturated aqueous NH₄Cl solution (50 mL). The mixture was allowed to warm to r.t., then extracted with EtOAc (100 mL). The organic layer was washed with brine (50 mL), then dried (Na₂SO₄) and concentrated to dryness under vacuum. The residue was purified by flash column chromatography, eluting with 0-20% EtOAc in heptane, to afford the title compound (85% purity) (1.15 g, 74%) as a pale yellow oil. δ_{H} (400 MHz, CDCl₃) 7.40-7.29 (m, 5H), 5.64 (d, J 8.8 Hz, 1H), 5.11 (s, 2H), 4.68 (dd, J 8.9, 2.7 Hz, 1H), 3.50 (s, 2H), 1.70-1.59 (m, 1H), 1.46 (s, 9H), 0.79-0.71 (m, 2H), 0.67 (dt, J 13.0, 6.9 Hz, 1H), 0.58-0.44 (m, 2H), 0.39 (tt, J 9.4, 4.8 Hz, 1H), 0.26 (dd, J 9.2, 4.3 Hz, 2H), 0.17 (ddt, J 13.4, 9.2, 4.9 Hz, 2H). LCMS (Method 5): [M-tBu+H]⁺ m/z 346, RT 2.12 minutes.

### Intermediate 38

### Benzyl N-[3-bromo-1-(dicyclopropylmethyl)-2-oxopropyl]carbamate

Intermediate 37 (85%, 1.15 g, 2.43 mmol) was dissolved in methanol (5 mL) and 2,6-dimethylpyridine (30 μL, 0.258 mmol) was added, followed by NBS (440 mg, 2.47 mmol). The reaction mixture was stirred under nitrogen for 18 h, diluted with EtOAc (50 mL), washed with a 50% mixture of water and brine (2×40 mL), and washed with brine (20 mL), then dried (Na₂SO₄) and concentrated to dryness under vacuum. The residue was dissolved in toluene (11 mL) and TFA (1.4 mL, 18.8 mmol) was added. The reaction mixture was stirred at 80° C. under nitrogen for 2.5 h, then allowed to cool to r.t. and concentrated to dryness under vacuum. The residue was purified by flash column chromatography, eluting with 30-100% DCM in heptane, followed by 0-100% EtOAc in DCM, to afford the title compound (87% purity) (780 mg, 73%) as a colourless solid. δ_{H} (500 MHz, CDCl₃) 7.45-7.28 (m, 5H), 5.59 (d, J 8.1 Hz, 1H), 5.12 (s, 2H), 4.81 (dd, J 8.4, 3.2 Hz, 1H), 4.26-4.08 (m, 2H), 0.78-0.61 (m, 3H), 0.61-0.48 (m, 3H), 0.43 (ddd, J 13.6, 9.6, 5.2 Hz, 1H), 0.27 (dq, J 10.9, 6.0, 5.4 Hz, 2H), 0.17 (dt, J 8.8, 4.7 Hz, 2H).

LCMS (Method 6): [M+H]⁺ m/z 380/382, RT 3.21 minutes.

### Intermediate 39

### Ethyl 2-{2-[1-(benzyloxycarbonylamino)-2,2-dicyclopropylethyl]-6-chloroimidazo[1,2-b]pyridazin-7-yl}-4,4-difluorobutanoate

Intermediate 19 (365 mg, 1.305 mmol) and Intermediate 38 (90%, 606.6 mg, 1.435 mmol) were dissolved in dry THE (10 mL), and sodium hydrogen carbonate (328.9 mg, 3.915 mmol) was added. The reaction mixture was heated with stirring at 70° C. for 18 h, then cooled to r.t. The resulting solids were filtered off and washed with DCM. The filtrate was concentrated under vacuum. The material was purified by silica column chromatography, eluting with a gradient of 0-40% ethyl acetate in heptane, to afford the title compound (566 mg, 77%) as a yellow/orange foam. LCMS (Method 5): [M+H]⁺ m/z 561.2, RT 2.16 minutes.

### Intermediate 40

### Ethyl 2-{2-[(1S)-1-amino-2,2-dicyclopropylethyl]imidazo[1,2-b]pyridazin-7-yl}-4,4-difluorobutanoate hydrochloride

Intermediate 39 (275 mg, 0.490 mmol) was dissolved in EtOH (15 mL) and 1M aqueous HCl (2.45 mL, 2.450 mmol) was added, followed by 10% Pd/C (50% wet) (5.0%, 52.16 mg, 0.0245 mmol). The reaction mixture was stirred under 1 atmosphere of H₂ for 3 h, then filtered through Celite® and concentrated under vacuum to remove the solvent. The residue was azeotroped with toluene (3×3 mL) under vacuum to afford the title compound (190 mg, 90%) as a yellow solid. LCMS (Method 5): [M+H]⁺ m/z 393.2, RT 1.63 minutes.

### Intermediate 41

### Ethyl 2-(2-{(1S)-2,2-dicyclopropyl-1-[(4-methyl-1,2,5-oxadiazole-3-carbonyl)amino]-ethyl}imidazo[1,2-b]pyridazin-7-yl)-4,4-difluorobutanoate

Intermediate 40 (190 mg, 0.443 mmol) and 4-methyl-1,2,5-oxadiazole-3-carboxylic acid (68 mg, 0.531 mmol) were stirred in anhydrous DMF (3 mL). DIPEA (232 μL, 1.329 mmol) was added, followed by HATU (202 mg, 0.531 mmol). The mixture was stirred for 3 h at r.t., then diluted with EtOAc (30 mL) and washed with water (3×20 mL). The organic phase was dried over sodium sulfate, then filtered and concentrated to dryness under vacuum. The crude residue was purified by flash column chromatography, eluting with a gradient of 0-40% ethyl acetate in heptane, to afford the title compound (156 mg, 71%) as a yellow solid. LCMS (Method 5): [M+H]*m/z 503.2, RT 2.06 minutes.

### Intermediate 42

### 2-(2-{(1S)-2,2-Dicyclopropyl-1-[(4-methyl-1,2,5-oxadiazole-3-carbonyl)amino]ethyl}-imidazo[1,2-b]pyridazin-7-yl)-4,4-difluorobutanoic acid

A mixture of lithium hydroxide monohydrate (29 mg, 0.679 mmol) and Intermediate 41 (155 mg, 0.308 mmol) in THE (4 mL), water (0.6 mL) and methanol (1.5 mL) was stirred at r.t. for 16 h. The reaction mixture was concentrated to low volume under vacuum, then diluted with ethyl acetate (50 mL). Aqueous HCl (0.5M) was added until pH 3 was achieved. The layers were separated, and the aqueous layer was extracted with EtOAc (2×30 mL). The organic phase was washed with brine and dried over sodium sulfate, then filtered and concentrated to dryness under vacuum, to afford the title compound (142 mg, 97%) as a yellow solid. LCMS (Method 5): [M+H]⁺ m/z 475.2, RT 1.88 minutes.

### Intermediate 43

### Lithium 2-{2-[(S)-benzyloxycarbonylamino(4,4-difluorocyclohexyl)methyl]imidazo[1,2-b]pyridazin-7-yl}-4,4-difluorobutanoate

A mixture of lithium hydroxide monohydrate (56 mg, 1.29 mmol) and Intermediate 58 (324 mg, 0.589 mmol) in THE (7 mL), water (1.5 mL) and methanol (3 mL) was stirred at r.t. for 2 h. The reaction mixture was concentrated to dryness in vacuum, re-dissolved in toluene (5 mL) and re-concentrated in vacuum. The residue was re-dissolved in toluene (5 mL) and re-concentrated to dryness in vacuum twice more, to give the title compound (311 mg, 95%) as a yellow solid. LCMS (Method 5): [M+H]⁺ m/z 523, RT 1.88 minutes.

### Intermediate 44

### Benzyl N-[(S)-(4,4-difluorocyclohexyl){7-[3,3-difluoro-1-(2,2,2-trifluoroethyl-carbamoyl)propyl]imidazo[1,2-b]pyridazin-2-yl}methyl]carbamate

HATU (671 mg, 1.77 mmol) was added to a mixture of Intermediate 43 (311 mg, 0.589 mmol) and DIPEA (308 μL, 1.77 mmol) in anhydrous DMF (8 mL) at r.t. The reaction mixture was stirred at r.t. for 5 minutes, then 2,2,2-trifluoroethanamine (139 μL, 1.77 mmol) was added. The reaction mixture was stirred at r.t. for 16 h, then diluted with EtOAc (50 mL) and washed with water (3×60 mL). The organic phase was dried over sodium sulfate, then filtered and concentrated to dryness. The resulting crude material was purified by FCC (Biotage Isolera, SiO₂, gradient elution 10-60% EtOAc:heptanes) to give the title compound (246 mg, 68%) as a yellow gum. LCMS (Method 5): [M+H]⁺ m/z 604, RT 1.99 minutes.

### Intermediate 45

### Benzyl N-[(S)-(4,4-difluorocyclohexyl){7-[3,3-difluoro-1-(2,2,2-trifluoroethyl-carbamothioyl)propyl]imidazo[1,2-b]pyridazin-2-yl}methyl]carbamate

Lawesson's Reagent (85 mg, 0.210 mmol) was added to a solution of Intermediate 44 (211 mg, 0.350 mmol) in anhydrous 1,4-dioxane (2 mL) under nitrogen. The reaction mixture was stirred at 90° C. for 22 h, then diluted with EtOAc (60 mL) and washed with saturated aqueous NaHCO₃ solution (20 mL). The aqueous layer was extracted with EtOAc (2×30 mL). The combined organic phases were washed with brine and dried over sodium sulfate, then filtered and concentrated to dryness in vacuum. The resulting crude material was purified by FCC (Biotage Isolera, gradient elution 10-60% EtOAc:heptanes) to give the title compound (154 mg, 71%) as a tan gum. LCMS (Method 5): [M+H]⁺ m/z 620, RT 2.07 minutes.

### Intermediate 46

### Benzyl N-[(S)-(4,4-difluorocyclohexyl)(7-{3,3-difluoro-1-[1-(2,2,2-trifluoroethyl)-tetrazol-5-yl]propyl}imidazo[1,2-b]pyridazin-2-yl)methyl]carbamate

Triphenylphosphine (98 mg, 0.373 mmol) was added to a solution of Intermediate 45 (154 mg, 0.249 mmol), DIAD (73 μL, 0.373 mmol) and azido(trimethyl)silane (98 μL, 0.746 mmol) in anhydrous THE (0.8 mL) at r.t. The reaction mixture was stirred at r.t. for 3 days, then diluted with EtOAc (40 mL) and washed with saturated aqueous NaHCO₃ solution. The aqueous phase was re-extracted with EtOAc (3×30 mL). The organic phase was dried over sodium sulfate, then filtered and concentrated to dryness in vacuum. The resulting crude material was purified by FCC (Biotage Isolera, SiO₂, gradient elution 10-50% EtOAc:heptanes) to give the title compound (122 mg, 62%) as a tan gum. LCMS (Method 6): [M+H]⁺ m/z 629, RT 3.26 minutes.

### Intermediate 47

### (S)-(4,4-Difluorocyclohexyl)(7-{3,3-difluoro-1-[1-(2,2,2-trifluoroethyl)tetrazol-5-yl]-propyl}imidazo[1,2-b]pyridazin-2-yl)methanamine

Intermediate 46 (122 mg, 0.194 mmol) was dissolved in EtOH (14 mL) and water (1.4 mL). The reaction mixture was cycled three times between vacuum and nitrogen, then palladium on carbon (10% loading, 50% water) (5.0%, 83 mg, 0.0388 mmol) was added. The reaction mixture was cycled three times between vacuum and hydrogen, then stirred under a hydrogen atmosphere for 22 h at r.t. The reaction vessel was purged of hydrogen by cycling three times between vacuum and nitrogen. The reaction mixture was filtered through a pad of Celite®, and the solids were washed with EtOH (3×30 mL). The combined filtrates were concentrated to dryness, then azeotroped with toluene (3×3 mL) under vacuum, to give the title compound (96 mg, 100%) as a pale tan gum. LCMS (Method 5): [M+H]⁺ m/z 495, RT 2.00 minutes.

### Intermediate 48

### Benzyl N-[(S)-(7-chloroimidazo[1,2-b]pyridazin-2-yl)(4,4-difluorocyclohexyl)methyl]-carbamate

A solution of benzyl N-[(1S)-3-bromo-1-(4,4-difluorocyclohexyl)-2-oxopropyl]-carbamate (1.65 g, 4.08 mmol), 5-chloropyridazin-3-amine trifluoroacetic acid salt (900 mg, 3.670 mmol) and sodium bicarbonate (650 mg, 7.74 mmol) in 2-propanol (40 mL) was stirred at 80° C. for 18 h. The reaction mixture was concentrated in vacuo. The residue was purified by flash column chromatography, eluting with a gradient of 0-35% EtOAc in isohexane, then freeze-dried from water/acetonitrile, to give the title compound (758 mg, 47%) as a light-coloured powder. δ_{H} (400 MHz, DMSO-d₆) 8.62 (d, J 2.3 Hz, 1H), 8.37 (d, J 2.4 Hz, 1H), 8.18 (s, 1H), 7.84 (d, J 9.4 Hz, 1H), 7.39-7.27 (m, 5H), 5.09-4.98 (m, 2H), 4.74 (dd, J 9.4, 7.0 Hz, 1H), 2.00 (s, 3H), 1.85-1.53 (m, 4H), 1.42-1.15 (m, 2H).

### Intermediate 49

### O-tert-Butyl O³-ethyl 4-{2-[(S)-benzyloxycarbonylamino(4,4-difluorocyclohexyl)-methyl]imidazo[1,2-b]pyridazin-7-yl}-2,5-dihydropyrrole-1,3-dicarboxylate

Bis(pinacolato)diboron (370 mg, 1.46 mmol), potassium acetate (325 mg, 3.28 mmol), tris(dibenzylideneacteone)dipalladium(0) (120 mg, 0.13 mmol), XPhos (130 mg, 0.26 mmol) and O-tert-butyl O³-ethyl 4-(trifluoromethylsulfonyloxy)-2,5-dihydro-pyrrole-1,3-dicarboxylate (WO 2020/261141, Intermediate 4) (513 mg, 1.3 mmol) were placed in a vial, followed by anhydrous 1,4-dioxane (2.6 mL). The reaction mixture was thoroughly degassed, then heated at 100° C. for 2 h. Intermediate 48 (460 mg, 1.06 mmol) was added, followed by a solution of potassium phosphate tribasic (425 mg, 2.00 mmol) in water (1.1 mL). The mixture was degassed again and heated at 90° C. for 1 h, then diluted with EtOAc (50 mL) and washed with water (2×50 mL). The aqueous layer was re-extracted with EtOAc (50 mL). The combined organic extracts were passed down a hydrophobic frit and concentrated in vacuo. The residue was purified by flash column chromatography, eluting with a gradient of 0-100% EtOAc in isohexane, then freeze-dried from water/acetonitrile, to give the title compound (486 mg, 72%) as a pale yellow solid. LCMS (Method 7): [M+H]⁺ m/z 640, RT 2.77 minutes.

### Intermediate 50

### Lithium 4-{2-[(S)-benzyloxycarbonylamino(4,4-difluorocyclohexyl)methyl]imidazo[1,2-b]pyridazin-7-yl}-1-(tert-butoxvcarbonyl)-2,5-dihydropyrrole-3-carboxylate

To a stirred solution of Intermediate 49 in EtOH (9 mL) was added lithium hydroxide monohydrate (67 mg, 1.60 mmol) in water (3 mL). After 5 h, the reaction mixture was concentrated in vacuo, then freeze-dried from water/acetonitrile, to give the title compound (489 mg, quantitative) as a yellow solid. LCMS (Method 7): [M+H]⁺ m/z 612, RT 1.66 minutes.

### Intermediate 51

### tert-Butyl 3-{2-[(S)-benzyloxycarbonylamino(4,4-difluorocyclohexyl)methyl]imidazo-[1,2-b]pyridazin-7-yl}-4-(3,3,4,4-tetrafluoropyrrolidine-1-carbonyl)-2,5-dihydropyrrole-1-carboxylate

To a solution of Intermediate 50 (473 mg, 0.77 mmol) in DMF (2.60 mL) and DIPEA (0.53 mL, 3.0 mmol) was added 3,3,4,4-tetrafluoropyrrolidine hydrochloride (167 mg, 0.93 mmol), followed by HATU (328 mg, 0.85 mmol). The mixture was stirred for 2 h, then diluted with saturated aqueous sodium bicarbonate solution (30 mL) and DCM (30 mL). The organic layer was separated, and the aqueous layer was re-extracted with DCM (3×30 mL). The combined organic layers were dried and concentrated. The residue was purified by flash column chromatography, eluting with a gradient of 0-100% EtOAc in isohexane, then freeze-dried from water/acetonitrile, to afford the title compound (475 mg, 84%) as a pale yellow solid. LCMS (Method 7): [M+H]⁺ m/z 737, RT 2.72 minutes.

### Intermediate 52

### tert-Butyl (3RS,4RS)-3-{2-[(S)-amino(4,4-difluorocyclohexyl)methyl]imidazo[1,2-b]-pyridazin-7-yl}-4-(3,3,4,4-tetrafluoropyrrolidine-1-carbonyl)pyrrolidine-1-carboxylate

To a stirred solution of Intermediate 51 (280 mg, 0.38 mmol) in EtOH (4 mL) were added palladium hydroxide on carbon (30 mg, 0.043 mmol) and ammonium formate (481 mg, 7.63 mmol). The reaction mixture was thoroughly degassed, then placed under an atmosphere of hydrogen for 18 h. An additional portion of palladium hydroxide on carbon (26 mg) was added. The reaction mixture was degassed and replaced under an atmosphere of hydrogen for 36 h, then filtered through Celite® and concentrated in vacuo. The residue was dissolved in DCM (10 mL) and washed with 1.5M aqueous sodium carbonate solution (10 mL). The aqueous layer was re-extracted with DCM (10 mL). The combined organic extracts were passed down a phase separator, then concentrated in vacuo, to give the title compound (mixture of pyrrolidine cis isomers) (230 mg, quantitative) as a black foam. LCMS (Method 7): [M+H]⁺ m/z 605, RT 1.63 minutes.

### Intermediate 53 tert-Butyl (3RS,4RS)-3-(2-{(S)-(4,4-difluorocyclohexyl)[(4-methyl-1,2,5-oxadiazole-3-carbonyl)amino]methyl}imidazo[1,2-b]pyridazin-7-yl)-4-(3,3,4,4-tetrafluoropyrrolidine-1-carbonyl)pyrrolidine-1-carboxylate

To a solution of Intermediate 52 (230 mg, 0.38 mmol) in DMF (3.2 mL) and DIPEA (0.20 mL, 1.1 mmol) was added 4-methyl-1,2,5-oxadiazole-3-carboxylic acid (65 mg, 0.48 mmol), followed by HATU (189 mg, 0.48 mmol). The reaction mixture was stirred for 1.5 h, then diluted with water (25 mL) and extracted with EtOAc (3×25 mL). The combined organic extracts were passed down a phase separator and concentrated in vacuo. The residue was purified by flash column chromatography, eluting with a gradient of 0-100% EtOAc in isohexane, then freeze-dried from water/acetonitrile, to afford the title compound (mixture of pyrrolidine cis isomers) (171 mg, 44%) as an off-white solid. LCMS (Method 8): [M+H]⁺ m/z 715, RT 2.28 minutes.

### Intermediate 54

### N-[(S)-(4,4-Difluorocyclohexyl){7-[(3RS,4RS)-4-(3,3,4,4-tetrafluoropyrrolidine-1-carbonyl)pyrrolidin-3-yl]imidazo[1,2-b]pyridazin-2-yl}methyl]-4-methyl-1,2,5-oxadiazole-3-carboxamide

To a solution of Intermediate 53 (75 mg, 0.10 mmol) in DCM (2 mL) was added 4M HCl in 1,4-dioxane (2 mL). The reaction mixture was stirred for 1.5 h, then concentrated in vacuo. The residue was partitioned between EtOAc (20 mL) and saturated aqueous sodium bicarbonate solution (20 mL). The layers were separated, and the aqueous layer was re-extracted with EtOAc (2×20 mL). The combined organic extracts were dried and concentrated in vacuo, then freeze-dried from water/acetonitrile, to give the title compound (mixture of pyrrolidine cis isomers) (63.3 mg, 98%) as an off-white solid. LCMS (Method 7): [M+H]⁺ m/z 615, RT 2.07 minutes.

### Intermediate 55

### tert-Butyl 2-(6-aminopyridazin-4-yl)-4,4-difluoropiperidine-1-carboxylate

A solution of 5-chloropyridazin-3-amine (300 mg, 2.31 mmol), 1-(tert-butoxycarbonyl)-4,4-difluoropiperidine-2-carboxylic acid (920 mg, 3.47 mmol), {Ir[dF(CF₃)ppy]₂(dtbpy)}PF₆ (50.0 mg, 44.6 μmol), nickel(II) chloride ethylene glycol dimethyl ether complex (50.0 mg, 0.228 mmol), 4,4′-di-tert-butyl-2,2′-bipyridine (93.0 mg, 0.346 mmol) and caesium carbonate (1.13 g, 3.47 mmol) in DMF (25 mL) was purged with nitrogen gas for 5 minutes, then irradiated for 72 h under 450 nm LED. The solvent was removed under reduced pressure. The residue was purified by silica column chromatography, eluting with 0-20% MeOH in EtOAc, to afford the crude title compound (440 mg, 60%) as an orange solid, which was utilised without further purification. LCMS (Method 11): [M+H]⁺ m/z 315.2, RT 1.06 minutes.

### Intermediate 56

tert-Butyl 2-{2-[(S)-benzyloxycarbonylamino(4,4-difluorocyclohexyl)methyl]imidazo-[1,2-b]pyridazin-7-yl}-4,4-difluoropiperidine-1-carboxylate A solution of Intermediate 55 (430 mg, 1.37 mmol), benzyl N-[(1S)-3-bromo-1-(4,4-difluorocyclohexyl)-2-oxopropyl]carbamate (660 mg, 1.63 mmol) and sodium bicarbonate (140 mg, 1.66 mmol) in 2-propanol (15 mL) was stirred at 80° C. overnight. The reaction mixture was concentrated and the residue was purified by silica column chromatography, eluting with 0-40% EtOAc in hexanes, to afford the title compound (86 mg, 7%) as an orange solid. LCMS (Method 11): [M+H]⁺ m/z 620.2, RT 1.56 minutes.

### Intermediate 57

### tert-Butyl 2-(2-{(S)-(4,4-difluorocyclohexyl)[(4-methyl-1,2,5-oxadiazole-3-carbonyl)-amino]methyl}imidazo[1,2-b]pyridazin-7-yl)-4,4-difluoropiperidine-1-carboxylate

A mixture of Intermediate 56 (87.0 mg, 98 μmol), hydrogen chloride in 1,4-dioxane (4.0M, 70.0 μL, 0.28 mmol) and palladium on charcoal (10% w/w loading, 20.0 mg) in ethanol (5 mL) was stirred for 5 h at r.t. under a hydrogen atmosphere. The reaction mixture was filtered and washed with ethanol (5 mL). The filtrate was concentrated. The residue was combined with HATU (47.0 mg, 0.12 mmol), 4-methyl-1,2,5-oxadiazole-3-carboxylic acid (16.0 mg, 0.12 mmol) and DIPEA (0.10 mL, 0.60 mmol), and suspended in DMF (1.0 mL). The mixture was stirred for 2 h at r.t., then the solvent was removed under reduced pressure. The residue was purified by silica column chromatography, eluting with 0-40% EtOAc in hexanes, to afford the title compound (46 mg, 78%) as a yellow oil. LCMS (Method 11): [M+H]⁺ m/z 596.2, RT 1.52 minutes.

### Intermediate 58

### Ethyl 2-{2-[(S)-benzyloxycarbonylamino(4,4-difluorocyclohexyl)methyl]imidazo[1,2-b]-pyridazin-7-yl}-4,4-difluorobutanoate

To a solution of Intermediate 21 (2.80 g, 6.17 mmol) and triethylamine (2.58 mL, 18.5 mmol) in DCM (62 mL) was added N-(benzyloxycarbonyloxy)succinimide (1.90 g, 7.41 mmol). The reaction mixture was stirred at r.t. for 10 minutes, then diluted with DCM (40 mL) and washed with water (100 mL). The aqueous layer was extracted with DCM (2×50 mL), and the combined organic extracts were washed with brine (100 mL). The organic layer was passed through a phase separator and concentrated in vacuo. The crude material was purified by silica column chromatography, eluting with a gradient of 0-60% EtOAc in isohexane, to give the title compound (3.13 g, 92%) as an off-white amorphous solid. LCMS (Method 7): [M+H]⁺ m/z 551, RT 2.46 minutes.

### Intermediate 59

### 2-{2-[(S-Benzyloxycarbonylamino(4,4-difluorocyclohexyl)methyl]imidazo[1,2-b]-pyridazin-7-yl}-4,4-difluorobutanoic acid

To a solution of Intermediate 58 (1.20 g, 2.18 mmol) in THE (8.8 mL) and water (2.2 mL) was added lithium hydroxide monohydrate (186 mg, 4.36 mmol). The reaction mixture was warmed to 40° C. and stirred for 3.5 h, then neutralised with aqueous HCl (2M, 2.2 mL) and concentrated in vacuo, to give the title compound (1.32 g, 116%) as a yellow amorphous solid. LCMS (Method 7): [M+H]⁺ m/z 523, RT 1.39 minutes.

### Intermediate 60

### Benzyl N-{(S)-(4,4-difluorocyclohexyl)[7-(3,3-difluoro-1-{[(5-fluoropyridin-2-yl)-amino]carbamoyl}propyl)imidazo[1,2-b]pyridazin-2-yl]methyl}carbamate

To a solution of Intermediate 59 (1.14 g, 2.18 mmol) in EtOAc (21.8 mL) was added pyridine (0.88 mL, 10.9 mmol), followed by T3P® solution (1.68 mol/L in EtOAc, 3.24 mL, 5.45 mmol). The mixture was stirred for 5 minutes, then (5-fluoropyridin-2-yl)-hydrazine (339 mg, 2.62 mmol) was added. The reaction mixture was warmed to 40° C. and stirred for 5 minutes, then allowed to cool to r.t. A few chips of ice were added, and the mixture was stirred for a further 5 minutes. EtOAc (50 mL) was added, and the organic layer was washed with water (50 mL). The aqueous layer was extracted with EtOAc (2×50 mL). The combined organic extracts were passed through a phase separator and concentrated in vacuo. The crude material was purified by silica column chromatography, eluting with a gradient of 0-100% EtOAc in isohexane, to give the title compound (755 mg, 55%) as an orange amorphous solid. LCMS (Method 7): [M+H]⁺ m/z 632, RT 2.21 minutes.

### Intermediate 61

### Benzyl N-[(S)-(4,4-difluorocyclohexyl){7-[3,3-difluoro-1-(6-fluoro-[1,2,4]triazolo[4,3-a]-pyridin-3-yl)propyl]imidazo[1,2-b]pyridazin-2-yl}methyl]carbamate

To a solution of Intermediate 60 (750 mg, 1.19 mmol) in THE (3 mL) at 0° C. was added triethylamine (0.66 mL, 4.75 mmol), followed by triphenylphosphine (623 mg, 2.38 mmol) and hexachloroethane (568 mg, 2.38 mmol). The reaction mixture was warmed to 40° C. and stirred for 1.5 h, then diluted with EtOAc (40 mL) and washed with water (40 mL). The aqueous layer was extracted with EtOAc (2×20 mL). The combined organic layers were passed through a phase separator and concentrated in vacuo. The crude material was purified by silica column chromatography, eluting with a gradient of 0-100% EtOAc in isohexane, to give the title compound (706 mg, 97%) as an off-white amorphous solid. LCMS (Method 7): [M+H]⁺ m/z 614, RT 2.11 minutes.

### Intermediate 62

### (S)-(4,4-Difluorocyclohexyl){7-[3,3-difluoro-1-(6-fluoro-[1,2,4]triazolo[4,3-a]pyridin-3-yl)propyl]imidazo[1,2-b]pyridazin-2-yl}methanamine

To a solution of Intermediate 61 (700 mg, 1.14 mmol) in DCM (11.4 mL) at 0° C. was added boron tribromide solution (1.0M in DCM) (2.28 mL, 2.28 mmol) dropwise. The reaction mixture was allowed to warm to r.t. and stirred for 1.5 h, then quenched with saturated aqueous sodium bicarbonate solution (20 mL). The aqueous layer was extracted with DCM (3×20 mL). The combined organic extracts were passed through a phase separator and concentrated in vacuo. The crude material was purified by silica column chromatography, eluting with a gradient of 0-20% MeOH in DCM, to give the title compound (361 mg, 66%) as a colourless amorphous solid. LCMS (Method 7): [M+H]⁺ m/z 480, RT 1.41 minutes.

### Intermediate 63

### tert-Butyl N-(5-chloropyridazin-3-yl)carbamate

To a nitrogen degassed mixture of 3,5-dichloropyridazine (60.00 g, 0.403 mol), tert-butyl carbamate (48.63 g, 0.415 mol), cesium carbonate (266 g, 0.819 mol) and XantPhos (23.51 g, 40.6 mmol) in dry 1,4-dioxane (0.6 L) was added palladium(II) acetate (4.60 g, 20.5 mmol). The reaction mixture was further degassed, then heated at 80° C. for 18 h. The cooled reaction mixture was filtered through Celite®, rinsing through with 1,4-dioxane (4×150 mL). The filtrate was concentrated in vacuo, then DCM (0.25 L), water (0.25 L) and brine (0.25 L) were added. The two-phase system was filtered through glass-fibre paper, and the organic layer was retained. The aqueous layer was re-extracted with DCM (100 mL). The organic layers were combined and dried over magnesium sulphate, then filtered and concentrated in vacuo. The residue was dissolved in DCM (100 mL) and purified by dry flash chromatography, eluting with 0-30% EtOAc in heptane, to afford the title compound (37.8 g, 41%) as an off-white solid. LCMS (Method 6): [M+H-56]⁺ m/z 174.0/176.0, RT 2.35 minutes.

### Intermediate 64

### Methyl 4-[6-(tert-butoxvcarbonylamino)pyridazin-4-yl]tetrahydropyran-4-carboxylate

A solution of methyl tetrahydropyran-4-carboxylate (0.51 mL, 3.8 mmol) and Intermediate 63 (0.44 g, 1.92 mmol) in THE (10 mL) was added dropwise to lithium bis(trimethylsilyl)amide (1.0M in toluene) (5.8 mL, 5.8 mmol) under nitrogen at 0° C. The reaction mixture was stirred at 0° C. for 3 h, then at r.t. for a further 2 h, then quenched by the addition of saturated aqueous NH₄Cl solution (50 mL) and DCM (40 mL). The layers were separated, and the aqueous layer was extracted with DCM (2×20 mL). The combined organic layers were passed through a phase separator and concentrated in vacuo. The crude residue was purified by flash column chromatography, eluting with a gradient of 0-100% EtOAc in hexanes, to give the title compound (350 mg, 54%) as a white solid. LCMS (Method 11): [M+H]⁺ m/z 338.0, RT 1.06 minutes.

### Intermediate 65

### Methyl 4-(6-aminopyridazin-4-yl)tetrahydropyran-4-carboxylate

To a solution of Intermediate 64 (341 mg, 1.01 mmol) in DCM (5 mL) was added TFA (2.5 mL). The reaction mixture was stirred at r.t. for 1 h, then concentrated in vacuo and diluted with DCM (20 mL). The organic layer was washed with saturated aqueous sodium bicarbonate solution (20 mL), and the aqueous layer was back-extracted with a mixture of MeOH in DCM (2%, 2×20 mL). The combined organic extracts were passed through a phase separator, then concentrated in vacuo, to give the title compound (250 mg) as a colourless amorphous solid, which was utilised without further purification. LCMS (Method 7): [M+H]⁺ m/z 238.2, RT 0.43 minutes.

### Intermediate 66

### Methyl 4-{2-[(S)-benzyloxycarbonylamino(4,4-difluorocyclohexyl)methyl]imidazo[1,2-b]pyridazin-7-yl}tetrahydropyran-4-carboxylate

To a mixture of Intermediate 65 (240 mg, 1.01 mmol), benzyl N-[(1S)-3-bromo-1-(4,4-difluorocyclohexyl)-2-oxopropyl]carbamate (613 mg, 1.52 mmol) and sodium bicarbonate (170 mg, 2.02 mmol) was added 2-propanol (5 mL). The reaction mixture was stirred at 80° C. overnight, then concentrated in vacuo. The residue was dissolved in EtOAc (20 mL) and washed with water (20 mL). The aqueous layer was extracted with EtOAc (2×20 mL). The combined organic extracts were passed through a phase separator and concentrated in vacuo. The crude material was purified by column chromatography (Biotage SFAR HC DUO, 25 g, Isolera), eluting with a gradient of 0-80% EtOAc in isohexane, to give the title compound (304 mg, 53%) as an orange solid. LCMS (Method 7): [M+H]⁺ m/z 543.2, RT 2.17 minutes.

### Intermediate 67

### Lithium 4-{2-[(S)-benzyloxycarbonylamino(4,4-difluorocyclohexyl)methyl]imidazo[1,2-b]pyridazin-7-yl}tetrahydropyran-4-carboxylate

Intermediate 66 (300 mg, 0.53 mmol) was dissolved in THF (2 mL) and treated with lithium hydroxide monohydrate (53 mg, 1.24 mmol) dissolved in water (1 mL). MeOH (1 mL) was added to aid dissolution. The mixture was stirred overnight, then evaporated in vacuo, to afford the title compound (304 mg, 92%) as a white solid, which was utilised without further purification. LCMS (Method 7): [M+H]⁺ m/z 529.2, RT 1.32 minutes.

### Intermediate 68

### Benzyl N-[(S)-(4,4-difluorocyclohexyl){7-[4-(2,2-difluoropropylcarbamoyl)-tetrahydropyran-4-yl]imidazo[1,2-b]pyridazin-2-yl}methyl]carbamate

Intermediate 67 (302 mg, 0.535 mmol) and 2,2-difluoropropylamine hydrochloride (82 mg, 0.59 mmol) were suspended in DCM (10 mL) and treated with DIPEA (3 mL, 17.2 mmol). The mixture was stirred for 1 minute, then HATU (218 mg, 0.562 mmol) was added. DMF (1 mL) was added to aid dissolution. The mixture was left stirring overnight, then separated between EtOAc (50 mL) and brine (50 mL). The organic layers were washed with brine (3×20 mL), then passed through a hydrophobic frit and evaporated in vacuo. The brown gum was purified by silica column chromatography, eluting with a 0-100% gradient of EtOAc in DCM, to give the title compound (250 mg, 73%). LCMS (Method 11): [M+H]⁺ m/z 606.2, RT 1.12 minutes.

### Intermediate 69

### 4-{2-[(S)-Amino(4,4-difluorocyclohexyl)methyl]imidazo[1,2-b]pyridazin-7-yl}-N-(2,2-difluoropropyl)tetrahydropyran-4-carboxamide

Intermediate 68 (245 mg, 0.397 mmol) was dissolved in ethanol (10 mL) and degassed with nitrogen. Palladium on carbon (50 mg, 0.047 mmol) was added. The reaction mixture was placed under a hydrogen gas atmosphere and stirred at r.t. for 24 h, then filtered through Celite®, washed with DCM and evaporated in vacuo, to give the title compound (200 mg, 97%) as a pale grey solid. LCMS (Method 11): [M+H]⁺ m/z 472.2, RT 0.83 minutes.

### Example 1

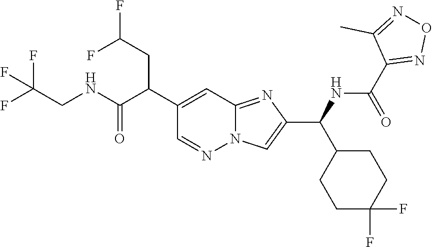 [SMILES: Cc1nonc1C(=O)N[C@H](c1cn2ncc(C(CC(F)F)C(=O)NCC(F)(F)F)cc2n1)C1CCC(F)(F)CC1]

### N-[(S)-(4,4-Difluorocyclohexyl){7-[3,3-difluoro-1-(2,2,2-trifluoroethylcarbamoyl)-propyl]imidazo[1,2-b]pyridazin-2-yl}methyl]-4-methyl-1,2,5-oxadiazole-3-carboxamide

To a solution of 2-chloro-1-methylpyridinium iodide (21 mg, 0.0809 mmol) in DMA (0.2 mL) were added DIPEA (0.055 mL, 0.311 mmol) and 2,2,2-trifluoroethanamine (0.0074 mL, 0.0933 mmol). Intermediate 13 (31 mg, 0.0622 mmol) in DMA was added. The reaction mixture was heated at 50° ° C. for 18 h, then cooled and diluted with EtOAc (10 mL), water (10 mL) and brine (10 mL). The organic layer was dried over magnesium sulfate, and the solvent was removed under vacuum. The resulting brown oil was purified by silica column chromatography, eluting with 0-50% EtOAc in heptane, to afford the title compound (19 mg, 53%) as a light brown gum. δ_{H} (400 MHz, CD₃OD) 8.47 (d, J 2.1 Hz, 1H), 8.14 (s, 1H), 7.91 (d, J 2.0 Hz, 1H), 5.75-6.13 (m, 1H), 5.26 (d, J 8.6 Hz, 1H), 3.93-4.00 (m, 1H), 3.90 (td, J 9.4, 2.0 Hz, 2H), 2.67-2.87 (m, 1H), 2.52 (s, 3H), 2.29-2.47 (m, 1H), 2.23 (d, J 8.6 Hz, 1H), 2.10 (d, J 48.8 Hz, 2H), 1.58-1.91 (m, 3H), 1.24-1.58 (m, 3H). LCMS (Method 3): [M+H]⁺ m/z 580, RT 3.47 minutes.

### Example 2

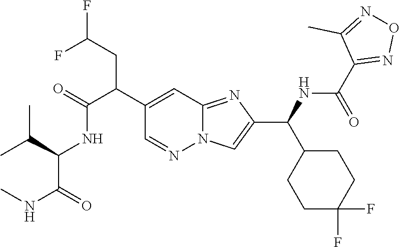 [SMILES: CNC(=O)[C@H](NC(=O)C(CC(F)F)c1cnn2cc([C@@H](NC(=O)c3nonc3C)C3CCC(F)(F)CC3)nc2c1)C(C)C]

### N-{(S)-(4,4-difluorocyclohexyl)[7-(3,3-difluoro-1-{[(1R)-2-methyl-1-(methylcarbamoyl)-propyl]carbamoyl}propyl)imidazo[1,2-b]pyridazin-2-yl]methyl}-4-methyl-1,2,5-oxadiazole-3-carboxamide

To a solution of Intermediate 13 (40 mg, 0.0803 mmol) in DMF (1 mL) were added DIPEA (70 μL, 0.401 mmol), Intermediate 15 (89 mg, 0.241 mmol) and HATU (37 mg, 0.0963 mmol). The reaction mixture was stirred for 18 h, then diluted with water (10 mL) and extracted with EtOAc (25 mL). The organic layer was washed with water (3×10 mL) and dried over magnesium sulfate, then the solvent was removed. The resulting oil was purified by silica column chromatography, eluting with 0-70% EtOAc in heptane, to afford the title compound (30 mg, 61%) as a white solid. δ_{H} (400 MHz, CD₃OD) 8.49 (dd, J 15.5, 2.1 Hz, 1H), 8.13 (d, J 10.6 Hz, 1H), 7.92 (dd, J 18.9, 2.0 Hz, 1H), 5.94 (dddd, J 56.3, 51.8, 9.4, 4.8 Hz, 1H), 5.25 (dd, J 8.6, 3.4 Hz, 1H), 4.00-4.14 (m, 2H), 2.66-2.84 (m, 3H), 2.61 (s, 1H), 2.52 (s, 2H), 2.28-2.43 (m, 1H), 2.22 (d, J 9.2 Hz, 1H), 1.93-2.13 (m, 3H), 1.59-1.90 (m, 4H), 1.30-1.57 (m, 3H), 0.96 (dd, J 21.9, 6.8 Hz, 3H), 0.78 (dd, J 18.8, 6.8 Hz, 3H). LCMS (Method 2): [M+H]⁺ m/z 611, RT 3.18 minutes.

### Examples 3A & 3B

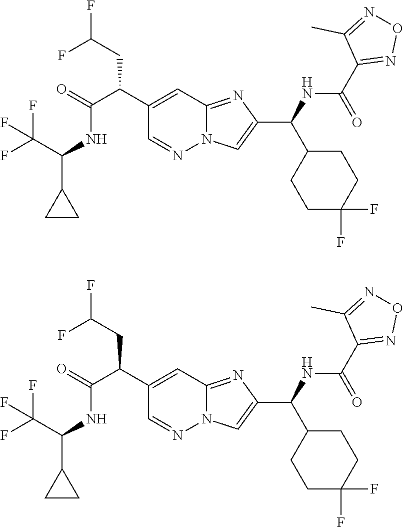 [SMILES: Cc1nonc1C(=O)N[C@H](c1cn2ncc([C@@H](CC(F)F)C(=O)N[C@@H](C3CC3)C(F)(F)F)cc2n1)C1CCC(F)(F)CC1 ; Cc1nonc1C(=O)N[C@H](c1cn2ncc([C@H](CC(F)F)C(=O)N[C@@H](C3CC3)C(F)(F)F)cc2n1)C1CCC(F)(F)CC1]

### N-[(S)-{7-[(1R)-1-{[(1S)-1-Cyclopropyl-2,2,2-trifluoroethyl]carbamoyl}-3,3-difluoro-propyl]imidazo[1,2-b]pyridazin-2-yl}(4,4-difluorocyclohexyl)methyl]-4-methyl-1,2,5-oxadiazole-3-carboxamide

### N-[(S)-{7-[(1S)-1-{[(1S)-1-Cyclopropyl-2,2,2-trifluoroethyl]carbamoyl}-3,3-difluoro-propyl]imidazo[1,2-b]pyridazin-2-yl}(4,4-difluorocyclohexyl)methyl]-4-methyl-1,2,5-oxadiazole-3-carboxamide

Intermediate 13 (94 mg, 0.188 mmol) in DMA (1 mL) was added to a solution of 2-chloro-1-methylpyridinium iodide (63 mg, 0.244 mmol), (1S)-1-cyclopropyl-2,2,2-trifluoroethanamine (31 mg, 0.225 mmol) and DIPEA (164 μL, 0.939 mmol) in DMA (1 mL). The reaction mixture was heated at 50° C. in a sealed tube for 1 h, then diluted with water (10 mL) and extracted with ethyl acetate (2×10 mL). The organic extracts were dried with sodium sulfate and concentrated under vacuum. The residue was purified by silica column chromatography, eluting with a gradient of 0-100% ethyl acetate in heptane, then reverse-phase column chromatography, eluting with 5-100% acetonitrile in water with 0.1% formic acid, then preparative HPLC (Method 9). The stereoisomers were separated by chiral HPLC (Chiralcel OD-H, 20×250 mm, 5 μm, eluting with 95% heptane and 5% IPA at 18 mL/minute) to afford the title compounds (Peak 1, 13 mg, 11%; and Peak 2, 22 mg, 19%) as white solids.

Peak 1: δ_{H} (400 MHz, DMSO-d₆) 9.42 (d, J 9.0 Hz, 1H), 8.95 (d, J 9.1 Hz, 1H), 8.46 (d, J 2.1 Hz, 1H), 8.25 (s, 1H), 7.92 (d, J 2.0 Hz, 1H), 6.09 (tt, J 56.2, 4.3 Hz, 1H), 5.17 (t, J 8.6 Hz, 1H), 4.07-3.87 (m, 2H), 2.83-2.61 (m, 1H), 2.46 (s, 3H), 2.44-2.28 (m, 1H), 2.23-2.11 (m, 1H), 2.11-1.94 (m, 2H), 1.94-1.67 (m, 3H), 1.67-1.54 (m, 1H), 1.47-1.32 (m, 1H), 1.32-1.19 (m, 1H), 1.15-1.00 (m, 1H), 0.68-0.59 (m, 1H), 0.59-0.46 (m, 2H), 0.36-0.23 (m, 1H). LCMS (Method 3): [M+H]⁺ m/z 620.3, RT 3.70 minutes. Chiral LC (Chiralcel OD-H, 4.6×250 mm, 5 μm, eluting with 95% heptane and 5% IPA at 1 mL/minute, 45 minutes) RT 21.55 minutes.
Peak 2: δ_{H} (400 MHz, DMSO-d₆) 9.43 (d, J 9.0 Hz, 1H), 8.94 (d, J 9.1 Hz, 1H), 8.50 (d, J 2.1 Hz, 1H), 8.26 (s, 1H), 7.97 (d, J 2.0 Hz, 1H), 6.04 (tt, J 56.1, 4.3 Hz, 1H), 5.18 (t, J 8.6 Hz, 1H), 4.09-3.91 (m, 2H), 2.79-2.59 (m, 1H), 2.46 (s, 3H), 2.44-2.34 (m, 1H), 2.25-2.12 (m, 1H), 2.12-1.94 (m, 2H), 1.94-1.66 (m, 3H), 1.66-1.54 (m, 1H), 1.46-1.33 (m, 1H), 1.33-1.19 (m, 1H), 1.06-0.93 (m, 1H), 0.56-0.42 (m, 1H), 0.35-0.18 (m, 2H), 0.11-0.00 (m, 1H). LCMS (Method 3): [M+H]⁺ m/z 620.3, RT 3.69 minutes. Chiral LC (Chiralcel OD-H, 4.6×250 mm, 5 μm, eluting with 95% heptane and 5% IPA at 1 mL/minute, 45 minutes) RT 32.88 minutes.

### General Method 1

Intermediate 11 (72 mg, 0.153 mmol) and the specified carboxylic acid derivative (0.169 mmol) were stirred in DMF (1 mL). DIPEA (0.055 mL, 0.307 mmol) was added, followed by HATU (70 mg, 0.184 mmol). The reaction mixture was stirred for 40 minutes, then diluted with ethyl acetate (3 mL). The resulting material was washed with water (2 mL), saturated aqueous NH₄Cl solution (2 mL), water (2 mL) and brine (2 mL), then concentrated under vacuum. The residue was purified by reverse-phase column chromatography, eluting with a gradient of 5-100% acetonitrile in water with 0.1% formic acid, followed by preparative HPLC (Method 9).

### Examples 4a & 4B

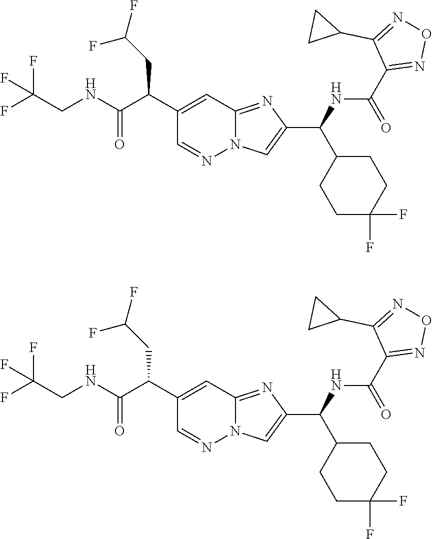 [SMILES: O=C(N[C@H](c1cn2ncc([C@H](CC(F)F)C(=O)NCC(F)(F)F)cc2n1)C1CCC(F)(F)CC1)c1nonc1C1CC1 ; O=C(N[C@H](c1cn2ncc([C@@H](CC(F)F)C(=O)NCC(F)(F)F)cc2n1)C1CCC(F)(F)CC1)c1nonc1C1CC1]

### 4-Cyclopropyl-N-[(S)-(4,4-difluorocyclohexyl){7-[(1R)-3,3-difluoro-1-(2,2,2-trifluoro-ethylcarbamoyl)propyl]imidazo[1,2-b]pyridazin-2-yl}methyl]-1,2,5-oxadiazole-3-carboxamide

### 4-Cyclopropyl-N-[(S)-(4,4-difluorocyclohexyl){7-[(1R)-3,3-difluoro-1-(2,2,2-trifluoro-ethylcarbamoyl)propyl]imidazo[1,2-b]pyridazin-2-yl}methyl]-1,2,5-oxadiazole-3-carboxamide

Prepared from 4-cyclopropyl-1,2,5-oxadiazole-3-carboxylic acid (26 mg, 0.169 mmol) in accordance with General Method 1, followed by separation of the stereoisomers by chiral SFC (Chiralcel OJ-H, 10×250 mm, 5 μm, eluting with 5% MeOH and 95% CO₂ at 15 mL/minute), to afford the title compounds (Peak 1, 10 mg, 11%; and Peak 2, 10 mg, 11%) as white solids.

Peak 1: δ_{H} (500 MHz, DMSO-d₆) 9.50 (d, J 9.0 Hz, 1H), 8.94 (t, J 6.3 Hz, 1H), 8.49 (d, J 2.1 Hz, 1H), 8.25 (s, 1H), 7.96 (d, J 2.0 Hz, 1H), 6.06 (tt, J 56.2, 4.4 Hz, 1H), 5.19 (t, J 8.5 Hz, 1H), 4.06-3.79 (m, 3H), 2.79-2.65 (m, 1H), 2.48-2.37 (m, 1H), 2.31-2.24 (m, 1H), 2.23-2.13 (m, 1H), 2.11-1.94 (m, 2H), 1.89 (d, J 11.5 Hz, 1H), 1.86-1.69 (m, 2H), 1.66-1.51 (m, 1H), 1.46-1.33 (m, 1H), 1.33-1.21 (m, 1H), 1.17-1.06 (m, 2H), 1.00-0.91 (m, 2H). LCMS (Method 3): [M+H]⁺ m/z 606.3, RT 3.63 minutes. Chiral SFC (Cellulose-3, 4.6×250 mm, 5 μm, eluting with 5% MeOH and 95% CO₂ at 4 mL/minute, 18 minutes) RT 6.40 minutes.
Peak 2: δ_{H} (500 MHz, DMSO-d₆) 9.50 (d, J 9.0 Hz, 1H), 8.95 (t, J 6.3 Hz, 1H), 8.49 (d, J 2.1 Hz, 1H), 8.25 (s, 1H), 7.96 (d, J 2.0 Hz, 1H), 6.06 (tt, J 56.1, 4.3 Hz, 1H), 5.19 (t, J 8.5 Hz, 1H), 4.03-3.81 (m, 3H), 2.77-2.65 (m, 1H), 2.48-2.38 (m, 1H), 2.31-2.23 (m, 1H), 2.23-2.12 (m, 1H), 2.10-1.94 (m, 2H), 1.94-1.86 (m, 1H), 1.86-1.69 (m, 2H), 1.65-1.56 (m, 1H), 1.44-1.33 (m, 1H), 1.33-1.21 (m, 1H), 1.15-1.07 (m, 2H), 0.99-0.91 (m, 2H). LCMS (Method 3): [M+H]⁺ m/z 606.3, RT 3.63 minutes. Chiral SFC (Cellulose-3, 4.6×250 mm, 5 μm, eluting with 5% MeOH and 95% CO₂ at 4 mL/minute, 18 minutes) RT 11.16 minutes.

### Example 5

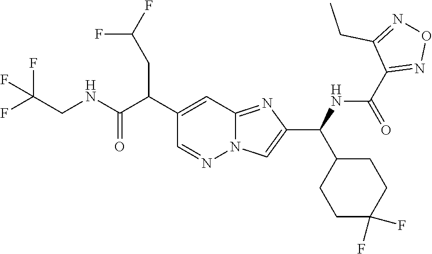 [SMILES: CCc1nonc1C(=O)N[C@H](c1cn2ncc(C(CC(F)F)C(=O)NCC(F)(F)F)cc2n1)C1CCC(F)(F)CC1]

### N-[(S)-(4,4-Difluorocyclohexyl){7-[3,3-difluoro-1-(2,2,2-trifluoroethylcarbamoyl)-propyl]imidazo[1,2-b]pyridazin-2-yl}methyl]-4-ethyl-1,2,5-oxadiazole-3-carboxamide

Prepared from 4-ethyl-1,2,5-oxadiazole-3-carboxylic acid (24 mg, 0.169 mmol) in accordance with General Method 1 to afford the title compound (50 mg, 54%) as a white solid. δ_{H} (400 MHz, DMSO-d₆) 9.45 (dd, J 9.0, 1.5 Hz, 1H), 8.94 (t, J 6.2 Hz, 1H), 8.48 (d, J 2.1 Hz, 1H), 8.24 (s, 1H), 7.95 (d, J 1.9 Hz, 1H), 6.06 (tt, J 56.1, 4.3 Hz, 1H), 5.17 (t, J 8.6 Hz, 1H), 4.05-3.81 (m, 3H), 2.94-2.84 (m, 2H), 2.80-2.61 (m, 1H), 2.45-2.36 (m, 1H), 2.24-2.12 (m, 1H), 2.11-1.94 (m, 2H), 1.94-1.67 (m, 3H), 1.67-1.55 (m, 1H), 1.47-1.32 (m, 1H), 1.32-1.15 (m, 4H). LCMS (Method 3): [M+H]⁺ m/z 594.2, RT 3.58 minutes.

### Examples 6A & 6B

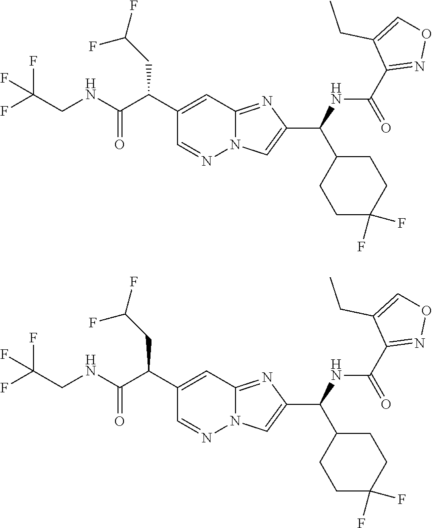

### N-[(S)-(4,4-Difluorocyclohexyl){7-[(1R)-3,3-difluoro-1-(2,2,2-trifluoroethylcarbamoyl)-propyl]imidazo[1,2-b]pyridazin-2-yl}methyl]-4-ethylisoxazole-3-carboxamide N-[(S)-(4,4-Difluorocyclohexyl){7-[(1S)-3,3-difluoro-1-(2,2,2-trifluoroethylcarbamoyl)-propyl]imidazo[1,2-b]pyridazin-2-yl}methyl]-4-ethylisoxazole-3-carboxamide

Prepared from 4-ethylisoxazole-3-carboxylic acid (24 mg, 0.169 mmol) in accordance with General Method 1, followed by separation of the stereoisomers by chiral SFC (Chiralcel OJ-H, 10×250 mm, 5 μm, eluting with 5% IPA and 95% CO₂ at 15 mL/minute) to afford the title compounds (Peak 1, 10 mg, 11%; and Peak 2, 8 mg, 9%) as white solids.

Peak 1: δ_{H} (400 MHz, DMSO-d₆) 9.38 (s, 1H), 8.93 (t, J 6.4 Hz, 1H), 8.68 (d, J 9.0 Hz, 1H), 8.47 (d, J 2.1 Hz, 1H), 8.22 (s, 1H), 7.93 (d, J 2.0 Hz, 1H), 6.06 (tt, J 56.3, 4.4 Hz, 1H), 5.16 (t, J 8.5 Hz, 1H), 4.04-3.77 (m, 3H), 2.90-2.77 (m, 2H), 2.75-2.61 (m, 1H), 2.46-2.34 (m, 1H), 2.20-2.09 (m, 1H), 2.07-1.92 (m, 2H), 1.89-1.67 (m, 3H), 1.65-1.55 (m, 1H), 1.45-1.32 (m, 1H), 1.32-1.19 (m, 1H), 1.15 (t, J 7.5 Hz, 3H). LCMS (Method 3): [M+H]⁺ m/z 593.3, RT 3.23 minutes. Chiral SFC (Chiralpak IC, 4.6×250 mm, 5 μm, eluting with 10% IPA and 90% CO₂ at 4 mL/minute, 6 minutes) RT 2.88 minutes.
Peak 2: δ_{H} (400 MHz, DMSO-d₆) 9.38 (s, 1H), 9.00-8.91 (m, 1H), 8.70 (d, J 9.0 Hz, 1H), 8.47 (d, J 2.1 Hz, 1H), 8.23 (s, 1H), 7.93 (d, J 1.9 Hz, 1H), 6.06 (tt, J 56.2, 4.4 Hz, 1H), 5.16 (t, J 8.5 Hz, 1H), 4.06-3.79 (m, 3H), 2.89-2.78 (m, 2H), 2.78-2.62 (m, 1H), 2.47-2.35 (m, 1H), 2.21-2.09 (m, 1H), 2.08-1.90 (m, 2H), 1.90-1.66 (m, 3H), 1.66-1.53 (m, 1H), 1.45-1.32 (m, 1H), 1.32-1.19 (m, 1H), 1.15 (t, J 7.5 Hz, 3H). LCMS (Method 3): [M+H]+m/z 593.3, RT 3.24 minutes. Chiral SFC (Chiralpak IC, 4.6×250 mm, 5 μm, eluting with 10% IPA and 90% CO₂ at 4 mL/minute, 6 minutes) RT 4.15 minutes.

### Example 7

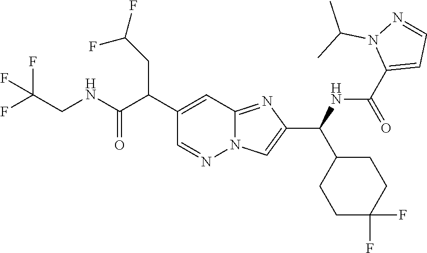 [SMILES: CC(C)n1nccc1C(=O)N[C@H](c1cn2ncc(C(CC(F)F)C(=O)NCC(F)(F)F)cc2n1)C1CCC(F)(F)CC1]

### N-[(S)-(4,4-Difluorocyclohexyl){7-[3,3-difluoro-1-(2,2,2-trifluoroethylcarbamoyl)-propyl]imidazo[1,2-b]pyridazin-2-yl}methyl]-2-isopropylpyrazole-3-carboxamide

Prepared from 1-isopropyl-1H-pyrazole-5-carboxylic acid (26 mg, 0.169 mmol) in accordance with General Method 1 to afford the title compound (53 mg, 56%) as a white solid. δ_{H} (400 MHz, DMSO-d₆) 8.93 (t, J 5.8 Hz, 1H), 8.72 (d, J 9.0 Hz, 1H), 8.47 (d, J 2.1 Hz, 1H), 8.22 (s, 1H), 7.94 (d, J 2.0 Hz, 1H), 7.48 (d, J 1.8 Hz, 1H), 6.90 (t, J 2.2 Hz, 1H), 6.06 (tt, J 56.1, 4.3 Hz, 1H), 5.43-5.27 (m, 1H), 5.15 (t, J 8.7 Hz, 1H), 4.07-3.77 (m, 3H), 2.81-2.60 (m, 1H), 2.44-2.35 (m, 1H), 2.27-2.11 (m, 1H), 2.11-1.92 (m, 2H), 1.92-1.65 (m, 3H), 1.65-1.54 (m, 1H), 1.44-1.16 (m, 8H). LCMS (Method 3): [M+H]⁺ m/z 606.3, RT 3.29 minutes.

### Example 8

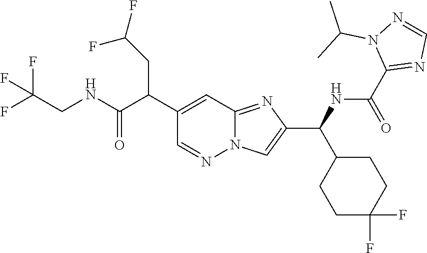 [SMILES: CC(C)n1ncnc1C(=O)N[C@H](c1cn2ncc(C(CC(F)F)C(=O)NCC(F)(F)F)cc2n1)C1CCC(F)(F)CC1]

### N-[(S)-(4,4-Difluorocyclohexyl){7-[3,3-difluoro-1-(2,2,2-trifluoroethylcarbamoyl)-propyl]imidazo[1,2-b]pyridazin-2-yl}methyl]-2-isopropyl-1,2,4-triazole-3-carboxamide

Prepared from 2-isopropyl-1,2,4-triazole-3-carboxylic acid (26 mg, 0.169 mmol) in accordance with General Method 1 to afford the title compound (48 mg, 50%) as a white solid. δ_{H} (400 MHz, DMSO-d₆) 8.97-8.91 (m, 1H), 8.89 (dd, J 9.2, 1.3 Hz, 1H), 8.48 (d, J 2.1 Hz, 1H), 8.29 (s, 1H), 8.09 (s, 1H), 7.98 (t, J 2.1 Hz, 1H), 6.06 (tt, J 56.1, 4.4 Hz, 1H), 5.61-5.43 (m, 1H), 5.14 (t, J 8.6 Hz, 1H), 4.06-3.78 (m, 3H), 2.79-2.60 (m, 1H), 2.46-2.36 (m, 1H), 2.22-2.10 (m, 1H), 2.09-1.93 (m, 2H), 1.93-1.84 (m, 1H), 1.84-1.65 (m, 2H), 1.65-1.51 (m, 1H), 1.45-1.17 (m, 8H). LCMS (Method 3): [M+H]*m/z 607.3, RT 3.43 minutes.

### Example 9

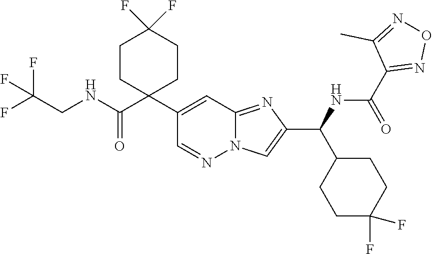 [SMILES: Cc1nonc1C(=O)N[C@H](c1cn2ncc(C3(C(=O)NCC(F)(F)F)CCC(F)(F)CC3)cc2n1)C1CCC(F)(F)CC1]

### N-[(S)-(4,4-Difluorocyclohexyl){7-[4,4-difluoro-1-(2,2,2-trifluoroethylcarbamoyl)-cyclohexyl]imidazo[1,2-b]pyridazin-2-yl}methyl]-4-methyl-1,2,5-oxadiazole-3-carboxamide

HATU (131 mg, 0.34 mmol) was added to a stirred solution of Intermediate 28 (92%, 150 mg, 0.26 mmol), 2,2,2-trifluoroethanamine (42 μL, 0.53 mmol) and DIPEA (92 μL, 0.53 mmol) in anhydrous DMF (2.6 mL). The reaction mixture was stirred at 20° C. under nitrogen for 18 h, then quenched with saturated aqueous sodium hydrogen carbonate solution (10 mL) and diluted with water (10 mL). The resulting material was extracted with EtOAc (3×30 mL). The combined organic extracts were washed successively with 0.5M aqueous hydrochloric acid/brine (1:1, 20 mL) and water/brine (1:1, 3×20 mL). The organic phase was dried over magnesium sulfate, then filtered and concentrated in vacuo. The residue was purified by basic preparative HPLC to afford the title compound (98% purity) (118 mg, 73%) as a white powder. δ_{H} (400 MHz, DMSO-d₆) 9.41 (d, J 8.9 Hz, 1H), 8.47 (t, J 5.9 Hz, 1H), 8.40 (d, J 2.3 Hz, 1H), 8.25 (s, 1H), 8.00 (d, J 2.0 Hz, 1H), 5.17 (t, J 8.4 Hz, 1H), 3.87 (qd, ³J_{HF} 9.7, J 6.2 Hz, 2H), 2.63-2.54 (m, 2H), 2.47 (s, 3H), 2.25-2.12 (m, 1H), 2.12-1.67 (m, 11H), 1.66-1.55 (m, 1H), 1.47-1.20 (m, 2H). LCMS (Method 3): [M+H]⁺ 620, RT 3.72 minutes.

### Examples 10A & 10B

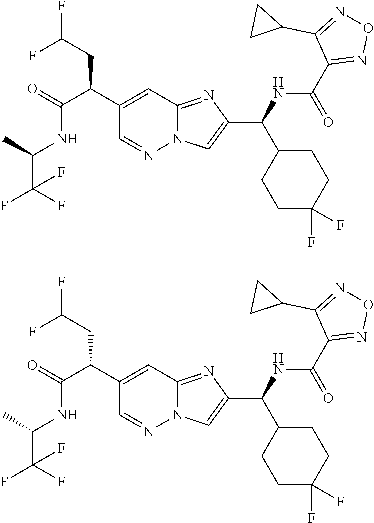 [SMILES: C[C@@H](NC(=O)[C@@H](CC(F)F)c1cnn2cc([C@@H](NC(=O)c3nonc3C3CC3)C3CCC(F)(F)CC3)nc2c1)C(F)(F)F ; C[C@H](NC(=O)[C@H](CC(F)F)c1cnn2cc([C@@H](NC(=O)c3nonc3C3CC3)C3CCC(F)(F)CC3)nc2c1)C(F)(F)F]

### 4-Cyclopropyl-N-[(S)-(4,4-difluorocyclohexyl){7-[(1S)-3,3-difluoro-1-{[(1S)-2,2,2-trifluoro-1-methylethyl]carbamoyl}propyl]imidazo[1,2-b]pyridazin-2-yl}methyl]-1,2,5-oxadiazole-3-carboxamide

### 4-Cyclopropyl-N-[(S)-(4,4-difluorocyclohexyl){7-[(1R)-3,3-difluoro-1-{[(1S)-2,2,2-trifluoro-1-methylethyl]carbamoyl}propyl]imidazo[1,2-b]pyridazin-2-yl}methyl]-1,2,5-oxadiazole-3-carboxamide

To a solution of Intermediate 34 (109 mg, 0.226 mmol), 4-cyclopropyl-1,2,5-oxadiazole-3-carboxylic acid (35 mg, 0.23 mmol) and DIPEA (88 mg, 0.68 mmol) in DMF (2.3 mL) was added HATU (106 mg, 0.270 mmol). The reaction mixture was stirred at r.t. for 5 minutes, then diluted with DCM (20 mL) and washed with water (20 mL). The aqueous layer was extracted with DCM (2×20 mL), and the combined organic extracts were passed through a phase separator and concentrated in vacuo. The crude material was purified by silica column chromatography, eluting with a gradient of 0-80% EtOAc in isohexane. The resulting diastereomeric mixture (1:1) was subject to chiral SFC purification (Chiralpak IC 250×20 mm, 5 μm column, flow rate 100 mL/minute, 60 bar, column temperature 40° C.), eluting with 3% MeOH (+0.1% NH₄OH) in CO₂, to yield, after lyophilisation, the title compounds (Peak 1, 20 mg, 20%; and Peak 2, 23 mg, 23%).

Peak 1: δ_{H} (400 MHz, DMSO-d₆) 9.49 (1H, d, J 9.0 Hz), 8.85 (1H, d, J 9.0 Hz), 8.47 (1H, d, J 2.0 Hz), 8.26 (1H, s), 7.93 (1H, d, J 2.0 Hz), 6.10 (1H, tt, J 56.0, 4.5 Hz), 5.20 (1H, t, J 8.5 Hz), 4.64-4.54 (1H, m), 3.96 (1H, t, J 8.0 Hz), 2.77-2.62 (1H, m), 2.45-2.36 (1H, m), 2.32-2.25 (1H, m), 2.23-2.15 (1H, m), 2.09-2.17 (5H, m), 1.67-1.61 (1H, m), 1.44-1.26 (5H, m), 1.14-1.09 (2H, m), 0.98-0.94 (2H, m). LCMS (Method 8): [M+H]⁺ m/z 620, RT 2.15 minutes. Chiral SFC (Chiralpak IC 150×4.6 mm, 3 μm column, flow rate 3 mL/minute, eluting with a 3-40% MeOH (+0.1% NH₄OH) method, using a 6.5 minute run time) RT 1.67 minutes (>99%).
Peak 2: δ_{H} (400 MHz, DMSO-d₆) 9.50 (1H, d, J 9.0 Hz), 8.91 (1H, d, J 9.0 Hz), 8.52 (1H, d, J 2.0 Hz), 8.25 (1H, s), 7.97 (1H, d, J 2.0 Hz), 6.05 (1H, tt, J 56.0, 4.0 Hz), 5.19 (1H, t, J 9.0 Hz), 4.59-4.49 (1H, m), 3.95 (1H, dd, J 9.0, 6.5 Hz), 2.77-2.62 (1H, m), 2.48-2.33 (1H, m), 2.31-2.25 (1H, m), 2.25-2.21 (1H, m), 2.09-1.71 (5H, m), 1.63-1.60 (1H, m), 1.45-1.24 (2H, m), 1.16-1.10 (5H, m), 0.98-0.94 (2H, m). LCMS (Method 8): [M+H]⁺ m/z 620, RT 2.17 minutes. Chiral SFC (Chiralpak IC 150×4.6 mm, 3 μm column, flow rate 3 mL/minute, eluting with a 3-40% MeOH (+0.1% NH₄OH) method, using a 6.5 minute run time) RT 1.85 minutes (98%).

### Example 11

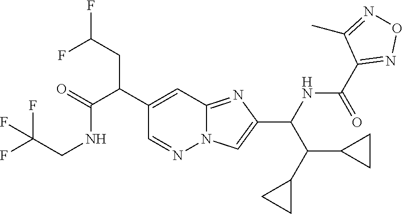 [SMILES: Cc1nonc1C(=O)NC(c1cn2ncc(C(CC(F)F)C(=O)NCC(F)(F)F)cc2n1)C(C1CC1)C1CC1]

### N-(2,2-Dicyclopropyl-1-{7-[3,3-difluoro-1-(2,2,2-trifluoroethylcarbamoyl)propyl]-imidazo[1,2-b]pyridazin-2-yl}ethyl)-4-methyl-1,2,5-oxadiazole-3-carboxamide

Intermediate 42 (140 mg, 0.295 mmol) in DMA (1 mL) was added to a solution of 2-chloro-1-methylpyridinium iodide (98 mg, 0.383 mmol), 2,2,2-trifluoroethanamine (52.6 mg, 0.531 mmol) and DIPEA (154.6 μL, 0.885 mmol) in DMA (1 mL). The reaction mixture was heated at 50° C. in a sealed tube for 2 h, then diluted with water (10 mL) and extracted with ethyl acetate (2×15 mL). The organic extracts were dried with sodium sulfate and concentrated under vacuum. The residue was purified by column chromatography, eluting with a gradient of 0-50% ethyl acetate in heptane, to afford the title compound (131 mg, 80%) as an off-white solid. δ_{H} (500 MHz, CD₃OD) 8.46 (d, J 2.1 Hz, 1H), 8.15 (s, 1H), 7.90 (d, J 1.8 Hz, 1H), 5.95 (tt, J 56.2, 4.3 Hz, 1H), 5.61 (d, J 6.0 Hz, 1H), 3.97 (dd, J 8.9, 5.9 Hz, 1H), 3.95-3.87 (m, 2H), 2.85-2.69 (m, 1H), 2.53 (s, 3H), 2.46-2.25 (m, 1H), 1.74-1.62 (m, 1H), 0.81-0.71 (m, 2H), 0.53-0.44 (m, 2H), 0.40-0.31 (m, 2H), 0.31-0.23 (m, 2H), 0.20-0.11 (m, 1H), 0.10-0.01 (m, 1H). LCMS (Method 2): [M+H]⁺ m/z 556.3, RT 3.69 minutes.

### Example 12

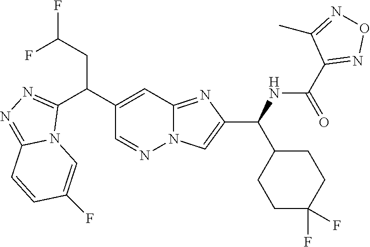 [SMILES: Cc1nonc1C(=O)N[C@H](c1cn2ncc(C(CC(F)F)c3nnc4ccc(F)cn34)cc2n1)C1CCC(F)(F)CC1]

### N-[(S)-(4,4-Difluorocyclohexyl){7-[(1S)-3,3-difluoro-1-(6-fluoro-[1,2,4]triazolo[4,3-a]-pyridin-3-yl)propyl]imidazo[1,2-b]pyridazin-2-yl}methyl]-4-methyl-1,2,5-oxadiazole-3-carboxamide

### N-[(S)-(4,4-Difluorocyclohexyl){7-[(1R)-3,3-difluoro-1-(6-fluoro-[1,2,4]triazolo[4,3-a]-pyridin-3-yl)propyl]imidazo[1,2-b]pyridazin-2-yl}methyl]-4-methyl-1,2,5-oxadiazole-3-carboxamide

To a solution of Intermediate 62 (50 mg, 0.10 mmol), 4-methyl-1,2,5-oxadiazole-3-carboxylic acid (17 mg, 0.13 mmol) and DIPEA (0.05 mL, 0.31 mmol) in DMF (0.52 mL) was added HATU (49 mg, 0.13 mmol). The reaction mixture was stirred at r.t. for 10 minutes, then diluted with DCM (5 mL) and water (5 mL). The aqueous layer was extracted with DCM (2×5 mL). The combined organic extracts were passed through a phase separator and concentrated in vacuo. The crude material was purified by silica column chromatography, eluting with a gradient of 0-100% EtOAc in isohexane, followed by preparative HPLC, to give a mixture of two stereoisomers. The desired stereoisomer was isolated by chiral HPLC (Chiralpak IC 250×20 mm, 5 μm, eluting with 3-40% MeOH (+0.1% NH₄OH) at 100 mL/minute) as the second-eluting peak, RT 7.29 minutes, to give the title compound (3.5 mg, 27%) as a colourless amorphous solid. The undesired first-eluting peak has RT 6.91 minutes. The absolute stereochemistry of the carbon atom adjacent to the difluoroethyl group is unknown. δ_{H} (400 MHz, DMSO-d₆) 9.41 (d, J 9.0 Hz, 1H), 8.90-8.88 (m, 1H), 8.60 (d, J 2.0 Hz, 1H), 8.23 (s, 1H), 8.16 (d, J 2.0 Hz, 1H), 7.92-7.88 (m, 1H), 7.53-7.48 (m, 1H), 6.19 (tt, J 56.0, 4.5 Hz, 1H), 5.15 (t, J 8.5 Hz, 1H), 5.08 (t, J 7.5 Hz, 1H), 3.13-2.88 (m, 2H), 2.46 (s, 3H), 2.19-2.13 (m, 1H), 2.08-1.87 (m, 3H), 1.83-1.69 (m, 2H), 1.59-1.56 (m, 1H), 1.41-1.21 (m, 2H). LCMS (Method 8): [M+H]⁺ m/z 590, RT 1.76 minutes. Chiral LC (Chiralpak IC 150×4.6 mm, 3 μm, eluting with 3-40% MeOH (+0.1% NH₄OH) at 3 mL/minute, 6.5 minutes): RT 4.54 minutes.

### Example 13

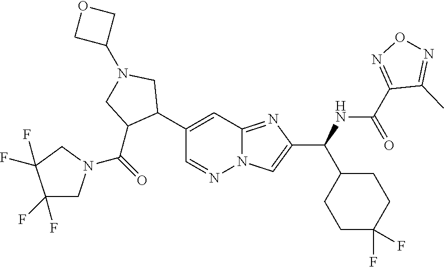 [SMILES: Cc1nonc1C(=O)N[C@H](c1cn2ncc(C3CN(C4COC4)CC3C(=O)N3CC(F)(F)C(F)(F)C3)cc2n1)C1CCC(F)(F)CC1]

### N-[(S)-(4,4-Difluorocyclohexyl){7-[1-(oxetan-3-yl)-4-(3,3,4,4-tetrafluoropyrrolidine-1-carbonyl)pyrrolidin-3-yl]imidazo[1,2-b]pyridazin-2-yl}methyl]-4-methyl-1,2,5-oxadiazole-3-carboxamide

To a solution of Intermediate 54 (63 mg, 0.10 mmol) in DCM (3 mL) were added oxetan-3-one (7.3 μL, 0.11 mmol) and acetic acid (6.48 μL, 0.11 mmol). The mixture was stirred for 5 minutes, then sodium triacetoxyborohydride (35 mg, 0.16 mmol) was added. The mixture was stirred for 18 h, then further portions of oxetan-3-one (7.3 μL, 0.11 mmol) and sodium triacetoxyborohydride (35 mg, 0.16 mmol) were added. The reaction mixture was stirred at r.t for 5 h, then partitioned between DCM (20 mL) and brine (20 mL). The layers were separated, and the aqueous layer was re-extracted with DCM (2×20 mL). The combined organic extracts were passed down a phase separator and concentrated in vacuo. The residue was purified by flash column chromatography, eluting with a gradient of 0-40% MeOH in EtOAc. The resulting material (1:1 mixture of pyrrolidine cis isomers) was subjected to chiral SFC purification (Chiralpak IC 250×20 mm, 5 μm column, flow rate 100 mL/minute, column temperature 40° C., eluting with a 3-40% MeOH (+0.1% NH₄OH) method), to yield the title compound (9 mg, 13%) as the first-eluting peak. The absolute stereochemistry of the pyrrolidine ring is unknown. δ_{H} (400 MHz, DMSO-d₆) 9.43 (d, J 9.0 Hz, 1H), 8.47 (d, J 2.1 Hz, 1H), 8.19 (s, 1H), 8.00 (d, J 2.0 Hz, 1H), 5.17 (t, J 8.5 Hz, 1H), 4.63 (td, J 6.5, 4.1 Hz, 2H), 4.53 (dt, J 13.8, 5.9 Hz, 2H), 4.40 (q, J 13.9 Hz, 1H), 4.25 (q, J 13.4 Hz, 1H), 3.94-3.85 (m, 1H), 3.79 (ddd, J 11.3, 9.4, 5.9 Hz, 2H), 3.68 (td, J 9.1, 6.5 Hz, 1H), 3.33-3.17 (m, 2H), 3.13 (dd, J 9.4, 6.4 Hz, 1H), 2.96 (dd, J 9.1, 6.5 Hz, 1H), 2.87-2.76 (m, 2H), 2.56-2.54 (m, 1H), 2.46 (s, 3H), 2.17 (d, J 10.0 Hz, 1H), 2.10-1.91 (m, 1H), 1.91-1.67 (m, 2H), 1.66-1.51 (m, 1H), 1.45-1.31 (m, 1H), 1.31-1.15 (m, 1H). LCMS (Method 8) [M+H]⁺ m/z 671, RT 1.81 minutes. LCMS (Method 10) [M+H]⁺ m/z 671, RT 1.63 minutes. Chiral SFC (Chiralpak IC 150×4.6 mm, 3 μm column, flow rate 3 mL/minute, eluting with a 3-40% MeOH (+0.1% NH₄OH) method, using a 6.5 minute run time): RT 3.99 minutes.

### Example 14

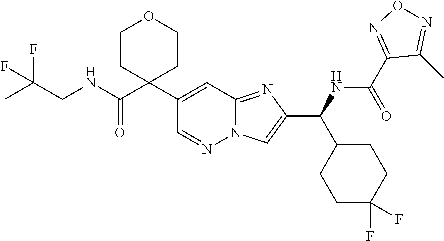 [SMILES: Cc1nonc1C(=O)N[C@H](c1cn2ncc(C3(C(=O)NCC(C)(F)F)CCOCC3)cc2n1)C1CCC(F)(F)CC1]

### N-[(S)-(4,4-Difluorocyclohexyl){7-[4-(2,2-difluoropropylcarbamoyl)tetrahydropyran-4-yl]imidazo[1,2-b]pyridazin-2-yl}methyl]-4-methyl-1,2,5-oxadiazole-3-carboxamide

Intermediate 69 (50 mg, 0.095 mmol), DIPEA (0.03 mL, 0.2 mmol) and 4-methyl-1,2,5-oxadiazole-3-carboxylic acid (15 mg, 0.11 mmol) were dissolved in DCM (5 mL), then HATU (50 mg, 0.13 mmol) was added. The reaction mixture was stirred overnight, then diluted with DCM (10 mL), washed with water (10 mL), passed through a hydrophobic frit and evaporated. The residue was dissolved in MeOH, then purified by preparative HPLC, to afford the title compound (10 mg, 18%) as a white solid. δ_{H} (300 MHz, DMSO-d₆) 9.42 (d, J 8.9 Hz, 1H), 8.43 (d, J 2.2 Hz, 1H), 8.24 (s, 1H), 8.18 (t, J 6.2 Hz, 1H), 7.95 (d, J 2.3 Hz, 1H), 5.16 (d, J 8.5 Hz, 1H), 3.78 (d, J 11.8 Hz, 2H), 3.60-3.42 (m, 4H), 2.47 (s, 3H), 2.33-1.25 (m, 13H), 1.48 (t, J 19.3 Hz, 3H). LCMS (Method 7): [M+H]⁺ m/z 582, RT 2.15 minutes.

### Example 15

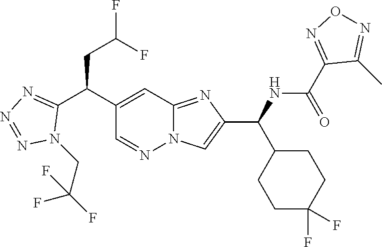 [SMILES: Cc1nonc1C(=O)N[C@H](c1cn2ncc([C@H](CC(F)F)c3nnnn3CC(F)(F)F)cc2n1)C1CCC(F)(F)CC1]

### N-[(S)-(4,4-Difluorocyclohexyl)(7-{(1S*)-3,3-difluoro-1-[1-(2,2,2-trifluoroethyl)tetrazol-5-yl]propyl}imidazo[1,2-b]pyridazin-2-yl)methyl]-4-methyl-1,2,5-oxadiazole-3-carboxamide

HATU (111 mg, 0.291 mmol) was added to a mixture of 4-methyl-1,2,5-oxadiazole-3-carboxylic acid (37 mg, 0.291 mmol) and DIPEA (0.081 mL, 0.466 mmol) in anhydrous DMF (3 mL) at r.t. The reaction mixture was stirred at r.t. for 5 minutes, then Intermediate 47 (96 mg, 0.194 mmol) was added. The reaction mixture was stirred at r.t. for 16 h, then diluted with EtOAc (50 mL) and washed with water (10 mL). The organic phase was dried over sodium sulfate, then filtered and concentrated to dryness in vacuum. The resulting material was purified by FCC (Biotage Isolera, SiO₂, gradient elution with 10-50% EtOAc:heptanes), followed by chiral preparative SFC LCMS (Waters Thar 3100 SFC system connected to a Waters 2998 PDA detector using Chiralcel OJ-H, 10×250 mm, 5 μm, eluting with 8% acetonitrile:92% CO₂ at 15 mL/minute), to give the title compound (desired isomer) (5.7 mg). The stereochemistry adjacent to the tetrazole carbon has been arbitrarily assigned. The second isomer (undesired) was also isolated. δ_{H} (400 MHz, CDCl₃) 8.52 (s, 1H), 8.08 (d, J 13.1 Hz, 1H), 7.94 (s, 1H), 7.85 (d, J 11.6 Hz, 1H), 5.95 (tt, J 55.5, 3.8 Hz, 1H), 5.28 (t, J 8.3 Hz, 1H), 5.00 (p, J 9.2, 8.5 Hz, 2H), 4.78-4.61 (m, 1H), 3.13-2.97 (m, 1H), 2.78-2.63 (m, 1H), 2.60 (s, 3H), 2.28-2.11 (m, 2H), 2.11-1.96 (m, 2H), 1.83-1.66 (m, 3H), 1.56-1.47 (m, 1H), 1.43-1.33 (m, 1H). LCMS (Method 2): [M+H]⁺ m/z 605, RT 3.52 minutes. Chiral LC (Waters Thar 3100 SFC system connected to a Waters 2998 PDA detector using Cellulose-3, 4.6×250 mm, 5 μm, eluting with 15% acetonitrile:85% CO₂ at 4 mL/minute, 6 minutes): RT 1.77 minutes (undesired isomer: RT 3.29 minutes).

### Example 16

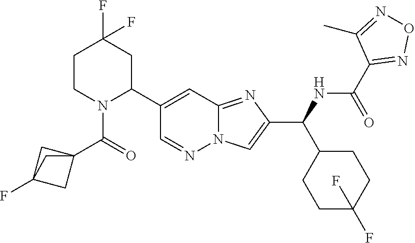

### N-[(S)-(4,4-Difluorocyclohexyl){7-[4,4-difluoro-1-(3-fluorobicyclo[1.1.1]pentane-1-carbonyl)piperidin-2-yl]imidazo[1,2-b]pyridazin-2-yl}methyl]-4-methyl-1,2,5-oxadiazole-3-carboxamide

A solution of Intermediate 57 (46 mg, 76 μmol) and TFA (1.0 mL) in DCM (1.5 mL) was stirred at r.t. for 2 h, then the reaction mixture was concentrated. The residue was dissolved in MeOH (0.5 mL). The solution was loaded onto an SCX column and eluted with MeOH, then 7N methanolic ammonia. The basic eluent was concentrated. The residue was mixed with 3-fluorobicyclo[1.1.1]pentane-1-carboxylic acid (32.0 mg, 0.25 mmol), HATU (94.0 mg, 0.25 mmol), DIPEA (60 μL, 0.34 mmol), and DMF (1.0 mL). The mixture was stirred at r.t. for 1 h, then concentrated under reduced pressure. The residue was purified by flash column chromatography, eluting with a gradient of 0-50% EtOAc in hexanes. Diastereomer separation of the resulting yellow solid (30 mg) was performed on an SFC Prep 100 with Chiralpak IC 150×4.6 mm, 3 μm column (flow rate 3 mL/minute, eluting with 3-40% methanol (+0.1% ammonia solution) over a 7.5 minute run time), to give the title compound (first-eluted peak) (9 mg, 20%), together with the opposite diastereomer. The absolute stereochemistry at the 2-position of the piperidine ring is unknown. δ_{H} (400 MHz, 373K, DMSO-d₆) 8.95 (s, 1H), 8.41 (d, J 2.2 Hz, 1H), 8.20 (s, 1H), 7.79 (d, J 2.1 Hz, 1H), 5.81 (s, 1H), 5.23 (d, J 7.7 Hz, 1H), 4.27 (d, J 14.5 Hz, 1H), 3.25 (s, 1H), 2.22 (d, J 9.9 Hz, 1H), 2.17-1.99 (m, 4H), 1.94 (m, 1H), 1.89-1.69 (m, 3H), 1.57-1.27 (m, 2H). Additional proton signals obscured by solvent peaks. LCMS (Method 8): [M+H]⁺ m/z 608.4, RT 2.26 minutes. Chiral LC (Waters UPC2—Qda system, Chiralpak IC, 150×4.6 mm, 3 μm, eluting with 3-40% methanol (+0.1% ammonia solution) at 3 mL/minute, 6.5 minute run): RT 2.93 minutes.

### Example 17

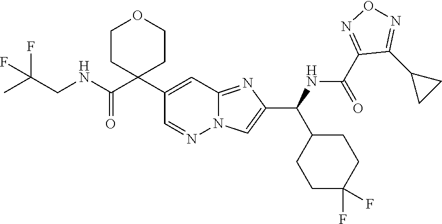 [SMILES: CC(F)(F)CNC(=O)C1(c2cnn3cc([C@@H](NC(=O)c4nonc4C4CC4)C4CCC(F)(F)CC4)nc3c2)CCOCC1]

### 4-Cyclopropyl-N-[(S)-(4,4-difluorocyclohexyl){7-[4-(2,2-difluoropropylcarbamoyl)-tetrahydropyran-4-yl]imidazo[1,2-b]pyridazin-2-yl}methyl]-1,2,5-oxadiazole-3-carboxamide

Intermediate 69 (50 mg, 0.095 mmol), DIPEA (0.03 mL, 0.2 mmol) and 4-cyclopropyl-1,2,5-oxadiazole-3-carboxylic acid (20 mg, 0.13 mmol) were dissolved in DCM (5 mL), then HATU (50 mg, 0.13 mmol) was added. The reaction mixture was stirred overnight, then diluted with DCM (10 mL), washed with water (10 mL), passed through a hydrophobic frit and evaporated in vacuo. The residue was dissolved in MeOH, then purified by basic C18, preparative HPLC, to afford the title compound (26 mg, 44%) as a white solid. δ_{H} (400 MHz, DMSO-d₆) 9.49 (d, J 9.0 Hz, 1H), 8.43 (d, J 2.2 Hz, 1H), 8.24 (s, 1H), 8.18 (t, J 6.3 Hz, 1H), 7.97-7.92 (m, 1H), 5.20 (t, J 8.5 Hz, 1H), 3.78 (d, J 11.9 Hz, 2H), 3.51 (td, J 14.3, 6.1 Hz, 4H), 2.49 (m, 1H), 2.29 (dt, J 8.4, 5.0 Hz, 1H), 2.19 (m, 1H), 2.10-1.70 (m, 8H), 1.63 (m, 1H), 1.47 (dd, J 19.8, 14.1 Hz, 3H), 1.40-1.22 (m, 2H), 1.13 (m, 2H), 0.97 (m, 2H). LCMS (Method 8): [M+H]*m/z 608.2, RT 1.96 minutes.

1. A compound of formula (I) or an N-oxide thereof, or a pharmaceutically acceptable salt thereof: 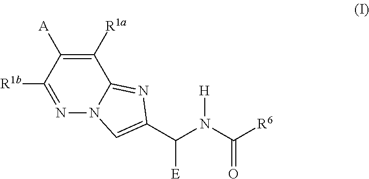
wherein
E represents a group of formula (Ea), (Eb), (Ec), (Ed) or (Ee): 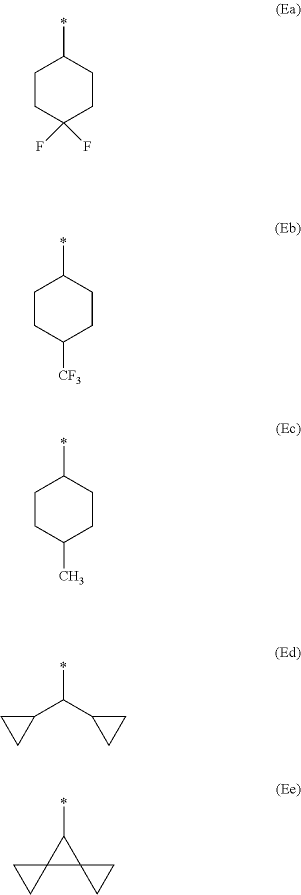
in which the asterisk (*) represents the point of attachment to the remainder of the molecule;
A represents a group of formula (Aa), (Ab), (Ac), (Ad) or (Ae): 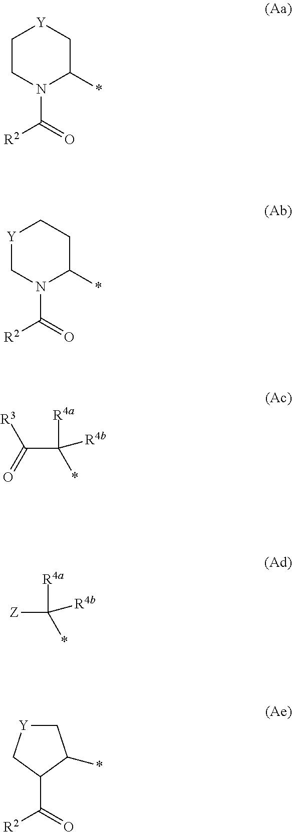
in which the asterisk (*) represents the point of attachment to the remainder of the molecule;
Y represents —O—, —N(R⁷)—, —C(R^{5a})(R^{5b})—, —S—, —S(O)—, —S(O)₂— or —S(O)(N—R⁸)—;
Z represents heteroaryl, which group may be optionally substituted by one or more substituents;
R^{1a} represents hydrogen, fluoro, chloro, methyl, difluoromethyl or trifluoromethyl;
R^{1b} represents hydrogen, fluoro, chloro, methyl, difluoromethyl or trifluoromethyl;
R² represents —OR^{2a}; or R² represents C₃₋₉ cycloalkyl, C₄-1₂ bicycloalkyl, C₃₋₇ heterocycloalkyl or C₄₋₉ heterobicycloalkyl, any of which groups may be optionally substituted by one or more substituents;
R^{2a} represents C₁₋₆ alkyl; or R^{2a} represents C₃₋₉ cycloalkyl, which group may be optionally substituted by one or more substituents;
R³ represents —NR^{3a}R^{3b}; or R³ represents a group of formula (Wa): 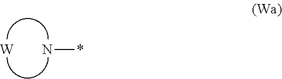
in which the asterisk (*) represents the point of attachment to the remainder of the molecule;
W represents the residue of an optionally substituted saturated monocyclic ring containing 3 to 6 carbon atoms, one nitrogen atom, and 0, 1, 2 or 3 additional heteroatoms independently selected from N, O and S, but containing no more than one O or S atom; or
W represents the residue of an optionally substituted saturated bicyclic ring system containing 4 to 10 carbon atoms, one nitrogen atom, and 0, 1, 2 or 3 additional heteroatoms independently selected from N, O and S, but containing no more than one O or S atom; or
W represents the residue of an optionally substituted saturated spirocyclic ring system containing 5 to 10 carbon atoms, one nitrogen atom, and 0, 1, 2 or 3 additional heteroatoms independently selected from N, O and S, but containing no more than one O or S atom;
R^{3a} represents hydrogen or C₁₋₆ alkyl;
R^{3b} represents C₁₋₆ alkyl, C₃₋₇ cycloalkyl, C₃₋₇ cycloalkyl(C₁₋₆)alkyl, C₄₋₁₂ bicycloalkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents;
R^{4a} represents hydrogen, fluoro or hydroxy; or R^{4a} represents C₁₋₆ alkyl, which group may be optionally substituted by one or more substituents; and
R^{4b} represents hydrogen or fluoro; or R^{4b} represents C₁₋₆ alkyl, which group may be optionally substituted by one or more substituents; or
R^{4a} and R^{4b}, when taken together with the carbon atom to which they are both attached, represent C₃₋₉ cycloalkyl or C₃₋₇ heterocycloalkyl, either of which groups may be optionally substituted by one or more substituents;
R^{5a} represents hydrogen, fluoro, methyl, difluoromethyl or trifluoromethyl; and
R^{5b} represents hydrogen, fluoro, methyl or hydroxy; or
R^{5a} and R^{5b}, when taken together with the carbon atom to which they are both attached, represent cyclopropyl;
R⁶ represents —OR^{6a} or —NR^{6b}R^{6c}; or R⁶ represents C₁₋₆ alkyl, C₃₋₉ cycloalkyl, C₃₋₉ cycloalkyl(C₁₋₆)alkyl, aryl, aryl(C₁₋₆)alkyl, C₃₋₇ heterocycloalkyl, C₃₋₇ heterocycloalkyl-(C₁₋₆)alkyl, heteroaryl or heteroaryl(C₁₋₆)alkyl, any of which groups may be optionally substituted by one or more substituents;
R^{6a} represents C₁₋₆ alkyl; or R^{6a} represents C₃₋₉ cycloalkyl or C₃₋₇ heterocycloalkyl, either of which groups may be optionally substituted by one or more substituents;
R^{6b} represents hydrogen or C₁₋₆ alkyl; and
R^{6c} represents hydrogen or C₁₋₆ alkyl; or
R^{6b} and R^{6c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents;
R⁷ represents —COR^{7a}, —CO₂R^{7a} or —SO₂R^{7b}; or R⁷ represents hydrogen; or R⁷ represents C₁₋₆ alkyl, C₃₋₉ cycloalkyl, or C₃₋₇ heterocycloalkyl, any of which groups may be optionally substituted by one or more fluorine atoms;
R^{7a} represents C₁₋₆ alkyl, optionally substituted by one or more fluorine atoms;
R^{7b} represents C₁₋₆ alkyl; and
R³ represents C₁₋₆ alkyl.

2. A compound as claimed in claim 1 wherein E represents a group of formula (Ea) or (Ed) as defined in claim 1.

3. A compound as claimed in claim 1 wherein A represents a group of formula (Aa), (Ac), (Ad) or (Ae) as defined in claim 1.

4. A compound as claimed in claim 1 wherein R⁶ represents heteroaryl, which group may be optionally substituted by one or more substituents.

5. A compound as claimed in claim 1 represented by formula (IIA-1) or an N-oxide thereof, or a pharmaceutically acceptable salt thereof: 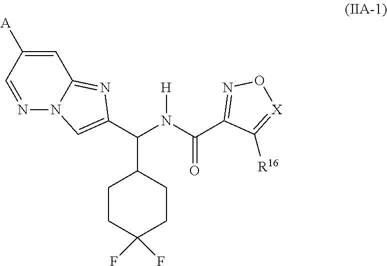
wherein
X represents CH or N;
R¹⁶ represents methyl, ethyl, isopropyl or cyclopropyl; and
A is as defined in claim 1.

6. A compound as claimed in claim 1 represented by formula (IIA-2) or an N-oxide thereof, or a pharmaceutically acceptable salt thereof: 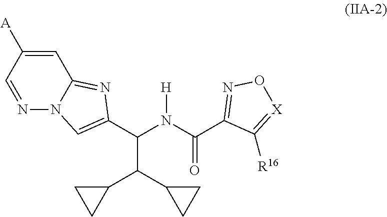
wherein
A is as defined in claim 1;
X represents CH or N; and
R¹⁶ represents methyl, ethyl, isopropyl or cyclopropyl.

7. A compound as claimed in claim 1 represented by formula (IIB-1) or an N-oxide thereof, or a pharmaceutically acceptable salt thereof: 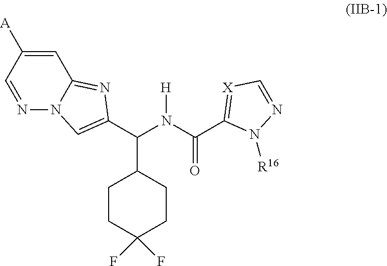
wherein
A is as defined in claim 1;
X represents CH or N; and
R¹⁶ represents methyl, ethyl, isopropyl or cyclopropyl.

8. A compound as claimed in claim 1 represented by formula (IIB-2) or an N-oxide thereof, or a pharmaceutically acceptable salt thereof: 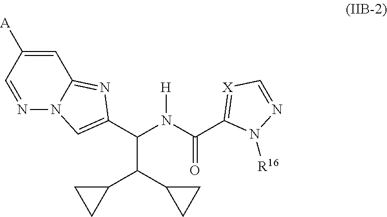
wherein
A is as defined in claim 1;
X represents CH or N; and
R¹⁶ represents methyl, ethyl, isopropyl or cyclopropyl.

9. A compound as claimed in claim 1 which is:
N-[(S)-(4,4-Difluorocyclohexyl){7-[3,3-difluoro-1-(2,2,2-trifluoroethylcarbamoyl)-propyl]imidazo[1,2-b]pyridazin-2-yl}methyl]-4-methyl-1,2,5-oxadiazole-3-carboxamide;
N-{(S)-(4,4-difluorocyclohexyl)[7-(3,3-difluoro-1-{[(1R)-2-methyl-1-(methylcarbamoyl)-propyl]carbamoyl}propyl)imidazo[1,2-b]pyridazin-2-yl]methyl}-4-methyl-1,2,5-oxadiazole-3-carboxamide;
N-[(S)-{7-[(1R)-1-{[(1 S)-1-Cyclopropyl-2,2,2-trifluoroethyl]carbamoyl}-3,3-difluoro-propyl]imidazo[1,2-b]pyridazin-2-yl}(4,4-difluorocyclohexyl)methyl]-4-methyl-1,2,5-oxadiazole-3-carboxamide;
N-[(S)-{7-[(1 S)-1-{[(1 S)-1-Cyclopropyl-2,2,2-trifluoroethyl]carbamoyl}-3,3-difluoro-propyl]imidazo[1,2-b]pyridazin-2-yl}(4,4-difluorocyclohexyl)methyl]-4-methyl-1,2,5-oxadiazole-3-carboxamide;
4-Cyclopropyl-N-[(S)-(4,4-difluorocyclohexyl){7-[(1R)-3,3-difluoro-1-(2,2,2-trifluoro-ethylcarbamoyl)propyl]imidazo[1,2-b]pyridazin-2-yl}methyl]-1,2,5-oxadiazole-3-carboxamide;
4-Cyclopropyl-N-[(S)-(4,4-difluorocyclohexyl){7-[(1R)-3,3-difluoro-1-(2,2,2-trifluoro-ethylcarbamoyl)propyl]imidazo[1,2-b]pyridazin-2-yl}methyl]-1,2,5-oxadiazole-3-carboxamide;
N-[(S)-(4,4-Difluorocyclohexyl){7-[3,3-difluoro-1-(2,2,2-trifluoroethylcarbamoyl)-propyl]imidazo[1,2-b]pyridazin-2-yl}methyl]-4-ethyl-1,2,5-oxadiazole-3-carboxamide;
N-[(S)-(4,4-Difluorocyclohexyl){7-[(1R)-3,3-difluoro-1-(2,2,2-trifluoroethylcarbamoyl)-propyl]imidazo[1,2-b]pyridazin-2-yl}methyl]-4-ethylisoxazole-3-carboxamide;
N-[(S)-(4,4-Difluorocyclohexyl){7-[(1 S)-3,3-difluoro-1-(2,2,2-trifluoroethylcarbamoyl)-propyl]imidazo[1,2-b]pyridazin-2-yl}methyl]-4-ethylisoxazole-3-carboxamide;
N-[(S)-(4,4-Difluorocyclohexyl){7-[3,3-difluoro-1-(2,2,2-trifluoroethylcarbamoyl)-propyl]imidazo[1,2-b]pyridazin-2-yl}methyl]-2-isopropylpyrazole-3-carboxamide;
N-[(S)-(4,4-Difluorocyclohexyl){7-[3,3-difluoro-1-(2,2,2-trifluoroethylcarbamoyl)-propyl]imidazo[1,2-b]pyridazin-2-yl}methyl]-2-isopropyl-1,2,4-triazole-3-carboxamide;
N-[(S)-(4,4-Difluorocyclohexyl){7-[4,4-difluoro-1-(2,2,2-trifluoroethylcarbamoyl)-cyclohexyl]imidazo[1,2-b]pyridazin-2-yl}methyl]-4-methyl-1,2,5-oxadiazole-3-carboxamide;
4-Cyclopropyl-N-[(S)-(4,4-difluorocyclohexyl){7-[(1 S)-3,3-difluoro-1-{[(1 S)-2,2,2-trifluoro-1-methylethyl]carbamoyl}propyl]imidazo[1,2-b]pyridazin-2-yl}methyl]-1,2,5-oxadiazole-3-carboxamide;
4-Cyclopropyl-N-[(S)-(4,4-difluorocyclohexyl){7-[(1R)-3,3-difluoro-1-{[(1S)-2,2,2-trifluoro-1-methylethyl]carbamoyl}propyl]imidazo[1,2-b]pyridazin-2-yl}methyl]-1,2,5-oxadiazole-3-carboxamide;
N-(2,2-Dicyclopropyl-1-{7-[3,3-difluoro-1-(2,2,2-trifluoroethylcarbamoyl)propyl]-imidazo[1,2-b]pyridazin-2-yl}ethyl)-4-methyl-1,2,5-oxadiazole-3-carboxamide;
N-[(S)-(4,4-Difluorocyclohexyl){7-[(1 S)-3,3-difluoro-1-(6-fluoro-[1,2,4]triazolo[4,3-a]-pyridin-3-yl)propyl]imidazo[1,2-b]pyridazin-2-yl}methyl]-4-methyl-1,2,5-oxadiazole-3-carboxamide;
N-[(S)-(4,4-Difluorocyclohexyl){7-[(1R)-3,3-difluoro-1-(6-fluoro-[1,2,4]triazolo[4,3-a]-pyridin-3-yl)propyl]imidazo[1,2-b]pyridazin-2-yl}methyl]-4-methyl-1,2,5-oxadiazole-3-carboxamide;
N-[(S)-(4,4-Difluorocyclohexyl){7-[1-(oxetan-3-yl)-4-(3,3,4,4-tetrafluoropyrrolidine-1-carbonyl)pyrrolidin-3-yl]imidazo[1,2-b]pyridazin-2-yl}methyl]-4-methyl-1,2,5-oxadiazole-3-carboxamide;
N-[(S)-(4,4-Difluorocyclohexyl){7-[4-(2,2-difluoropropylcarbamoyl)tetrahydropyran-4-yl]imidazo[1,2-b]pyridazin-2-yl}methyl]-4-methyl-1,2,5-oxadiazole-3-carboxamide;
N-[(S)-(4,4-Difluorocyclohexyl)(7-{(1 S*)-3,3-difluoro-1-[1-(2,2,2-trifluoroethyl)tetrazol-5-yl]propyl}imidazo[1,2-b]pyridazin-2-yl)methyl]-4-methyl-1,2,5-oxadiazole-3-carboxamide;
N-[(S)-(4,4-Difluorocyclohexyl){7-[4,4-difluoro-1-(3-fluorobicyclo[1.1.1]pentane-1-carbonyl)piperidin-2-yl]imidazo[1,2-b]pyridazin-2-yl}methyl]-4-methyl-1,2,5-oxadiazole-3-carboxamide; or
4-Cyclopropyl-N-[(S)-(4,4-difluorocyclohexyl){7-[4-(2,2-difluoropropylcarbamoyl)-tetrahydropyran-4-yl]imidazo[1,2-b]pyridazin-2-yl}methyl]-1,2,5-oxadiazole-3-carboxamide;
or a pharmaceutically acceptable salt thereof.

10. (canceled)

11. (canceled)

12. (canceled)

13. A pharmaceutical composition comprising a compound of formula (I) as defined in claim 1 or an N-oxide thereof, or a pharmaceutically acceptable salt thereof, in association with a pharmaceutically acceptable carrier.

14. A pharmaceutical composition as claimed in claim 13 further comprising an additional pharmaceutically active ingredient.

15. (canceled)

16. (canceled)

17. A method for the treatment and/or prevention of disorders for which the administration of a modulator of IL-17 function is indicated which comprises administering to a patient in need of such treatment an effective amount of a compound of formula (I) as defined in claim 1 or an N-oxide thereof, or a pharmaceutically acceptable salt thereof.

18. A method for the treatment and/or prevention of an inflammatory or autoimmune disorder, which comprises administering to a patient in need of such treatment an effective amount of a compound of formula (I) as defined in claim 1 or an N-oxide thereof, or a pharmaceutically acceptable salt thereof.

19. A compound as claimed in claim 2 wherein A represents a group of formula (Aa), (Ac), (Ad) or (Ae).

20. A compound as claimed in claim 2 wherein R⁶ represents heteroaryl, which group may be optionally substituted by one or more substituents.

21. A compound as claimed in claim 3 wherein R⁶ represents heteroaryl, which group may be optionally substituted by one or more substituents.
